(19) United States
(12) Patent Application Publication
(10) Pub. No.: US 2024/0294561 A1
(43) Pub. Date: Sep. 5, 2024

(54) Prodrugs of Thyroid Hormone Analogs, Methods of Making and Methods of Using Thereof

(71) Applicant: Gannex, LLC, Raleigh, NC (US)

(72) Inventors: Jinzi Jason Wu, Raleigh, NC (US); Bin Liang, Hangzhou (CN)

(21) Appl. No.: 18/434,654

(22) Filed: Feb. 6, 2024

Related U.S. Application Data
(60) Provisional application No. 63/514,925, filed on Jul. 21, 2023.
(60) Provisional application No. 63/510,810, filed on Jun. 28, 2023.
(60) Provisional application No. 63/504,832, filed on May 30, 2023.
(60) Provisional application No. 63/492,398, filed on Mar. 27, 2023.
(60) Provisional application No. 63/487,794, filed on Mar. 1, 2023.

(30) Foreign Application Priority Data
Feb. 7, 2023 (CN) 202310113004.X
Mar. 15, 2023 (CN) 202310267006.4
May 18, 2023 (CN) 202310571548.0
Jun. 20, 2023 (CN) 202310733484.X

(51) Int. Cl. C07F 9/54; A61K 31/662; A61K 31/665; C07F 9/6574

(52) U.S. Cl. CPC C07F 9/5463; A61K 31/662; A61K 31/665; C07F 9/65742

(57) ABSTRACT

The present application describes a thyroid hormone receptor β subtype agonist derivative, a preparation method and a use thereof.

### CROSS-REFERENCE OF RELATED APPLICATIONS

This application claims the benefit of priority to Chinese Patent Application No. CN202310113004.X, filed Feb. 7, 2023, Chinese Patent Application No. CN202310267006.4, filed Mar. 15, 2023, Chinese Patent Application No. CN202310571548.0, filed May 18, 2023 and Chinese Patent Application No. CN202310733484.X, filed Jun. 20, 2023 and U.S. Provisional Application No. 63/487,794, filed Mar. 1, 2023, U.S. Provisional Application No. 63/492,398, filed Mar. 27, 2023, U.S. Provisional Application No. 63/504,832, filed May 30, 2023, U.S. Provisional Application No. 63/510,810, filed Jun. 28, 2023, and U.S. Provisional Application No. 63/514,925, filed Jul. 21, 2023, which are all incorporated by reference in their entireties herein.

### FIELD

The present invention relates generally to prodrugs to thyroid hormone analogs, and preparation methods thereof, as well as its use.

### BACKGROUND

Thyroid hormones are critical for normal growth and development and for maintaining metabolic homeostasis (Paul M. Yen Physiological Review, Vol. 81(3): pp. 1097-1126 (2001)). Circulating levels of thyroid hormones are tightly regulated by feedback mechanisms in the hypothalamus/pituitary/thyroid (HPT) axis. Thyroid dysfunction leading to hypothyroidism or hyperthyroidism clearly demonstrates that thyroid hormones exert profound effects on cardiac function, body weight, metabolism, metabolic rate, body temperature, cholesterol, bone, muscle and behavior. The thyroid hormone receptors are derived from two separate genes, α and β. These distinct gene products produce multiple forms of their respective receptors through differential RNA processing. The major thyroid receptor isoforms are α1, α2, β1 and β2. Thyroid hormone receptors al, β1 and β2 bind thyroid hormone. It has been shown that the thyroid hormone receptor subtypes can differ in their contribution to particular biological responses. Recent studies suggest that TRβ1 plays an important role in regulating TRH (thyrotropin releasing hormone) and on regulating thyroid hormone actions in the liver. TRβ2 plays an important role in the regulation of TSH (thyroid stimulating hormone). TRβ1 plays an important role in regulating heart rate.

### SUMMARY

One aspect of the present application relates to a compound of Formula I:

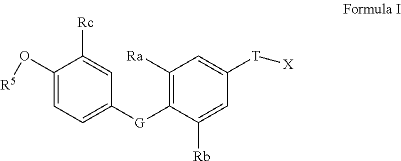

stereoisomers thereof, pharmaceutically acceptable salts thereof, deuterium substitutes thereof, isomers thereof, prodrugs thereof, and metabolites thereof,
   wherein:
G is selected from the group consisting of —O—, and —C(X₉X₈)—;
T is selected from the group consisting of —(CR_{d})ₘ—, and —O—(CR_{d})ₘ—;
m is an integer from 0-3;
X is selected from the group consisting of:

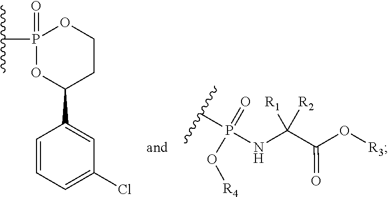

each of Ra and Rb is independently selected from the group consisting of CH₃, CD₃, Cl, Br, I and CF₃;
wherein Re is selected from the group consisting of hydrogen, halogen, —CF₃, —OCF₃, cyano, optionally substituted —C₁-C₁₂ alkyl, optionally substituted —C₂-C₁₂ alkenyl, optionally substituted-C₂-C₁₂ alkynyl, optionally substituted —C₀₋₆ alkyl-aryl, optionally substituted —C₀₋₆ alkyl-cycloalkyl, optionally substituted —C₀₋₆ alkyl-heterocycloalkyl, and optionally substituted —C₃₋₈ cycloalkyl;
wherein R_{c} is optionally substituted with one to ten halogen, H or D;
each of R_{d} is independently selected from the group consisting of hydrogen, halogen, and C₁₋₆ alkyl;
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —COOR₆, CH₂OC(O)OR₇, —CONHR₆, —CONR₇R₁₀, —CONR₆R₇, —CH₂OCOR₆, —CH₂OCONHR₆, and

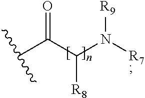

wherein each of R₆ is independently C₁₋₃₀ alkyl, C₁₋₃₀ alkenyl, and C₁₋₃₀ alkynyl, wherein C₁₋₃₀ alkyl, C₁₋₃₀ alkenyl, and C₁₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₇ is selected from the group consisting of H, —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRcC₂₋₃₀ alkenyl, and —C(O) NRCC₂₋₃₀ alkynyl, wherein each of the —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRC₁₋₃₀ alkyl, —C(O) NRCC₂₋₃₀ alkenyl, and —C(O) NRCC₂₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein each of R₈ and R₉ is independently selected from the group consisting of H, OH, C₁₋₆ alkyl, halo, —O—C₁₋₃ alkyl, —S—C₁₋₃ alkyl, —C₃₋₁₀ cycloalkyl, 3-10 membered heterocyclyl, aryl, and 5-10 membered heteroaryl, wherein each of the C₁₋₆ alkyl, —O—C₁₋₃ alkyl, —S—C₁₋₃ alkyl, —C₃₋₁₀ cycloalkyl, 3-10 membered heterocyclyl, aryl, and 5-10 membered heteroaryl can be optionally substituted with one or more substituents each independently selected from the group consisting of OH, halo, —O—C₁₋₃ alkyl, —S—C₁₋₃ alkyl, —C₃₋₁₀ cycloalkyl, 3-10 membered heterocyclyl, aryl, and 5-10 membered heteroaryl;
or wherein R₈ and R₉ are combined with the atoms to which they are attached to form a 3-10 membered heterocyclyl, which is optionally substituted with one to four R₆, wherein each R₆ is independently H, halo, C₁₋₆ alkyl, C₁₋₆ haloalkyl, or alkoxy;
wherein R₁₀ is selected from the group consisting of H, C₁₋₆ alkyl, C₂₋₆ alkenyl, C₂₋₆ alkynyl, —(CH₂)ₙ C₃₋₁₀cycloalkyl, —(CH₂)ₙ aryl, and —(CH₂)ₙ heteroaryl, wherein each of the C₁₋₆ alkyl, C₂₋₆ alkenyl, C₂₋₆ alkynyl, —(CH₂)ₙ C₃₋₁₀cycloalkyl, —(CH₂), aryl, and —(CH₂)ₙ heteroaryl is optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein X₈ and X₉ is independently H, D or halo;
wherein n=0, 1, 2, or 3;
wherein each 5-10 membered heteroaryl mentioned above has one to four heteroatoms, and each is independently N, O, or S; and
wherein each 3-10 membered heterocyclyl mentioned above has one to four heteroatoms, and each is independently N, O, or S,
provided that the compound of formula I is not isopropyl (((4-(4-hydroxy-3-isopropylbenzyl)-3,5-dimethylphenoxy)methyl)(phenoxy)phosphoryl)-L-alaninate.

Another aspect of the present application also relates to a pharmaceutical composition comprising the compound of formula (I) or a stereoisomer thereof, a pharmaceutically acceptable salt thereof, a deuterated compound thereof, a hydrate thereof or a solvate thereof.

Another aspect of the present application relates to a method for treating or preventing obesity, hyperlipidemia, hypercholesterolemia, diabetes mellitus, nonalcoholic steatohepatitis (NASH), hepatic steatosis, arteriosclerosis, cardiovascular disease, hypothyroidism, or thyroid cancer in a subject. The method comprises the step of administering to the subject an effective amount of the compound or the pharmaceutical composition of the present application.

### BRIEF DESCRIPTION OF THE DRAWINGS

FIG. 1. shows the Mean Plasma Concentration-Time Profile of the compounds of present application;

FIG. 2. shows the Mean Plasma Concentration-Time Profile of the compounds of present application;

FIG. 3. shows the Mean Plasma Concentration-Time Profile of Compound 31 after subcutaneous injection;

FIG. 4. shows the Mean Plasma Concentration-Time Profile of Compound 32 after subcutaneous injection;

FIG. 5. shows the Mean Plasma Concentration-Time Profile of Compound 34 after subcutaneous injection;

FIG. 6. shows the Mean Plasma Concentration-Time Profile of Compound 36 after subcutaneous injection;

FIG. 7. shows the Plasma concentration-time profiles of Compound 54 after subcutaneous injection;

FIG. 8. shows the Plasma concentration-time profiles of Compound 52 after subcutaneous injection;

FIG. 9. shows the Plasma concentration-time profiles of Compound 53 after subcutaneous injection;

FIG. 10. shows the Plasma concentration-time profiles of Compound 29 and 30 after subcutaneous injection; and

FIG. 11. shows the Plasma concentration-time profiles of Compound 52 after subcutaneous injection.

### DETAILED DESCRIPTION

### I. Definitions

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cᵤ₋ᵥ” or “Cᵤ-Cᵥ” indicates that the following group has from u to v carbon atoms, where u and v are integers. For example, “C₁₋₆ alkyl” or “C₁-C₆ alkyl” indicates that the alkyl group has from 1 to 6 carbon atoms.

“Alkyl” is a monovalent or divalent linear or branched saturated hydrocarbon radical. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-Propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (1-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃). Alkyl groups can be unsubstituted or substituted.

“Alkenyl” is a monovalent or divalent linear or branched hydrocarbon radical with at least one carbon-carbon double bond. For example, an alkenyl group can have 2 to 8 carbon atoms (i.e., C₂₋₈ alkenyl) or 2 to 6 carbon atoms (i.e., C₂₋₆ alkenyl) or 2 to 4 carbon atoms (i.e., C₂₄ alkenyl). Examples of alkenyl groups include, but are not limited to, ethenyl (—CH═CH₂), allyl (—CH₂CH═CH₂), and —CH₂—CH═CH—CH₃. Alkenyl groups can be unsubstituted or substituted.

“Alkynyl” is a monovalent or divalent linear or branched hydrocarbon radical with at least one carbon-carbon triple bond. For example, an alkynyl group can have 2 to 8 carbon atoms (i.e., C₂₋₈ alkynyl) or 2 to 6 carbon atoms (i.e., C₂₋₆ alkynyl) or 2 to 4 carbon atoms (i.e., C₂₄ alkynyl). Examples of alkynyl groups include, but are not limited to, acetylenyl (—C≡CH), propargyl (—CH₂C≡CH), and —CH₂—C≡C—CH₃. Alkynyl groups can be unsubstituted or substituted.

“Alkoxy” refers to the group —O-alkyl, where alkyl is as defined above. For example, C₁₋₄ alkoxy refers to an —O-alkyl group having 1 to 4 carbons. Alkoxy groups can be unsubstituted or substituted.

“Halogen” or “Halo” refers to fluoro (—F), chloro (—Cl), bromo (—Br) and iodo (—I).

“Haloalkyl” is an alkyl as defined herein, wherein one or more hydrogen atoms of the alkyl are independently replaced by a halogen, which may be the same or different, such that the alkyl is divalent. The alkyl group and the halogen can be any of those described above. In some embodiments, the haloalkyl defines the number of carbon atoms in the alkyl portion, e.g., C₁₋₄ haloalkyl includes CF₃, CH₂F, CHF₂, CH₂CF₃, CH₂CH₂CF₃, CCl₂CH₂CH₂CH₃, and C(CH₃)₂(CF₂H). Haloalkyl groups can be unsubstituted or substituted.

“Aryl” as used herein refers to a monovalent or divalent single all carbon aromatic ring or a multiple condensed all carbon ring system wherein the ring is aromatic. For example, in some embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which multiple rings are aromatic. The rings of the multiple condensed ring system can be connected to each other via fused bonds when allowed by valency requirements. It is also understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl. Non-limiting examples of aryl groups include, but are not limited to, phenyl, naphthyl, anthracenyl, and the like. Aryl groups can be unsubstituted or substituted.

“5-10 membered heteroaryl” or “heteroaryl” refers to a single aromatic ring that has at least one atom other than carbon in the ring, wherein the atom is selected from the group consisting of oxygen, nitrogen and sulfur; “5-10 membered heteroaryl” also includes multiple condensed ring systems that have at least one such aromatic ring, which multiple condensed ring systems are further described below. Thus, “5-10 membered heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary 5-10 membered heteroaryl ring systems include but are not limited to pyridyl, pyrimidinyl, oxazolyl or furyl. “5-10 membered heteroaryl” also includes multiple condensed ring systems (e.g., ring systems comprising 2, 3 or 4 rings) wherein a 5-10 membered heteroaryl group, as defined above, is condensed with one or more rings selected from 5-10 membered heteroaryls (to form for example 1,8-naphthyridinyl) and aryls (to form, for example, benzimidazolyl or indazolyl) to form the multiple condensed ring system. Thus, a 5-10 membered heteroaryl (a single aromatic ring or multiple condensed ring system) can have about 1-20 carbon atoms and about 1-6 heteroatoms within the 5-10 membered heteroaryl ring. For example, tetrazolyl has 1 carbon atom and 4 nitrogen heteroatoms within the ring. The rings of the multiple condensed ring system can be connected to each other via fused bonds when allowed by valency requirements. It is to be understood that the individual rings of the multiple condensed ring system may be connected in any order relative to one another. It is to be understood that the point of attachment for a 5-10 membered heteroaryl or 5-10 membered heteroaryl multiple condensed ring system can be at any suitable atom of the 5-10 membered heteroaryl or 5-10 membered heteroaryl multiple condensed ring system including a carbon atom and a heteroatom (e.g., a nitrogen). It also to be understood that when a reference is made to a certain atom-range membered (e.g., a 5-10 membered heteroaryl), the atom range is for the total ring atoms of the 5-10 membered heteroaryl and includes carbon atoms and heteroatoms. It is also to be understood that the rings of the multiple condensed ring system may include an aryl ring fused to a heterocyclic ring with saturated or partially unsaturated bonds (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. For example, a 5-10 membered heteroaryl includes thiazolyl and a 5-10 membered heteroaryl includes quinolinyl. Exemplary 5-10 membered heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, benzofuranyl, benzimidazolyl, thianaphthenyl, pyrrolo[2,3-b]pyridinyl, quinazolinyl-4(3H)-one, triazolyl, and tetrazolyl. 5-10 membered heteroaryl groups can be unsubstituted or substituted.

“Cycloalkyl” is a monovalent or divalent single all carbon ring or a multiple condensed all carbon ring system wherein the ring in each instance is a non-aromatic saturated or unsaturated ring. For example, in some embodiments, a cycloalkyl group has 3 to 12 carbon atoms, 3 to 10 carbon atoms, 3 to 8 carbon atoms, 3 to 6 carbon atoms, 3 to 5 carbon atoms, or 3 to 4 carbon atoms. Exemplary single ring cycloalkyl groups include cyclopropyl, cyclobutyl, cyclopentyl, cyclohexyl, cyclohexenyl, cycloheptyl, cycloheptenyl, and cyclooctyl. Cycloalkyl also includes multiple condensed ring systems (e.g., ring systems comprising 2 rings) having about 7 to 12 carbon atoms. The rings of the multiple condensed ring system can be connected to each other via fused, spiro, or bridged bonds when allowed by valency requirements. Exemplary multiple ring cycloalkyl groups include octahydropentalene, bicyclo[2.2.1]heptane, bicyclo[2.2.2]octane, bicyclo[2.2.2]oct-2-ene, and spiro[2.5]octane. Cycloalkyl groups can be unsubstituted or substituted.

“Heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular (i.e., ring-shaped) heteroatom selected from oxygen, nitrogen, and sulfur). Unless otherwise specified, a heterocyclyl group has from 3 to about 20 annular atoms, for example from 3 to 12 annular atoms, for example from 4 to 12 annular atoms, 4 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like. Heterocyclyl groups can be unsubstituted or substituted.

“Substituted” as used herein refers to wherein one or more hydrogen atoms of the group are independently replaced by one or more substituents (e.g., 1, 2, 3, or 4 or more) as indicated.

A “compound of the present application” includes compounds disclosed herein, for example a compound of the present application includes compounds of Formula I, including the compounds of the Examples. In some embodiments, a “compound of the present application” includes compounds of Formula I.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Therapeutically effective amount” or “effective amount” as used herein refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present application is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present application within seconds or minutes. In some embodiments, a unit dose of a compound of the present application is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present application. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the subject.

Provided are also pharmaceutically acceptable salts, hydrates, solvates, tautomeric forms, polymorphs, and prodrugs of the compounds described herein.

Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21st Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and N(C₁-C₄ alkyl)₄⁺. Also included are base addition salts, such as sodium or potassium salts.

Provided also are compounds described herein or pharmaceutically acceptable salts, isomers, or a mixture thereof, in which from 1 to n hydrogen atoms attached to a carbon atom may be replaced by a deuterium atom (also referred to as ²H or D), in which n is the number of hydrogen atoms in the molecule. As known in the art, the deuterium atom is a non-radioactive isotope of the hydrogen atom. Such compounds (also referred to as “deuterium substitutes” or “deuterated compounds”) may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Provided also are compounds described herein or pharmaceutically acceptable salts, isomers, or a mixture thereof, in which from 1 to n atoms may be replaced independently by 1 to n corresponding isotopes. Examples of isotopes that can be incorporated into the disclosed compounds also include isotopes of hydrogen, carbon, nitrogen, oxygen, phosphorous, fluorine, chlorine, and iodine, such as—³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, tautomer, diastereomers, and other stereoisomeric forms that may be defined, in terms of absolute stereochemistry, as (R)- or (S)- or, as (D)- or (L)- for amino acids, as well as deuterated analogs thereof. The chemical formula shown in the present application is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Stereoisomer” as used herein refers to a compound made up of the same atoms bonded by the same bonds but having different three-dimensional structures, which are not interchangeable. The present application contemplates various stereoisomers and mixtures thereof and includes “enantiomers”, which refers to two stereoisomers whose molecules are non-superimposable mirror images of one another.

“Tautomer” as used herein refers to a proton shift from one atom of a molecule to another atom of the same molecule. In some embodiments, the present application includes tautomers of said compounds.

“Solvate” as used herein refers to the result of the interaction of a solvent and a compound. Solvates of salts of the compounds described herein are also provided. Hydrates of the compounds described herein are also provided.

“Hydrate” as used herein refers to a compound of the disclosure that is chemically associated with one or more molecules of water.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Prodrug” as used herein refers to a derivative of a drug that upon administration to the human body is converted to the parent drug according to some chemical or enzymatic pathway. In some embodiments, a prodrug is a biologically inactive derivative of a drug that upon administration to the human body is converted to the biologically active parent drug according to some chemical or enzymatic pathway.

“Treatment” or “treat” or “treating” as used herein refers to an approach for obtaining beneficial or desired results. For purposes of the present application, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival. “At risk individual” as used herein refers to an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

### II. Compounds and Compositions

One aspect of the present application relates to a compound of Formula I:

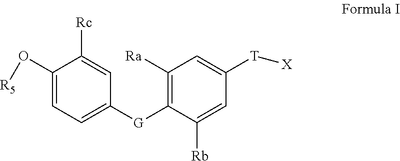

stereoisomers thereof, pharmaceutically acceptable salts thereof, deuterium substitutes thereof, isomers thereof, prodrugs thereof, and metabolites thereof,
   wherein:
G is selected from the group consisting of —O—, and —C(X₉X₈)—;
T is selected from the group consisting of —(CR_{d})ₘ—, and —O—(CRₐ)ₘ—;
m is an integer from 0-3;
X is selected from the group consisting of:

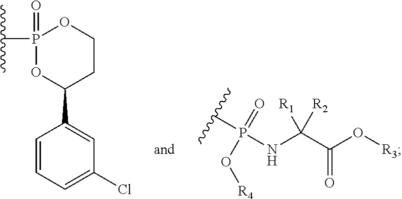

each of Ra and Rb is independently selected from the group consisting of CH₃, CD₃, Cl, Br, I and CF₃;
wherein R_{c} is selected from the group consisting of hydrogen, halogen, —CF₃, —OCF₃, cyano, optionally substituted —C₁-C₁₂ alkyl, optionally substituted —C₂-C₁₂ alkenyl, optionally substituted-C₂-C₁₂ alkynyl, optionally substituted —C₀₋₆alkyl-aryl, optionally substituted —C₀₋₆ alkyl-cycloalkyl, optionally substituted —C₀₋₆ alkyl-heterocycloalkyl, and optionally substituted —C₃₋₈ cycloalkyl;
wherein R_{c} is optionally substituted with one to ten halogen, H or D;
each of R_{d} is independently selected from the group consisting of hydrogen, halogen, and C₁₋₆ alkyl;
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —COOR₆, CH₂OC(O)OR₇, —CONHR₆, —CONR₇R₁₀, —CONR₆R₇, —CH₂OCOR₆, —CH₂OCONHR₆, and

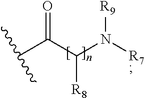

wherein each of R₆ is independently C₁₋₃₀ alkyl, C₁₋₃₀ alkenyl, and C₁₋₃₀ alkynyl, wherein C₁₋₃₀ alkyl, C₁₋₃₀ alkenyl, and C₁₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₇ is selected from the group consisting of H, —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl, wherein each of the —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein each of R₈ and R₉ is independently selected from the group consisting of H, OH, C₁₋₆ alkyl, halo, —O—C₁₋₃ alkyl, —S—C₁₋₃ alkyl, —C₃₋₁₀ cycloalkyl, 3-10 membered heterocyclyl, aryl, and 5-10 membered heteroaryl, wherein each of the C₁₋₆ alkyl, —O—C₁₋₃ alkyl, —S—C₁₋₃ alkyl, —C₃₋₁₀ cycloalkyl, 3-10 membered heterocyclyl, aryl, and 5-10 membered heteroaryl can be optionally substituted with one or more substituents each independently selected from the group consisting of OH, halo, —O—C₁₋₃ alkyl, —S—C₁₋₃ alkyl, —C₃₋₁₀ cycloalkyl, 3-10 membered heterocyclyl, aryl, and 5-10 membered heteroaryl;
or wherein R₈ and R₉ are combined with the atoms to which they are attached to form a 3-10 membered heterocyclyl, which is optionally substituted with one to four R₆, wherein each R₆ is independently H, halo, C₁₋₆ alkyl, C₁₋₆ haloalkyl, or alkoxy;
wherein R₁₀ is selected from the group consisting of H, C₁₋₆ alkyl, C₂₋₆ alkenyl, C₂₋₆ alkynyl, —(CH₂)ₙ C₃₋₁₀cycloalkyl, —(CH₂)ₙ aryl, and —(CH₂)ₙ heteroaryl, wherein each of the C₁₋₆ alkyl, C₂₋₆ alkenyl, C₂₋₆ alkynyl, —(CH₂), C₃₋₁₀cycloalkyl, —(CH₂)ₙ aryl, and —(CH₂)ₙ heteroaryl is optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein X₈ and X₉ is independently H, D or halo;
wherein n=0, 1, 2, or 3;
wherein each 5-10 membered heteroaryl mentioned above has one to four heteroatoms, and each is independently N, O, or S; and
wherein each 3-10 membered heterocyclyl mentioned above has one to four heteroatoms, and each is independently N, O, or S,
provided that the compound of formula I is not isopropyl (((4-(4-hydroxy-3-isopropylbenzyl)-3,5-dimethylphenoxy)methyl)(phenoxy)phosphoryl)-L-alaninate.

In some embodiments,
G is —O—;
T is —O—(CR_{d})ₘ—;
m is an integer from 0-3;
X is

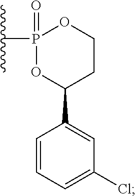 [SMILES: *P1(=O)OCC[C@@H](c2cccc(Cl)c2)O1]

R_{c} is selected from the group consisting of hydrogen, halogen, —CF₃, —OCF₃, cyano, and optionally substituted —C₁-C₁₂ alkyl; and each of Ra is independently selected from the group consisting of hydrogen, halogen, and C₁₋₆ alkyl;

In some embodiments, Rd is hydrogen;

In some embodiments, wherein m is 1.

In some embodiments, the formula I is formula II,

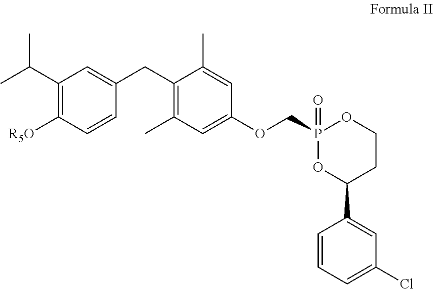

wherein R₅ is selected from the group consisting of —COR₆, —COOR₆, CONR₇R₁₀, —CH₂OCOR₆, CH₂OC(O)OR₇,

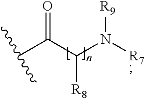

wherein each of R₆ is independently C₁₃₋₃₀ alkyl, C₁₃₋₃₀ alkenyl, and C₁₃₋₃₀ alkynyl, wherein each of the C₁₃₋₃₀ alkyl, C₁₃₋₃₀ alkenyl, and C₁₃₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₇ is selected from the group consisting of H, —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl, wherein each of the —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRC₁₋₃₀ alkyl, —C(O) NRCC₂₋₃₀ alkenyl, and —C(O) NRCC₂₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein each of R₈ and R₉ is independently selected from the group consisting of H, OH, C₁₋₆ alkyl, halo, —O—C₁₋₃ alkyl, —S—C₁₋₃ alkyl, —C₃₋₁₀ cycloalkyl, 3-10 membered heterocyclyl, aryl, and 5-10 membered heteroaryl, wherein each of the C₁₋₆ alkyl, —O—C₁₋₃ alkyl, —S—C₁₋₃ alkyl, —S—S—C(O)OC₂₋₃₀ alkyl, —C₃₋₁₀ cycloalkyl, 3-10 membered heterocyclyl, aryl, and 5-10 membered heteroaryl can be optionally substituted with one or more substituents each independently selected from the group consisting of OH, halo, —O—C₁₋₃ alkyl, —S—C₁₋₃ alkyl, —C₃₋₁₀ cycloalkyl, 3-10 membered heterocyclyl, aryl, and 5-10 membered heteroaryl;
or wherein R₈ and R₉ are combined with the atoms to which they are attached to form a 3-10 membered heterocyclyl, which is optionally substituted with one to four R₆, wherein each R₆ is independently H, halo, C₁₋₆ alkyl, C₁₋₆ haloalkyl, or alkoxy;
wherein R₁₀ is selected from the group consisting of H, C₁₋₆ alkyl, C₂₋₆ alkenyl, C₂₋₆ alkynyl, —(CH₂)ₙ C₃₋₁₀cycloalkyl, —(CH₂)ₙ aryl, and —(CH₂)ₙ heteroaryl, wherein each of the C₁₋₆ alkyl, C₂₋₆ alkenyl, C₂₋₆ alkynyl, —(CH₂)ₙ C₃₋₁₀cycloalkyl, —(CH₂), aryl, and —(CH₂)ₙ heteroaryl is optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein n=0, 1, 2, or 3;
wherein each 5-10 membered heteroaryl mentioned above has one to four heteroatoms, each is independently N, O, or S; and
   wherein each 3-10 membered heterocyclyl mentioned above has one to four heteroatoms, each is independently N, O, or S.

In some embodiments, wherein R₅ is selected from the group consisting of —COR₆, —COOR₆, CONR₇R₁₀, —CH₂OCOR₆, and

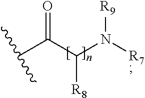

In some embodiments, wherein each of R₆ is independently C₁₃₋₃₀ alkyl and C₁₃₋₃₀ alkenyl;

In some embodiments, the compound of the present application is selected from the group consisting of:

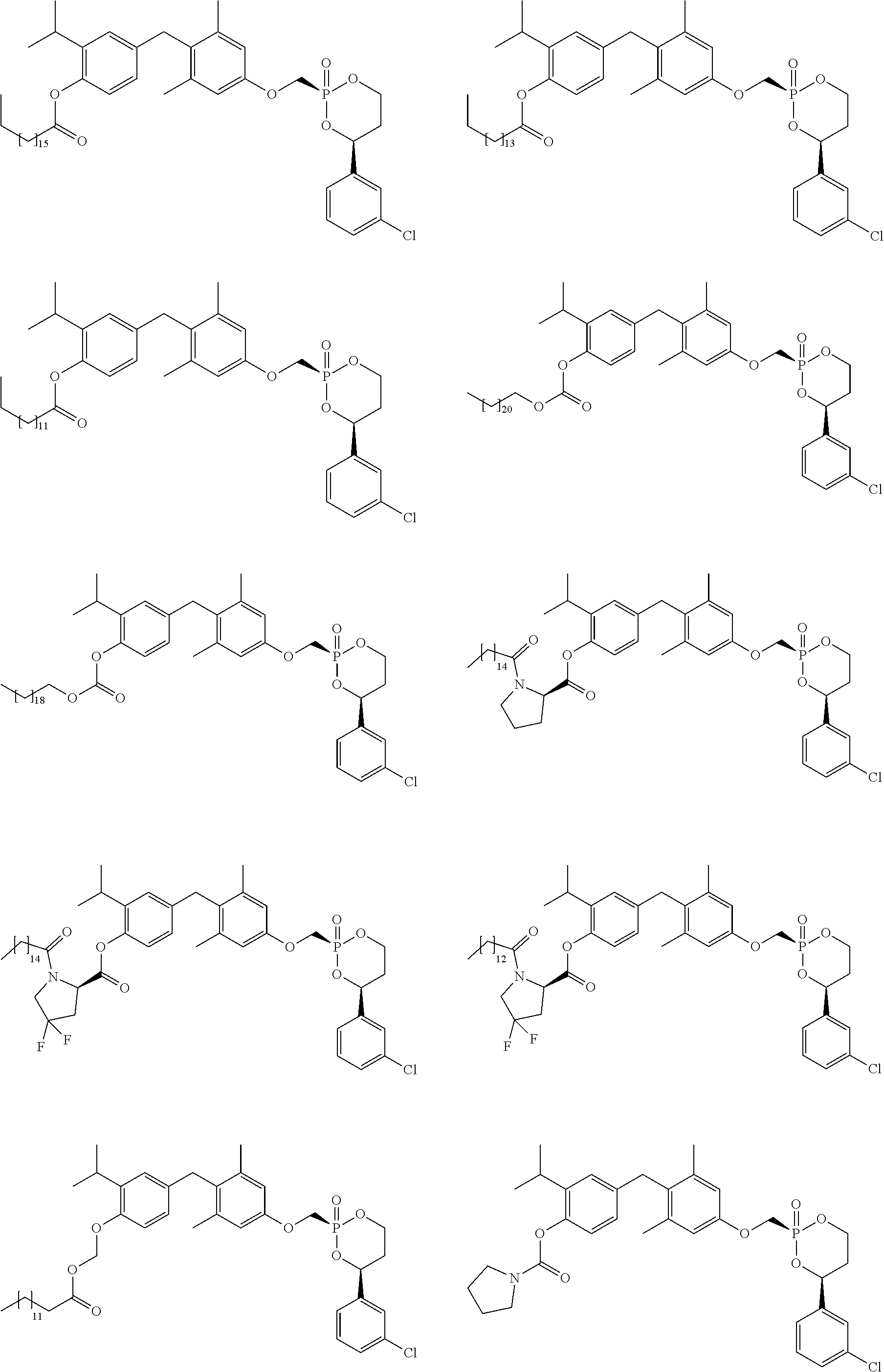

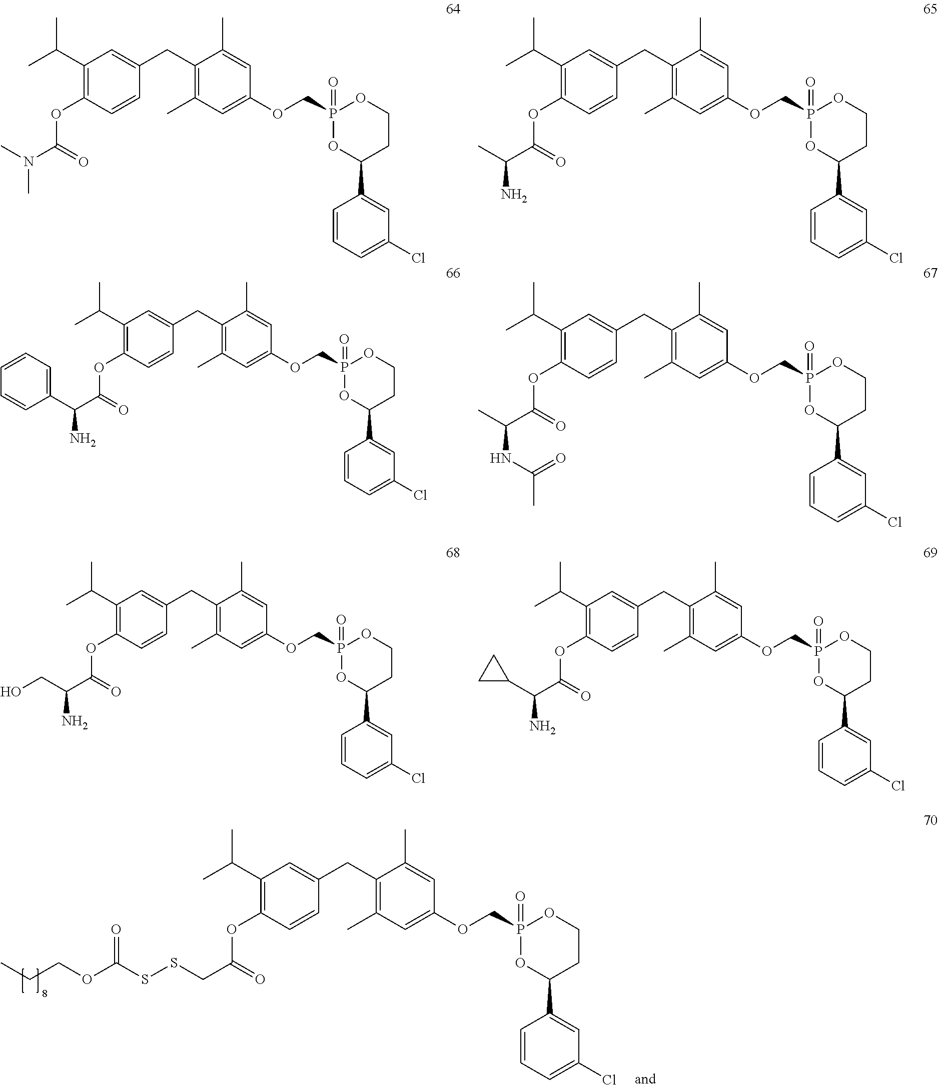

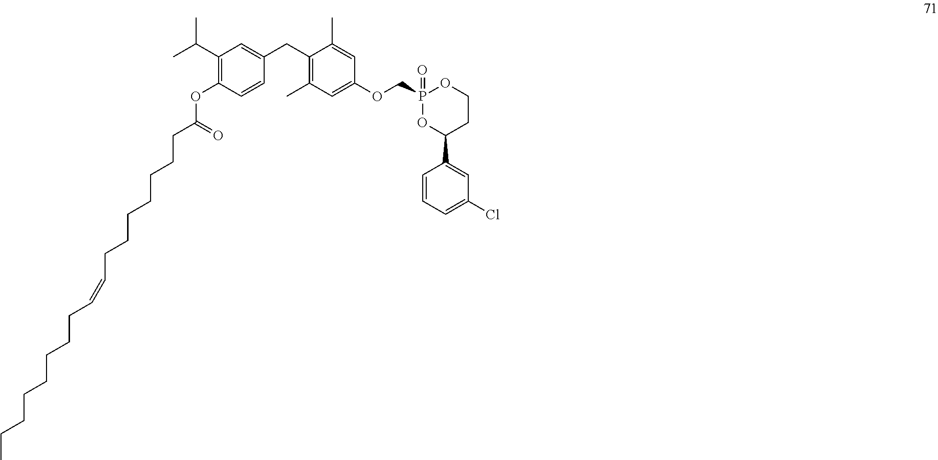 [SMILES: CCCCCCCC/C=C\CCCCCCCC(=O)Oc1ccc(Cc2c(C)cc(OC[P@@]3(=O)OCC[C@@H](c4cccc(Cl)c4)O3)cc2C)cc1C(C)C]

In some embodiments, the formula I is formula III,

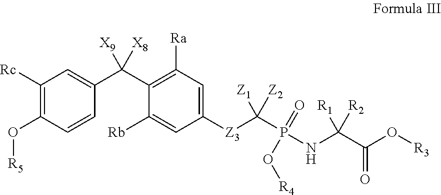

wherein Each of Ra and Rb is independently selected from the group consisting of CH₃, CD₃, Cl, Br, I and CF₃;
each R_{c} is selected from the group consisting of hydrogen, halogen, —CF₃, —OCF₃, cyano, optionally substituted —C₁-C₁₂ alkyl, optionally substituted —C₂-C₁₂ alkenyl, optionally substituted-C₂-C₁₂ alkynyl, optionally substituted —C₀₋₆alkyl-aryl, optionally substituted —C₀₋₆ alkyl-cycloalkyl, optionally substituted —C₀₋₆ alkyl-heterocycloalkyl, optionally substituted —C₃₋₈ cycloalkyl;
wherein R_{c} is optionally substituted with one to ten halogen, H or D;
wherein each of X₈, X₉, Z₁ and Z₂ is independently H, D or halo;
Z₃ is independently O, —CH₂—;
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —CONHR₆, —COOR₆, —CONR₆R₇, —CH₂OCOR₆, and —CH₂OCONHR₆;
   wherein each of R₆ and R₇ is independently C₁₋₃₀ alkyl, C₁₋₃₀ alkenyl, or C₁₋₃₀ alkynyl, wherein the C₁₋₃₀ alkyl, the C₁₋₃₀ alkenyl, or the C₁₋₃₀ alkynyl is optionally substituted with one or more substituents each independently selected from the group consisting of halo, NH₂, NO₂, OH, CN, O—C₁₋₆ alkyl, C₆₋₁₀ aryl, and heteroaryl; or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl.

In some embodiments, wherein Z is O;

In some embodiments, wherein each of Z₁ and Z₂ is H;

In some embodiments, wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl;

In some embodiments, wherein R₃ is selected from the group consisting of C₁₋₃₀ alkyl, and C₅₋₁₀ cycloalkyl;

In some embodiments, R_{c} is independently selected from the group consisting of —C₁-C₆ alkyl, —C₀₋₆ alkyl-aryl, —C₀₋₆ alkyl-cycloalkyl; —C₀₋₆ alkyl-heterocycloalkyl, and —C₃-8 cycloalkyl; wherein the R_{c} is optionally substituted with halo.

In some embodiments, the formula I is formula IV,

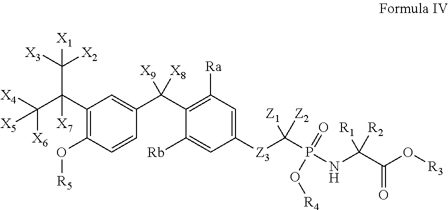

wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —CONHR₆, —COOR₆, —CONR₆R₇, —CH₂OCOR₆, and —CH₂OCONHR₆;
wherein each of X₁, X₂, X₃, X₄, X₅, X₆, X₇, X₈ and X₉ is independently H, D or halo,
wherein each of Z₁ and Z₂ is independently H, D or halo;
wherein each of Ra and Rb is independently selected from the group consisting of CH₃, CD₃, Cl, Br, I or CF₃;
wherein each of R₆ and R₇ is independently C₁₋₃₀ alkyl, C₁₋₃₀ alkenyl, or C₁₋₃₀ alkynyl,
wherein the C₁₋₃₀ alkyl, the C₁₋₃₀ alkenyl, or the C₁₋₃₀ alkynyl is optionally substituted with one or more substituents each independently selected from the group consisting of halo, NH₂, NO₂, OH, CN, O—C₁₋₆ alkyl, C₆₋₁₀ aryl, and heteroaryl; or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl.

In some embodiments, wherein each of Z₁ and Z₂ is H;

In some embodiments, wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl;

In some embodiments, herein R₃ is selected from the group consisting of C₁₋₃₀ alkyl, and C₅₋₁₀ cycloalkyl.

In some embodiments, the formula I is Formula V,

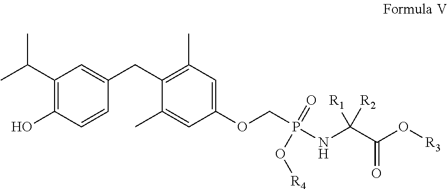

wherein R₁ and R₂ is independently selected from the group consisting of H, C₁₋₆ alkyl, C₆₋₁₀ aryl, heteroaryl, C₁₋₆ alkyl-C₆₋₁₀ aryl, and C₁₋₆ alkyl-heteroaryl;
wherein R₃ is selected from the group consisting of H, C₁₋₃₀ alkyl, and C₅₋₁₀ cycloalkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl;

In some embodiments, wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl;

In some embodiments, herein R₃ is selected from the group consisting of C₁₋₃₀ alkyl, and C₅₋₁₀ cycloalkyl.

In some embodiments, the formula I is formula VI,

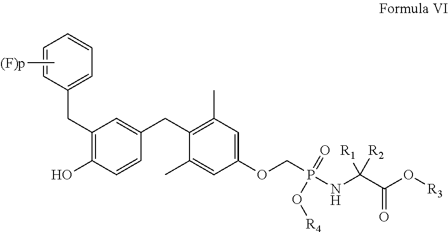

wherein R₁ and R₂ is independently selected from the group consisting of H, C₁₋₆ alkyl, C₆₋₁₀ aryl, heteroaryl, C₁₋₆ alkyl-C₆₋₁₀ aryl, and C₁₋₆ alkyl-heteroaryl; wherein each of the alkyl, aryl and heteroaryl is optionally substituted with halogen, C₁₋₆ alkyl and C₁₋₆ haloalkyl.
wherein R₃ is selected from the group consisting of H, C₁₋₃₀ alkyl, and C₅₋₁₀ cycloalkyl.
wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl;
p is an integer from 0-3.

In some embodiments, wherein the R₃ is C₁₋₆ alkyl, or C₅₋₁₀ cycloalkyl;

In some embodiments wherein the R₃ is C₇₋₃₀ alkyl, or C₅₋₁₀ cycloalkyl;

In some embodiments, wherein the R₄ is C₆₋₁₀ aryl; and C₆₋₁₀ the aryl is optionally substituted with halogen, C₁₋₆ alkyl;

In some embodiments, the compound of the present application is selected from the group consisting of:

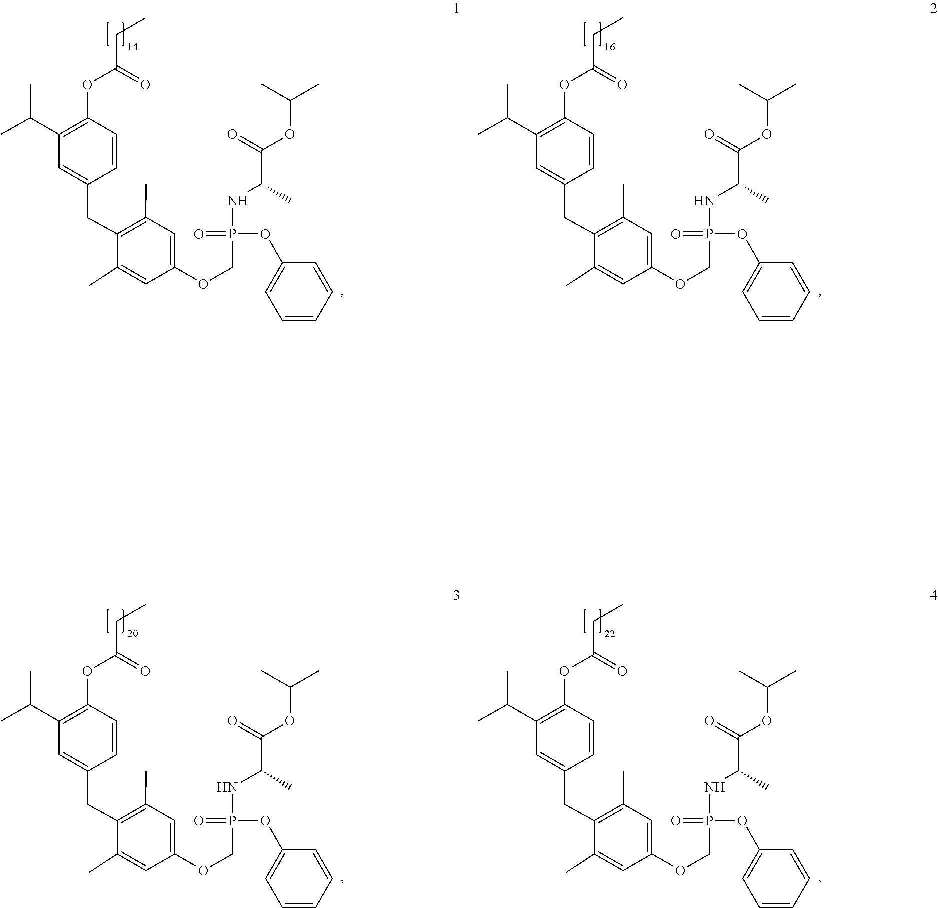

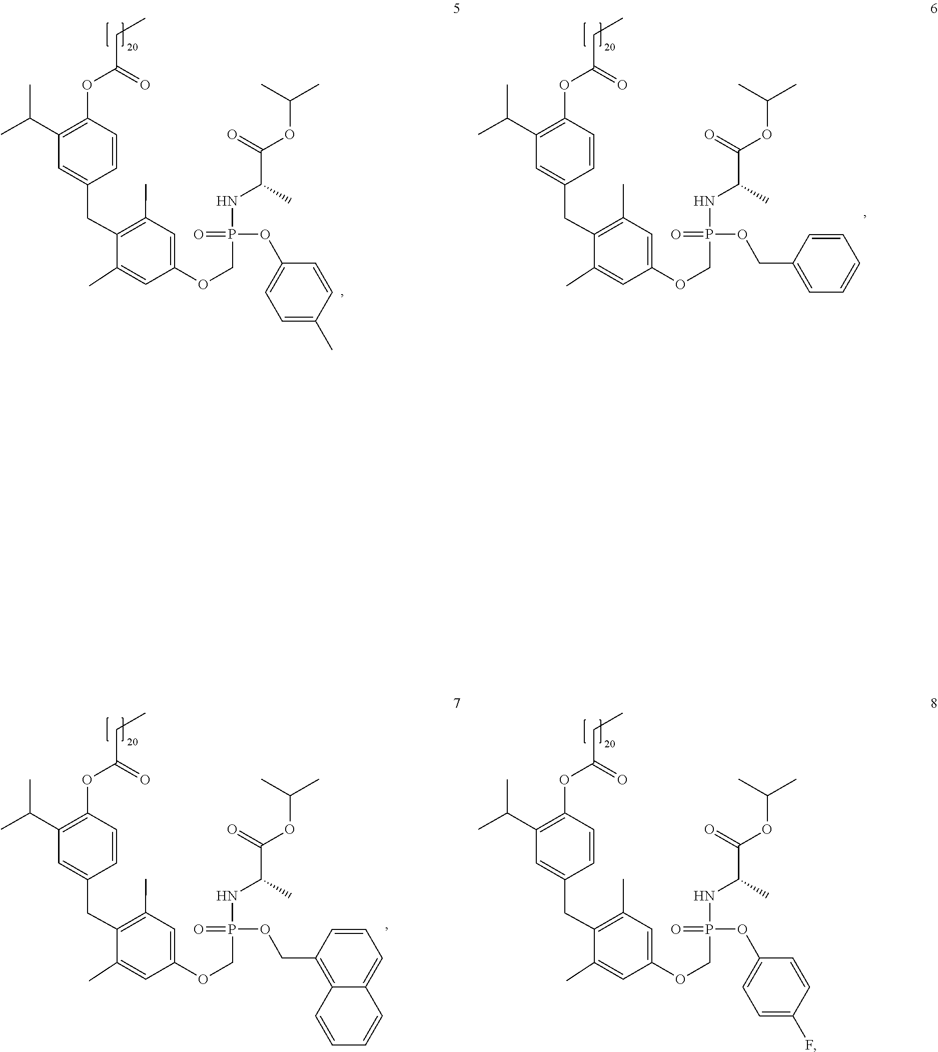

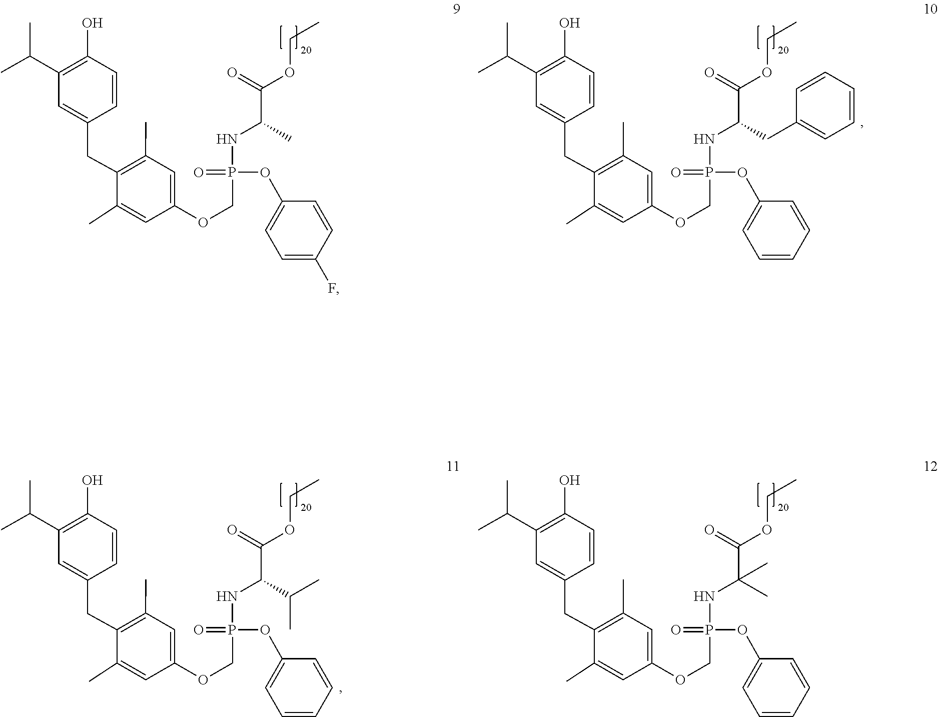

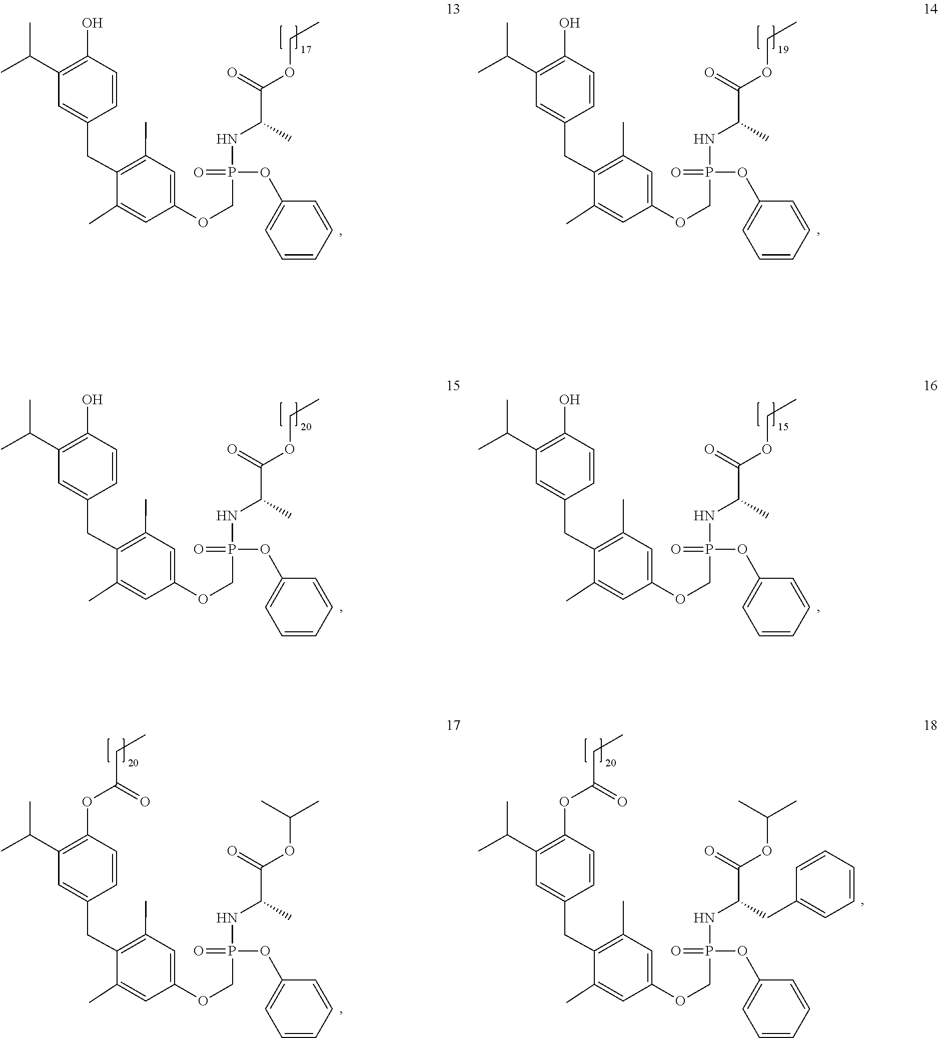

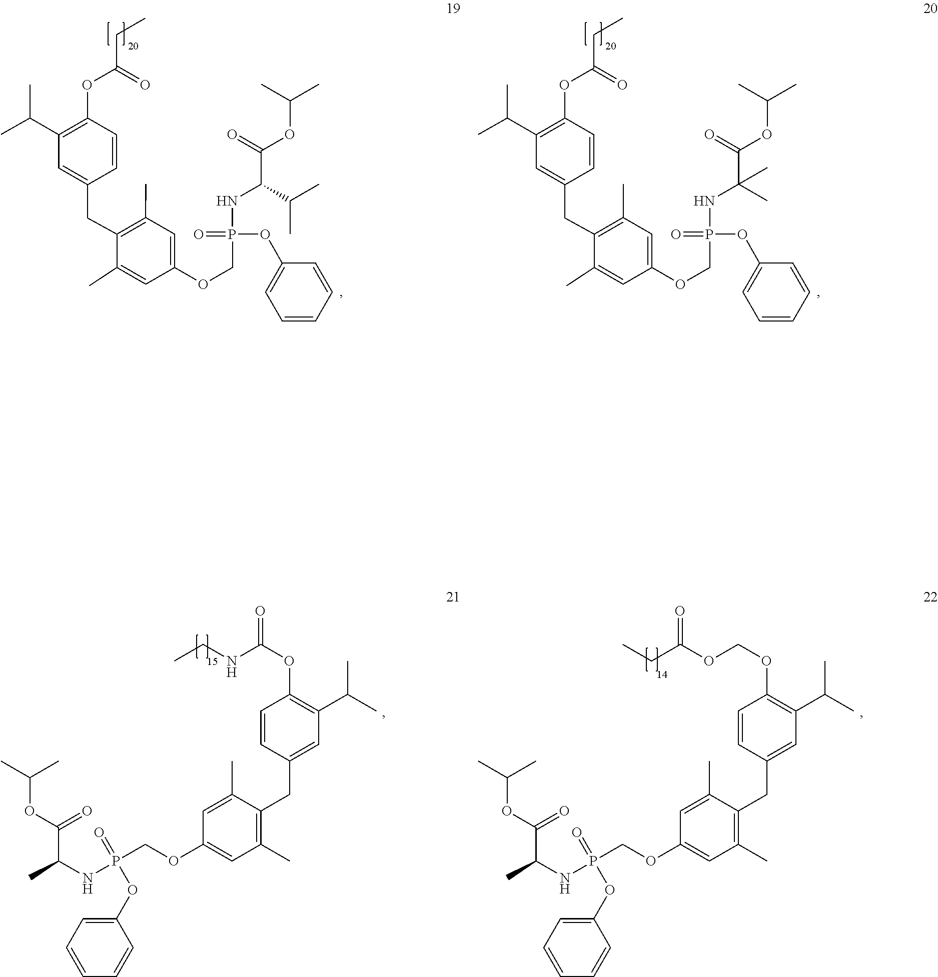

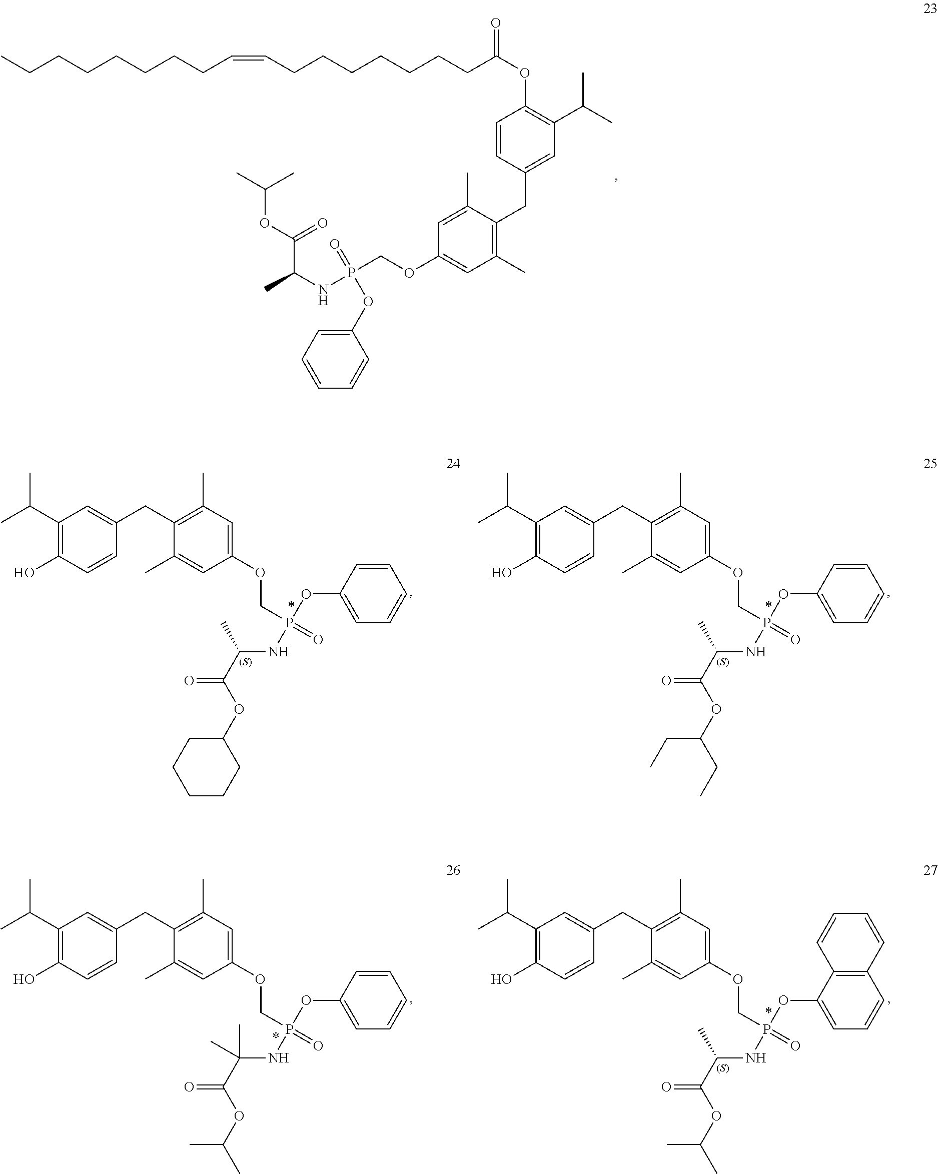

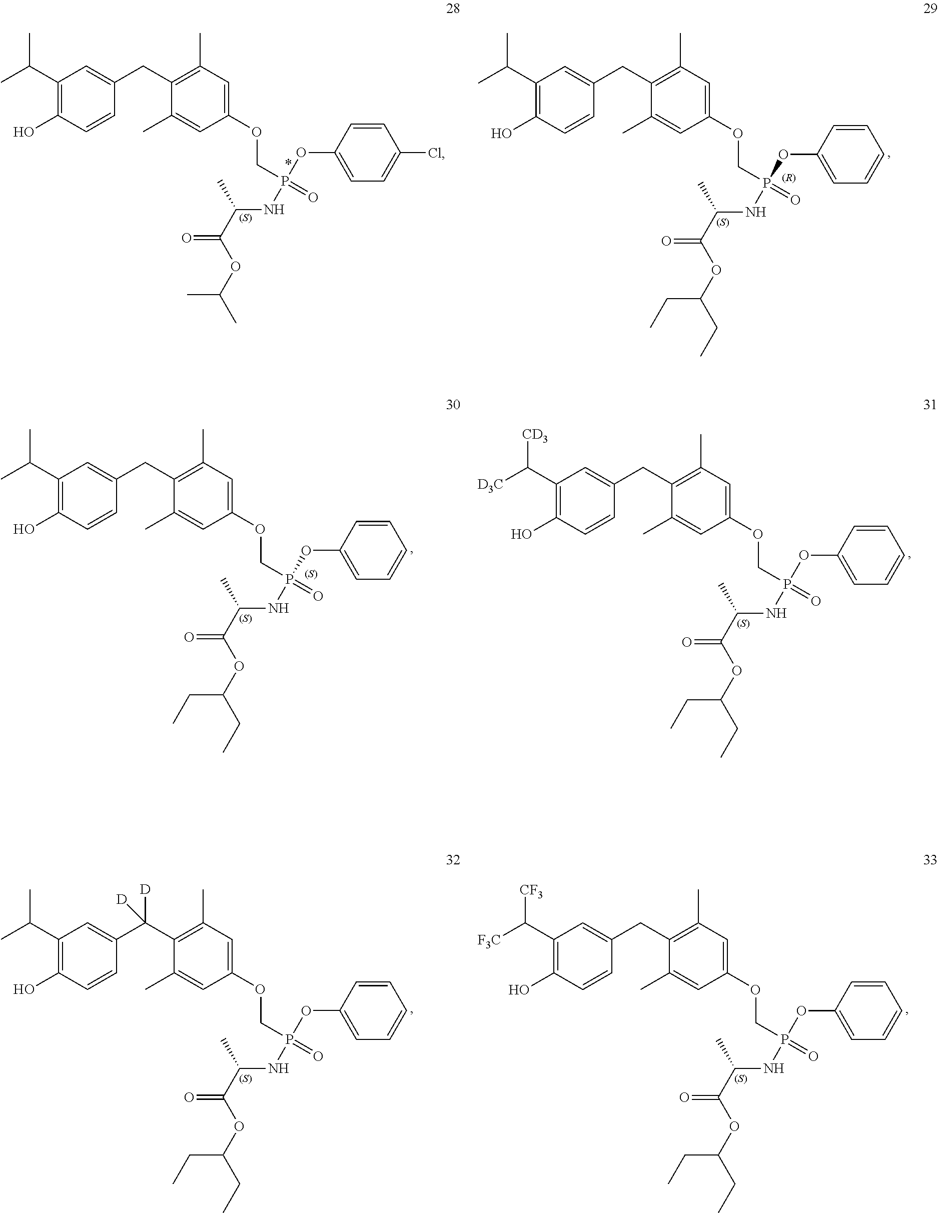

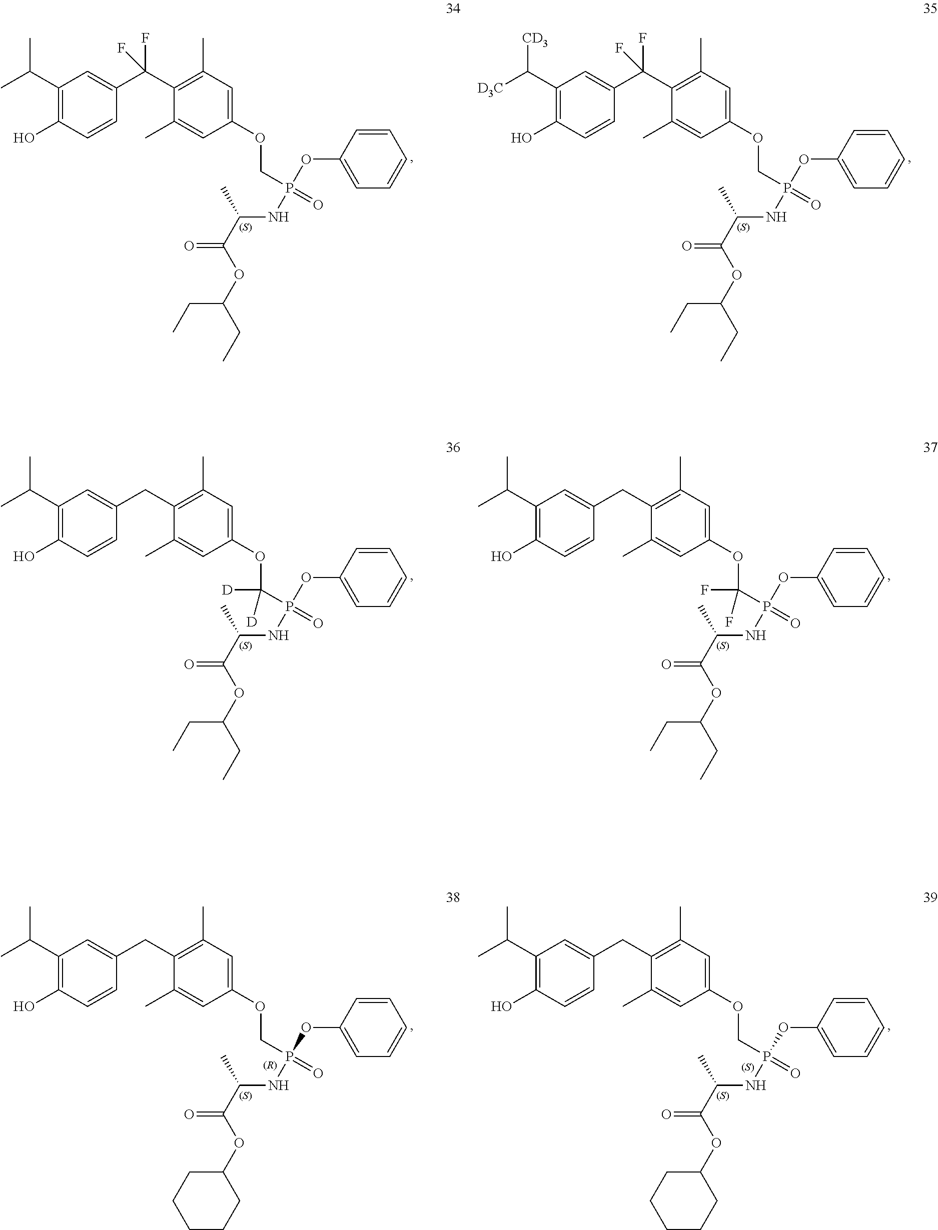

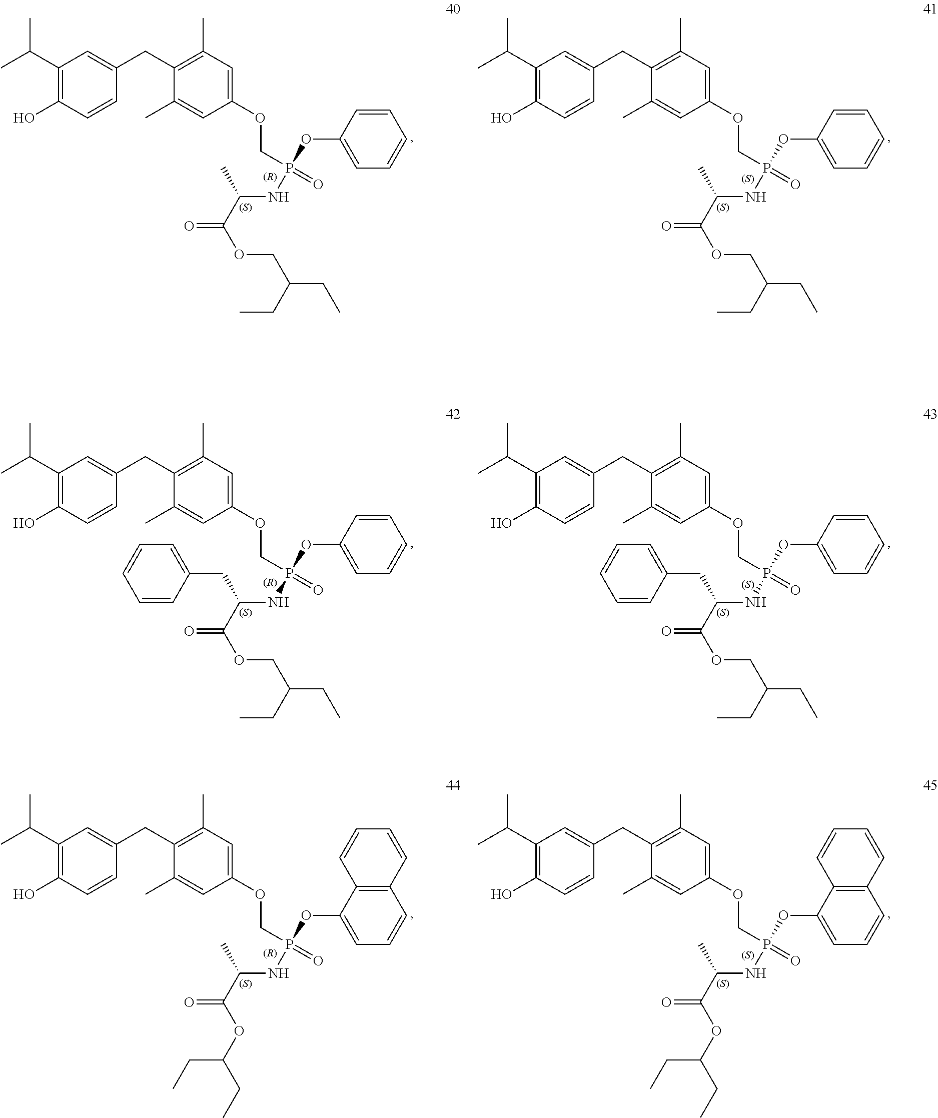

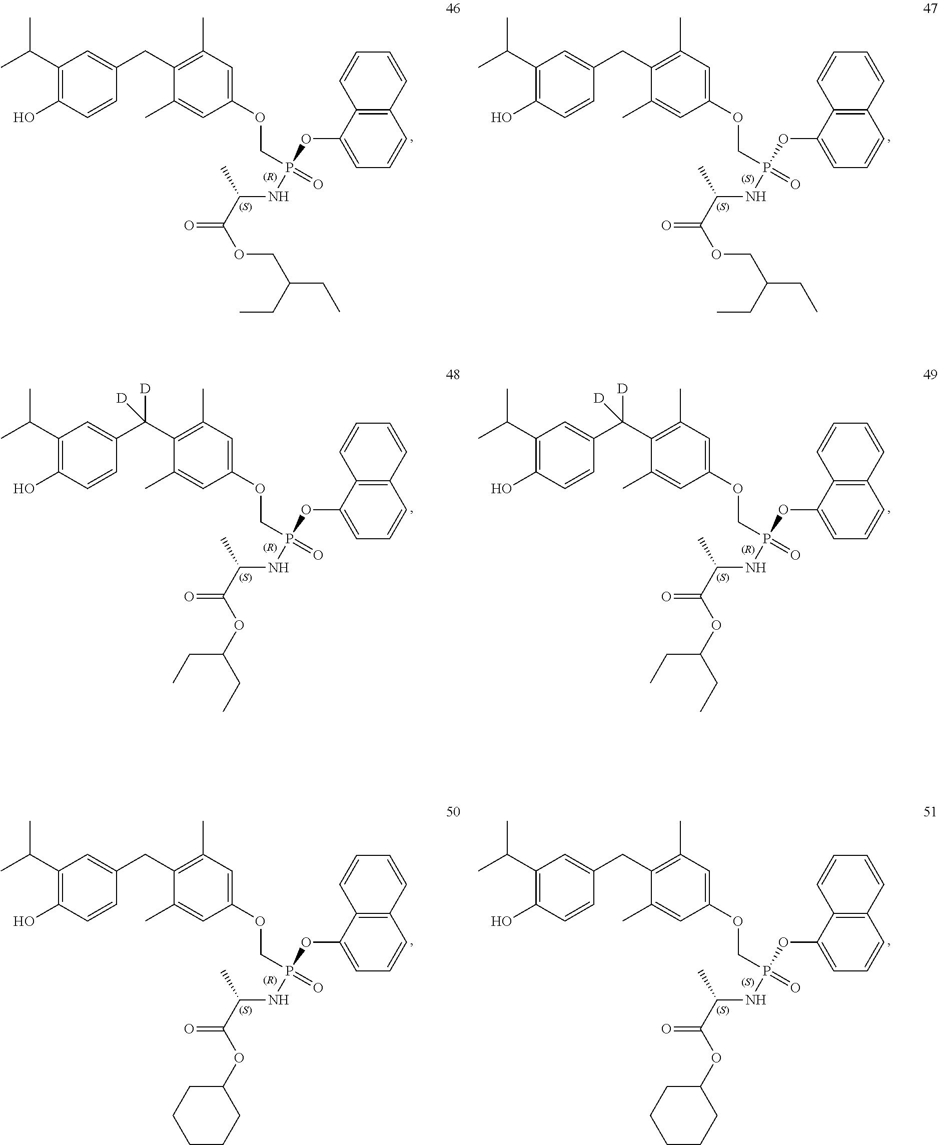

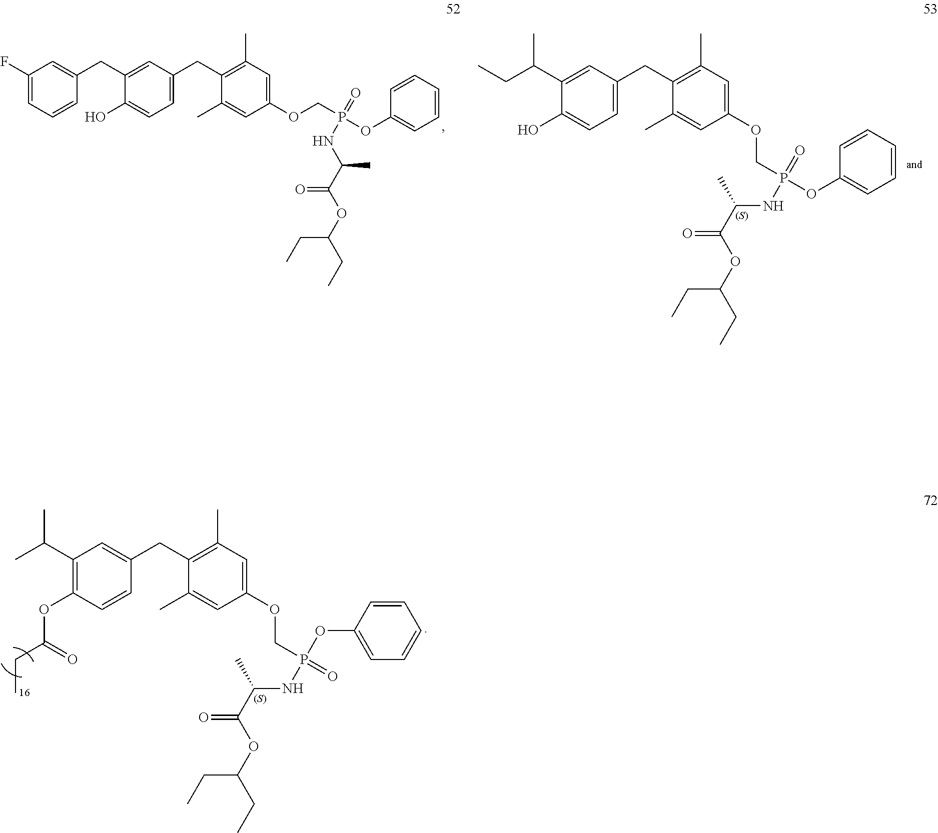

In some embodiments, the compound of the formula I has the following formula

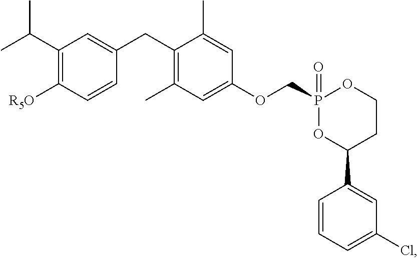

stereoisomers thereof, pharmaceutically acceptable salts thereof, and deuterium substitutes thereof,
wherein R₅ is selected from the group consisting of —C(O)R₆, —C(O)OR₆, —C(O)NR₇R₁₀, CR₁₂R₁₃OC(O)R₇, CR₁₂R₁₃OC(O)OR₇, and

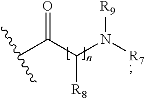

wherein R₆ is selected from the group consisting of C₁₃₋₃₀ alkyl, C₁₃₋₃₀ alkenyl, and C₁₃₋₃₀ alkynyl, wherein each of the C₁₃₋₃₀ alkyl, C₁₃₋₃₀ alkenyl, and C₁₃₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₇ is selected from the group consisting of H, —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl, wherein each of the —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NR(C₁₋₃₀ alkyl, —C(O) NRCC₂₋₃₀ alkenyl, and —C(O) NRCC₂₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₁₀ is selected from the group consisting of H, C₁₋₆ alkyl, C₂₋₆ alkenyl, C₂₋₆ alkynyl, —(CH₂), C₃₋₁₀cycloalkyl, —(CH₂)ₙ aryl, and —(CH₂), heteroaryl, wherein each of the C₁₋₆ alkyl, C₂₋₆ alkenyl, C₂₋₆ alkynyl, —(CH₂), C₃₋₁₀cycloalkyl, —(CH₂)ₙ aryl, and —(CH₂)ₙ heteroaryl is optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein each of R₁₂ and R₁₃ is independently selected from the group consisting of H, deuterium, C₁₋₆ alkyl, halo, and C₁₋₆ haloalkyl, wherein each of the C₁₋₆ alkyl, halo and C₁₋₆ haloalkyl can be optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein each of R₈ and R₉ is independently selected from the group consisting of H, OH, C₁₋₆ alkyl, halo, —O—C₁₋₃ alkyl, —S—C₁₋₃ alkyl, —C₃₋₁₀ cycloalkyl, 3-10 membered heterocyclyl, aryl, and 5-10 membered heteroaryl, wherein each of the C₁₋₆ alkyl, —O—C₁₋₃ alkyl, —S—C₁₋₃ alkyl, —C₃₋₁₀ cycloalkyl, 3-10 membered heterocyclyl, aryl, and 5-10 membered heteroaryl can be optionally substituted with one or more substituents each independently selected from the group consisting of OH, halo, —O—C₁₋₃ alkyl, —S—C₁₋₃ alkyl, —C₃₋₁₀ cycloalkyl, 3-10 membered heterocyclyl, aryl, and 5-10 membered heteroaryl;
or wherein R₈ and R₉ are combined with the atoms to which they are attached to form a 3-10 membered heterocyclyl, which is optionally substituted with one to four R₆, wherein each R₆ is independently H, halo, C₁₋₆ alkyl, C₁₋₆ haloalkyl, or alkoxy;
wherein n=0, 1, 2, or 3;
wherein each 5-10 membered heteroaryl mentioned above has one to four heteroatoms, each is independently N, O, or S; and
wherein each 3-10 membered heterocyclyl mentioned above has one to four heteroatoms, each is independently N, O, or S.

In some embodiments, the compound of the formula I has the following formula:

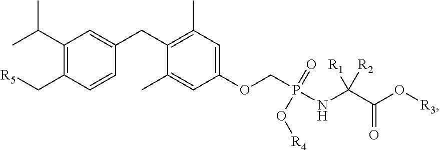

stereoisomers thereof, pharmaceutically acceptable salts thereof, deuterium substitutes thereof, isomers thereof, prodrugs thereof, and metabolites thereof, wherein
each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form C₃₋₁₀ cycloalkyl or heterocyclyl;
R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
R₅ is selected from the group consisting of H, —COR₆, —CONHR₆, —COOR₆, —CONR₆R₇, —CH₂OCOR₆, and —CH₂OCONHR₆;
wherein each of R₆ and R₇ is independently C₁₋₃₀ alkyl, C₁₋₃₀ alkenyl, or C₁₋₃₀ alkynyl, wherein the C₁₋₃₀ alkyl, C₁₋₃₀ alkenyl, or C₁₋₃₀ alkynyl is optionally substituted with one or more substituents each independently selected from the group consisting of halo, NH₂, NO₂, OH, CN, O—C₁₋₆ alkyl, C₆₋₁₀ aryl, and heteroaryl; or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl.

In some embodiments, the compound of the formula I has the following formula:

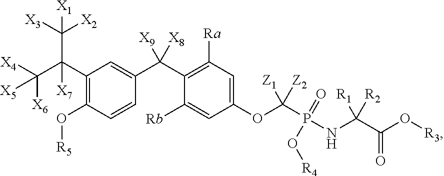

stereoisomers thereof, pharmaceutically acceptable salts thereof, deuterium substitutes thereof, isomers thereof, prodrugs thereof, and metabolites thereof,
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —CONHR₆, —COOR₆, —CONR₆R₇, —CH₂OCOR₆, and —CH₂OCONHR₆;
wherein each of X₁, X₂, X₃, X₄, X₅, X₆, X₇, X₈ and X₉ is independently H, D or F;
wherein each of Z₁ and Z₂ is independently H or D;
wherein each of Ra and Rb is independently selected from the group consisting of CH₃, CD₃, Cl, Br, I or CF₃;
wherein each of R₆ and R₇ is independently C₁₋₃₀ alkyl, C₁₋₃₀ alkenyl, or C₁₋₃₀ alkynyl, wherein the C₁₋₃₀ alkyl, the C₁₋₃₀ alkenyl, or the C₁₋₃₀ alkynyl is optionally substituted with one or more substituents each independently selected from the group consisting of halo, NH₂, NO₂, OH, CN, O—C₁₋₆ alkyl, C₆₋₁₀ aryl, and heteroaryl; or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl.

Another aspect of the present application relates to a pharmaceutical composition that comprises (1) a compound of Formula I, a stereoisomer thereof, a pharmaceutically acceptable salt thereof, a deuterated compound thereof, or a hydrate or solvate thereof, and (2) a pharmaceutically acceptable carrier.

### III. Methods of Treatment

Another aspect of the preset application relates to a method for preventing, treating or ameliorating a symptom of a disease or condition in a subject. The method comprises the step of administering to the subject an effective amount of the pharmaceutical composition of the present application. Examples of the disease or condition include, but are not limited to, obesity, hyperlipidemia, hypercholesterolemia, diabetes mellitus, nonalcoholic steatohepatitis (NASH), hepatic steatosis, arteriosclerosis, cardiovascular disease, hypothyroidism, and thyroid cancer.

The present application also relates to formula (I) compounds or the stereoisomers thereof, pharmaceutically acceptable salts thereof, deuterated compounds thereof, hydrates thereof or solvates thereof, in the preparation of medicaments for the treatment and/or prevention of obesity, hyperlipidemia, hypercholesterolemia, diabetes mellitus, nonalcoholic steatohepatitis (NASH), hepatic steatosis, arteriosclerosis, cardiovascular disease, hypothyroidism, or thyroid cancer.

### EXAMPLES

### Synthesis

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of typical compounds of Formula I, or a pharmaceutically acceptable salt thereof, e.g., compounds having structures described by one or more of Formula I, or other formulas or compounds disclosed herein, may be accomplished as described in the following examples.

### General Syntheses

Typical embodiments of compounds in accordance with the present application may be synthesized using the general reaction schemes and/or examples described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Starting materials are typically obtained from commercial sources or synthesized using published methods for synthesizing compounds which are embodiments of the present application, inspection of the structure of the compound to be synthesized will provide the identity of each substituent group The identity of the final product will generally render apparent the identity of the necessary starting materials by a simple process of inspection, given the examples herein. Group labels (e.g., R₁, R₂) used in the reaction schemes herein are for illustrative purposes only and unless otherwise specified do not necessarily match by name or function the labels used elsewhere to describe compounds of Formula I or aspects or fragments thereof.

### Synthetic Reaction Parameters

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of the present application may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), N, N-dimethylformamide (“DMF”), chloroform, methylene chloride (or dichloromethane), diethyl ether, methanol, pyridine and the like). Unless specified to the contrary, the solvents used in the reactions of the present application are inert organic solvents, and the reactions are carried out under an inert gas, preferably nitrogen.

The term “q.s.” means adding a quantity sufficient to achieve a stated function, e.g., to bring a solution to the desired volume (i.e., 100%).

Compounds as provided herein may be synthesized according to the general schemes provided below. In the Schemes below, it should be appreciated that each of the compounds shown therein may have protecting groups as required present at any step. Standard protecting groups are well within the purview of one skilled in the art.

### Preparation Example 1: Preparation of Compound 1 4-(4-(((((S)-1-isopropoxy-1-oxopropan-2-yl)amino)(phenoxy)phosphoryl)methoxy)-2,6-dimethylbenzyl)-2-isopropylphenyl pentadecanoate (compound 1)

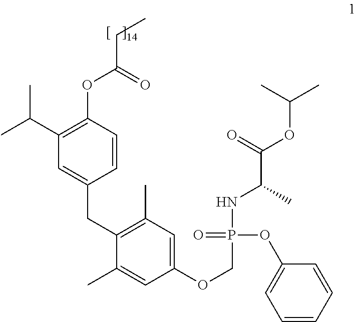

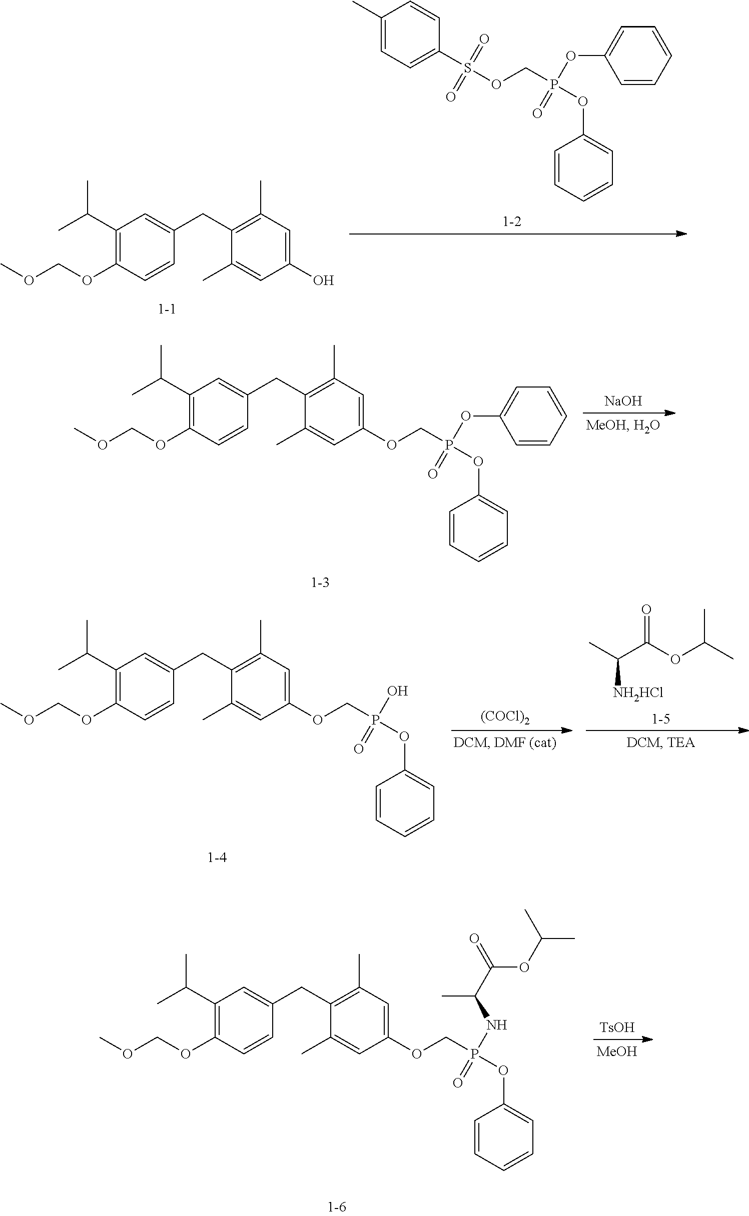

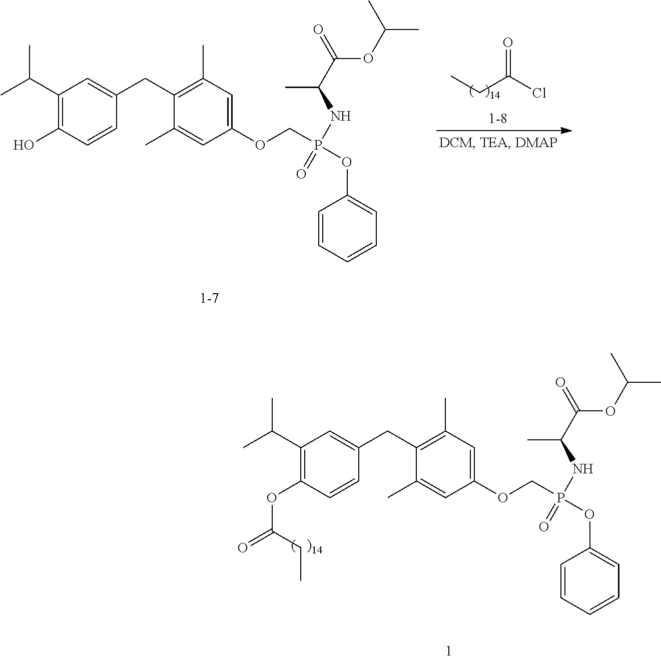

### (1) Synthesis of Intermediate 1-3:

NaH (153 mg, 3.816 mmol) was added to a solution of 1-1 (1 g, 3.18 mmol) in THF (10 ml) at 0° C. and stirred for 0.5 h, then a solution of 1-2 (1.60 g, 3.816 mmol) in THF (10 ml) was added to above reaction. The mixture was stirred for 2 h at rt. The reaction was quenched by H₂O, extracted with EA. The organic phase was concentrated in vacuo and purified by silica to obtain intermediate 1-3 (1.0 g, yield 56%).

### (2) Synthesis of Intermediate 1-4:

1N NaOH (5 ml) was added to a solution of 1-3 (1 g, 1.78 mmol) in MeOH (10 ml) and stirred for 16 h at room temperature (RT). The reaction was extracted with EA. The organic phase was concentrated in vacuo and purified by silica to obtain intermediate 1-4 (0.56 g, yield 65%).

### (3) Synthesis of Intermediate 1-6:

A reaction mixture of 1-4 (0.56 g, 1.15 mmol), (COCl)₂ (0.439 g, 3.46 mmol) and DMF (cat) in DCM (10 ml) was stirred for 1 h at RT. The reaction was concentrated in vacuo, then added DCM (10 ml), TEA (0.929 g, 9.2 mmol), DAMP (14 mg, 0.115 mmol) and 1-5 (0.384 g, 2.3 mmol) The reaction was stirred for 2 h at RT, quenched by H₂O, extracted with DCM. The organic phase was concentrated in vacuo and purified by silica to obtain intermediate 1-6. (0.35 g, yield 50%).

### (4) Synthesis of Intermediate 1-7:

A reaction mixture of 1-6 (0.35 g, 0.58 mmol) and TsOH (0.3 g, 1.74 mmol) in MeOH (5 ml) was stirred for 4 h at 50° C. The reaction was quenched by H₂O, and extracted with DCM. The organic phase was concentrated in vacuo and purified by silica to obtain intermediate 1-7 (0.21 g, yield 65%).

### (5) Synthesis of Compound 1:

A reaction mixture of 1-7 (0.21 g, 0.38 mmol), TEA (77 mg, 0.76 mmol), DAMP (5 mg, 0.038 mmol) and 1-8 (0.114 g, 0.418 mmol) in DCM (5 ml) was stirred for 2 h at RT, then quenched by H₂O, extracted with DCM. The organic phase was concentrated in vacuo and purified by silica to obtain compound 1 (150 mg, yield 50%).

### Preparation Example 2: Synthesis of Compound 6 4-(4-(((benzyloxy)(((S)-1-isopropoxy-1-oxopropan-2-yl)amino)phosphoryl)methoxy)-2,6-dimethylbenzyl)-2-isopropylphenyl docosanoate (compound 6)

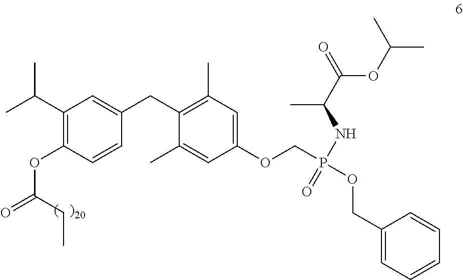

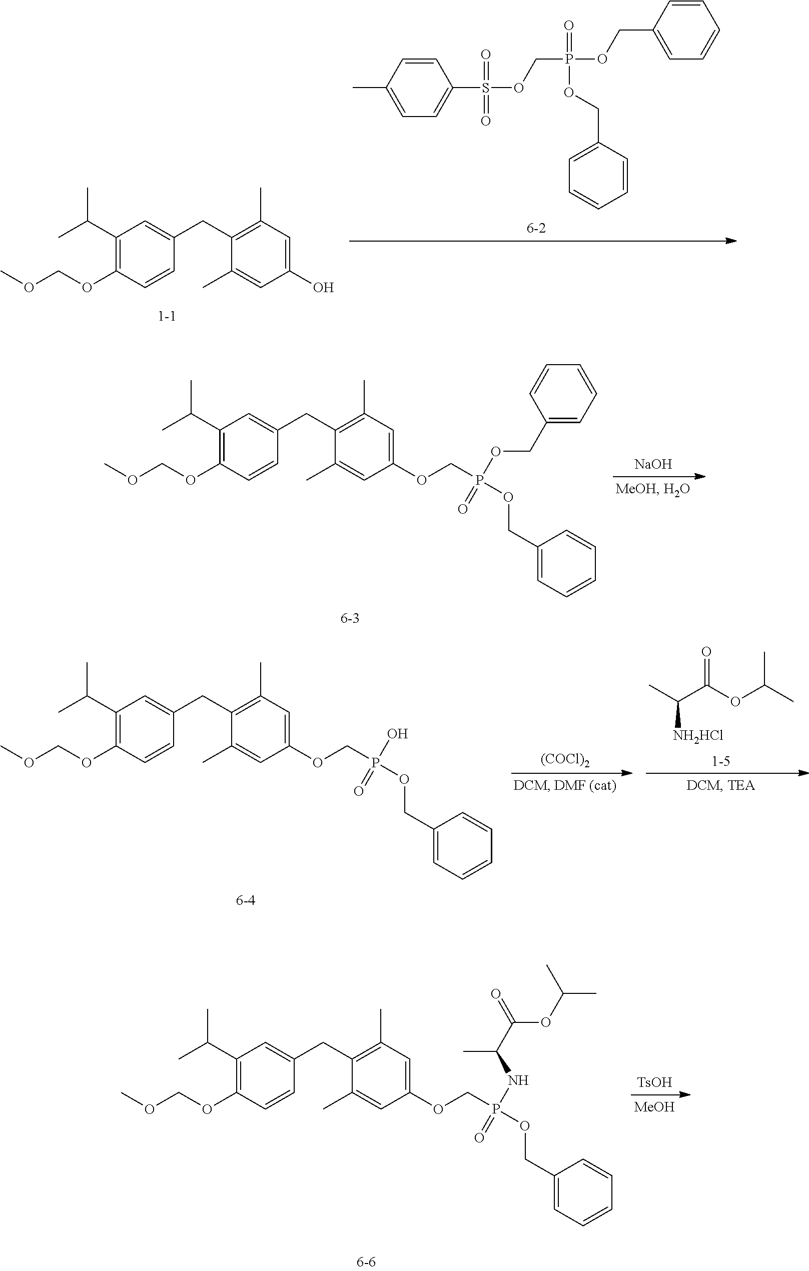

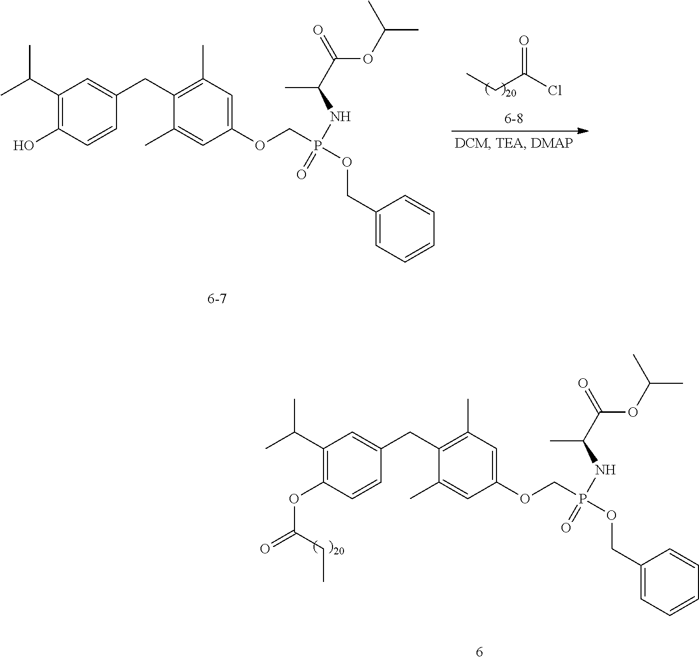

### (1) Synthesis of Intermediate 6-3:

NaH (153 mg, 3.816 mmol) was added to a solution of 1-1 (1 g, 3.18 mmol) in THF (10 ml) at 0° C. and stirred for 0.5 h, then a solution of 6-2 (1.7 g, 3.816 mmol, refer to Example 4) in THF (10 ml) was added to above reaction. The mixture was stirred for 2 h at RT. The reaction was quenched by H₂O, extracted with EA. The organic phase was concentrated in vacuo and purified by silica to obtain intermediate 6-3 (1.2 g, yield 64%).

### (2) Synthesis of Intermediate 6-4:

1N NaOH (5 ml) was added to a solution of 6-3 (1.2 g, 2.03 mmol) in MeOH (10 ml) and stirred for 16 h at RT. The reaction was extracted with EA. The organic phase was concentrated in vacuo and purified by silica to obtain intermediate 6-4 (0.50 g, yield 49%).

### (3) Synthesis of Intermediate 6-6:

A reaction mixture of 6-4 (0.50 g, 1.0 mmol), (COCl)₂ (0.254 g, 2 mmol) and DMF (cat) in DCM (10 ml) was stirred for 1 h at RT. The reaction was concentrated in vacuo, then DCM (10 ml), TEA (0.929 g, 9.2 mmol), DAMP (14 mg, 0.115 mmol) and 1-5 (0.384 g, 2.3 mmol) were added and stirred for 2 h at RT. The reaction was quenched by H₂O, and extracted with DCM. The organic phase was concentrated in vacuo and purified by silica to obtain intermediate 6-6 (0.27 g, yield 44%).

### (4) Synthesis of Intermediate 6-7:

A solution of 6-6 (0.27 g, 0.44 mmol), TsOH (0.151 g, 0.88 mmol) in MeOH (5 ml) was stirred for 4 h at 50° C. The reaction was quenched by H₂O, and extracted with DCM. The organic phase was concentrated in vacuo and purified by silica to obtain intermediate 6-7 (0.15 g, yield 60%).

### (5) Synthesis of Compound 6:

A solution of 6-7 (0.15 g, 0.26 mmol), TEA (55 mg, 0.53 mmol), DAMP (5 mg, 0.028 mmol) and 6-8 (0.112 g, 0.418 mmol) in DCM (5 ml) was stirred for 2 h at RT. The reaction was then quenched by H₂O, and extracted with DCM. The organic phase was concentrated in vacuo and purified by silica to obtain compound 6 (100 mg, yield 43%).

### Preparation Example 3: Synthesis of Compound 13 octadecyl (((4-(4-hydroxy-3-isopropylbenzyl)-3,5-dimethylphenoxy)methyl)(phenoxy)phosphoryl)-L-alaninate (compound 13)

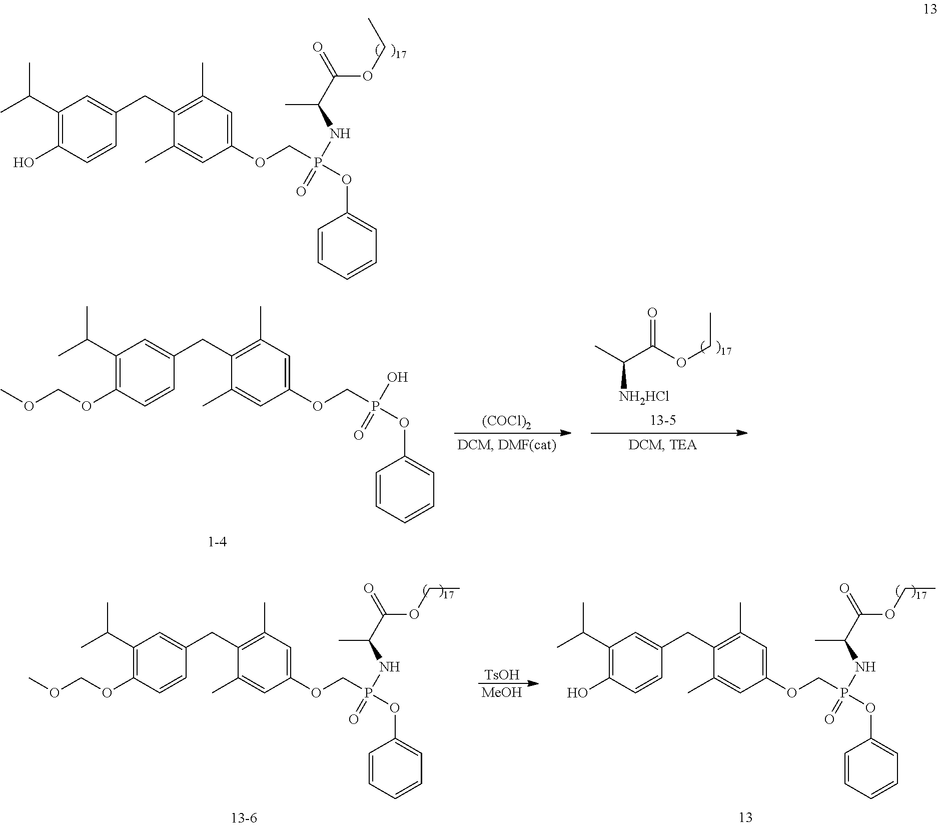

### (1) Synthesis of Intermediate 13-6:

The solution of 1-4 (0.50 g, 1.0 mmol), (COCl)₂ (0.254 g, 2 mmol) and DMF (cat) in DCM (10 ml) was stirred for 1 h at RT. The reaction was concentrated in vacuo, then DCM (10 ml), TEA (0.929 g, 9.2 mmol), DAMP (14 mg, 0.115 mmol) and 13-5 (0.756 g, 2.0 mmol) were added and stirred for 2 h at RT. The reaction was quenched by H₂O, and extracted with DCM. The organic phase was concentrated in vacuo and purified by silica to obtain intermediate 13-6 (0.30 g, yield 37%).

### (3) Synthesis of Compound 13:

The solution of 13-6 (0.30 g, 0.37 mmol) and TsOH (0.128 g, 0.75 mmol) in MeOH (5 ml) was stirred for 4 h at 50° C. The reaction was quenched by H₂O, and extracted with DCM. The organic phase was concentrated in vacuo and purified by silica to give the compound 13 (0.17 g, yield 60%).

### Preparation Example 4: Synthesis of Intermediate 1-2 (diphenoxyphosphoryl)methyl 4-methylbenzenesulfonate (intermediate 1-2)

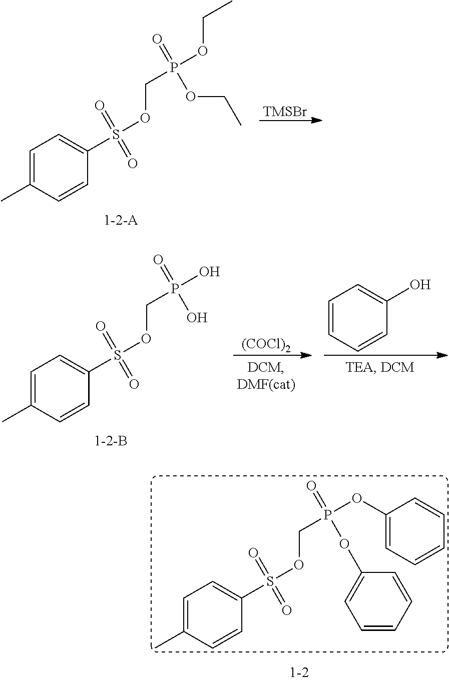

### (1) Synthesis of Intermediate 1-2-B:

A solution of 1-2-A (1 g, 3.1 mmol) and TMSBr (3.3 g, 21.7 mmol) in DCM (10 ml) was stirred for 16 h at RT. The reaction was concentrated in vacuo. MTBE and 2-3N NaOH were added and extracted with MTBE. The pH of the aqueous phase was adjusted to pH=1-2 with 3N HCl. After extraction with EA, the organic phase was concentrated in vacuo to obtain intermediate 1-2-B (0.6 g, yield 72%).

### (2) Synthesis of Intermediate 1-2:

The solution of intermediate 1-2-B (0.6 g, 2.25 mmol), (COCl)₂ (0.856 g, 6.75 mmol) and DMF (cat) in DCM (10 ml) was stirred for 1 h at RT. The reaction mixture was concentrated in vacuo. DCM (10 ml), TEA (1.36 g, 13.5 mmol), DAMP (27 mg, 0.225 mmol) and phenol (0.528 g, 5.625 mmol) were then added to the reaction mixture and stirred for 2 h at RT. The reaction was quenched by H₂O, and extracted with DCM. The organic phase was concentrated in vacuo and purified by silica to obtain intermediate 1-2 (0.36 g, yield 38%).

### Preparation Example 5: amino-acid ester (Int A)

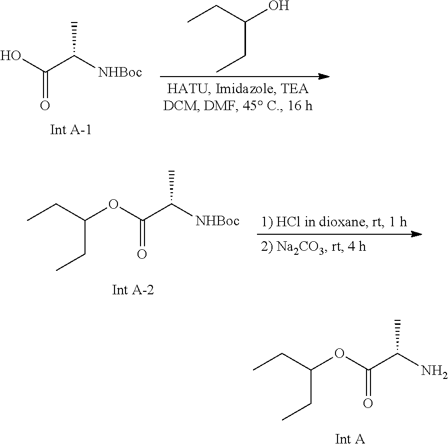

Pentan-3-ol (28 g, 318 mmol) was added to a solution of Int A-1 (60 g, 317 mmol), Imidazole (21 g, 323 mmol), HATU (180 g, 473 mmol) and TEA (64 g, 633 mmol) in DMF/DCM (500 ml/500 ml), and stirred overnight at RT. The reaction was concentrated in vacuo to remove the DCM. The residue was added into water and stirred for 1 h. The mixture was filtrated to obtain a solid. The solid was dried to obtain Int A-2 (80.0 g, yield 97.3%).

The Int A-2 (50 g, 193 mmol) was dissolved into the solution of HCl in dioxane (4 M, 500 ml) at 0-5° C., and stirred for 1 h. The reaction was concentrated in vacuo to obtain a solid. The solid was added into DCM (500 ml) and saturated sodium carbonate solution (500 ml), and stirred for 15 mins. The organic layer was separated and concentrated in vacuo to obtain Int A(27.0 g, yield 87.9%).

### Preparation Example 6: (4-hydroxy-2,6-dimethylphenyl)(3-isopropyl-4-(methoxymethoxy)phenyl)methanone (Int B)

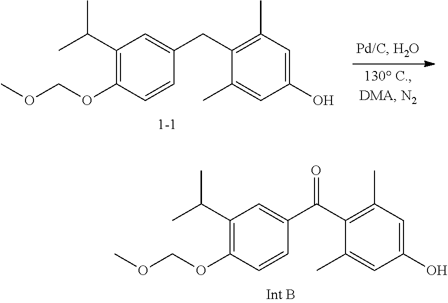 [SMILES: COCOc1ccc(Cc2c(C)cc(O)cc2C)cc1C(C)C ; COCOc1ccc(C(=O)c2c(C)cc(O)cc2C)cc1C(C)C]

A solution of Compound 1-1 (1.0 g, 3.18 mmol), Pd/C (0.05 g, 30 wt %), H₂O (0.2 ml) in DMA (2 ml) was stirred for 48 h at 130-135° C. under N₂. After cooling, the mixture solution was filtrated to get a filtrate. The filtrate was added into ethyl acetate (15 ml) and washed with brine, then concentrated in vacuo and purified by silica to obtain Int B (0.35 g, yield 33.5%).

### Preparation Example 7: 4-((3-isopropyl-4-(methoxymethoxy)phenyl)methyl-d2)-3,5-dimethylphenol (Int C)

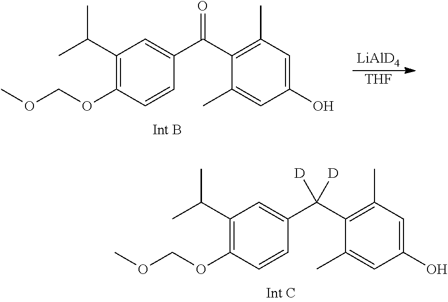 [SMILES: COCOc1ccc(C(=O)c2c(C)cc(O)cc2C)cc1C(C)C ; "LiAlD4" ; [2H]C([2H])(c1ccc(OCOC)c(C(C)C)c1)c1c(C)cc(O)cc1C ; "THF"]

LiAlD4 (25.6 mg, 0.6 mmol) was added to a solution of Int B (0.1 g, 0.3 mmol) in THF (1 ml) at 0° C. and stirred for 1 h. The mixture reaction was quenched with saturated NH₄Cl solution, then EtOAc (20 ml) was added into the mixture reaction. The organic phase was separated and washed with water and brine, dried over MgSO₄ and concentrated in vacuo. The concentrated product was purified by column chromatography on silica gel to obtain Int C (56 mg, yield 58.1%).

### Preparation Example 8: 4-(difluoro(3-isopropyl-4-(methoxymethoxy)phenyl) methyl)-3,5-dimethylphenol (Int D)

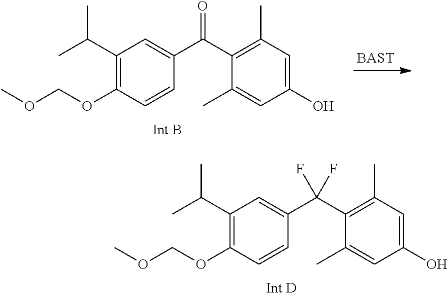 [SMILES: COCOc1ccc(C(=O)c2c(C)cc(O)cc2C)cc1C(C)C ; COCOc1ccc(C(F)(F)c2c(C)cc(O)cc2C)cc1C(C)C]

A mixture of Int B (0.1 g, 0.3 mmol) in 1,2-Dichlorethan (1 ml) was added BAST (0.2 g, 0.9 mmol) was stirred for 72 hours at RT under N₂. The reaction as quenched with saturated aqueous NaHCO₃(1 ml). Then the organic phase was separated and washed with water and brine, dried over MgSO₄ and concentrated in vacuo. The concentrated product was purified by column chromatography on silica gel to obtain Int D (66 mg, yield 61.9%).

### Preparation Example 9: 4-(4-(methoxymethoxy)-3-(propan-2-yl-1,1,1,3,3,3-d6)benzyl)-3,5-dimethylphenol (Int E)

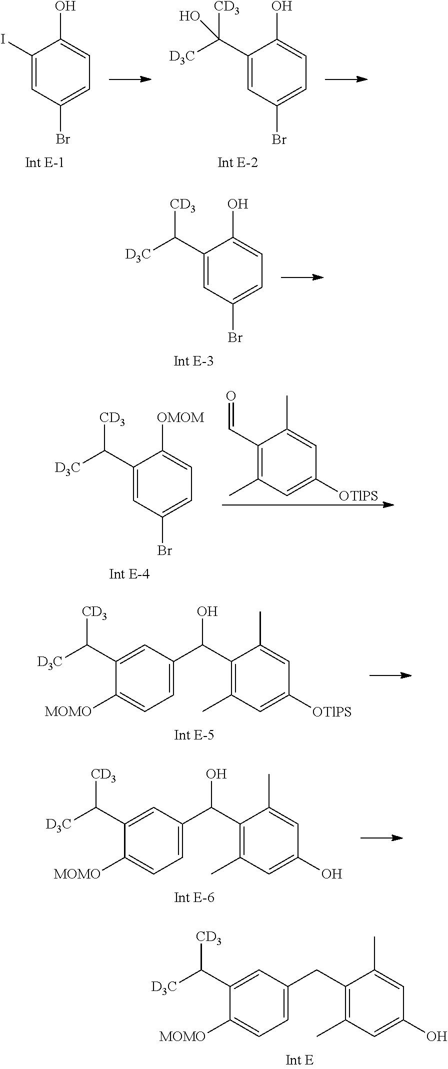

iPrMgCl (1 M in THF, 50 ml) was added dropwise to a solution of Int E-1 (5.0 g, 16.7 mmol) in THF (50 ml) at −20° C. and stirred for 2 hours. Acetone-D6 (0.5 ml) was added dropwise into the solution and stirred for 2 h, then naturally warm to room temperature. The mixture reaction was quenched with saturated NH₄Cl solution (30 ml) and EtOAc (50 ml) was added into the mixture reaction. Then the organic phase was separated and washed with water and brine, dried over MgSO₄ and concentrated in vacuo. The concentrated product was purified by column chromatography on silica gel to obtain Int E-2 (3.2 g, yield 80.1%).

Et3SiH (2.94 g, 25.3 mmol) was added to a solution of Int E-2 (3.0 g, 12.6 mmol) and TFA (0.1 ml) in DCE (30 ml) and stirred for 4 h. Water (30 ml) was added into the mixture reaction. Then the organic phase was separated and washed with water and brine, dried over MgSO₄ and concentrated in vacuo to obtain Int E-3 (2.6 g, yield 92.9%).

tBuOK (1.98 g, 17.6 mmol) was added to a solution of Int E-3 (2.6 g, 11.7 mmol) in THF (30 ml) at 0° C. and stirred for 1 h. Then the MOMBr (1.62 g, 12.9 mmol) was added and stirred for 2 h. The mixture reaction was quenched with saturated NH₄Cl solution (30 ml) and EtOAc (50 ml) was added into the mixture reaction. Then the organic phase was separated and washed with water and brine, dried over MgSO₄ and concentrated in vacuo. The concentrated product was purified by column chromatography on silica gel to obtain Int E-4 (1.9 g, yield 60.9%).

n-BuLi (4.3 ml, 2.5 M in hexane) was added to a solution of Int E-4 (1.9 g, 7.16 mmol) in THF (20 ml) at −78° C. and stirred for 30 min. Then 2,6-dimethyl-4-((triisopropylsilyl)oxy) benzaldehyde (2.31 g, 7.52 mmol) was added dropwise and stirred for 1 h at −78° C. The mixture reaction was quenched with saturated NH₄Cl solution (20 ml) and EtOAc (40 ml) was added into the mixture reaction. Then the organic phase was separated and washed with water and brine, dried over MgSO₄ and concentrated in vacuo. The concentrated product was purified by column chromatography on silica gel to obtain Int E-5 (2.43 g, yield 68.8%).

A solution of TBAF (5 ml, 1 M in EA) was added to a solution of Int E-5 (2.43 g, 4.93 mmol) in EtOAc (20 ml) and stirred for 30 min. Water (20 ml) was added and stirred for 15 min. Then the organic phase was separated and washed with water and brine, dried over MgSO₄ and concentrated in vacuo. The residue was crystalized by n-heptane to obtain Int E-6 (1.32 g, yield 79.6%).

Pd/C (0.1 g, 30 wt %) was added to a solution of Int E-6 (1.32 g, 3.92 mmol) and TFA (2 drops) in DCM (15 ml). The solution was stirred for 4 h under H₂ at RT. After filtration, the solution was concentrated in vacuo to obtain Int E (1.15 g, yield 91.5%).

### Preparation Example 10: phenyl hydrogen ((4-(4-hydroxy-3-isopropylbenzyl)-3,5-dimethylphenoxy)methyl)phosphonate (Int F)

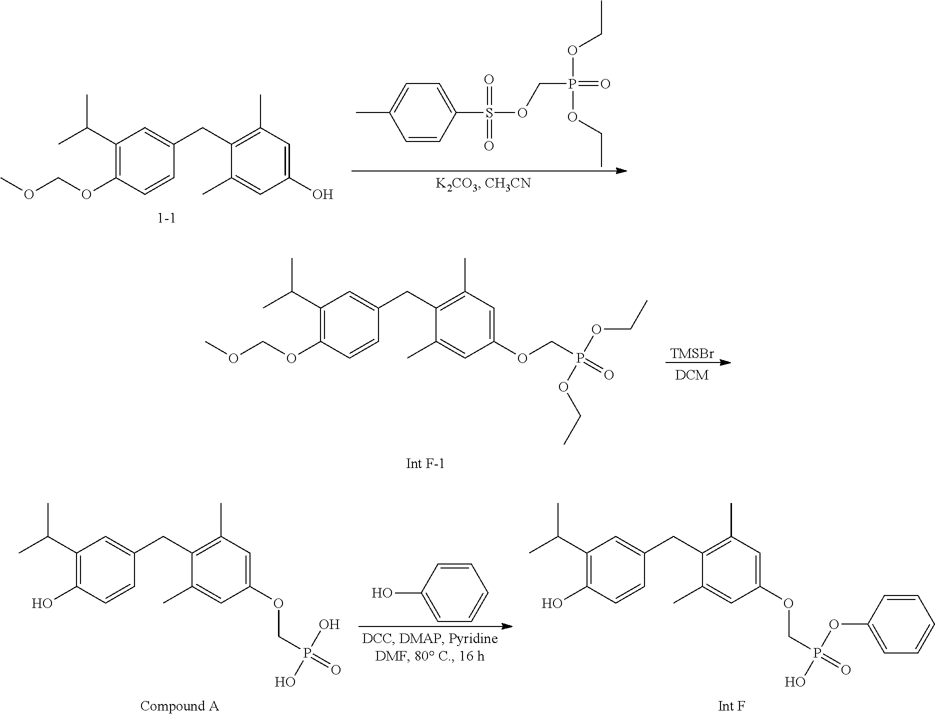

Potassium carbonate (3.30 g, 23.85 mmol) was added to a solution of 4-[(4-(methoxymethoxy)-3-(propan-2-yl)phenyl)methyl]-3,5-dimethylphenol (5 g, 15.90 mmol), and diethyl [(4-methylbenzenesulfonyl)oxy] methanephosphonate (5.12 g, 15.9 mmol) in acetonitrile (30 ml), and stirred for 4 h at 80-85° C. After cooling, water and ethyl acetate were added to the mixture. The organic layer was separated and concentrated under reduced pressure to provide diethyl [(4-[(4-(methoxymethoxy)-3-(propan-2-yl)phenyl)methyl]-3,5-dimethylphenoxy)methyl]phosphonate (7.2 g, yield 97.47%) as an oil.

TMSBr (3.30 g, 21.6 mmol) was add dropwise to a solution of diethyl [(4-[(4-(methoxymethoxy)-3-(propan-2-yl)phenyl)methyl]-3,5-dimethylphenoxy)methyl]phosphonate (5 g, 10.76 mmol) in dichloromethane (25 ml) and stirred for 4 h at 10-15° C. Water was added dropwise to the mixture. The organic layer was separated and concentrated under reduced pressure to obtain compound A (3.6 g, yield 92%).

Pyridine (200 ml), DCC (92 g, 451 mmol) and DMAP (18 g, 147 mmol) were added to a solution of compound A (55 g, 151 mmol) and phenol (28.4 g, 302 mmol) in DMF (1 L) and stirred overnight at 80-85° C. After cooling, ethyl acetate was added to the mixture and adjusted to the pH 3.0 by the 1N HCl solution. The organic layer was separated, washed with water, dried over (Na₂SO₄) and evaporated to dryness to afford crude product. Then the crude product was purified by silica gel chromatography eluted with PE:EtOAc=10:1 to obtain Int F (26 g, yield: 39%) as a white solid.

### Preparation Example 11 Pentan-3-yl ((R)-((4-(4-hydroxy-3-isopropylbenzyl)-3,5-dimethylphenoxy)methyl)(phenoxy)phosphoryl)-L-alaninate (29) and Pentan-3-yl ((S)-((4-(4-hydroxy-3-isopropylbenzyl)-3,5-dimethylphenoxy)methyl) (phenoxy)phosphoryl)-L-alaninate (30)

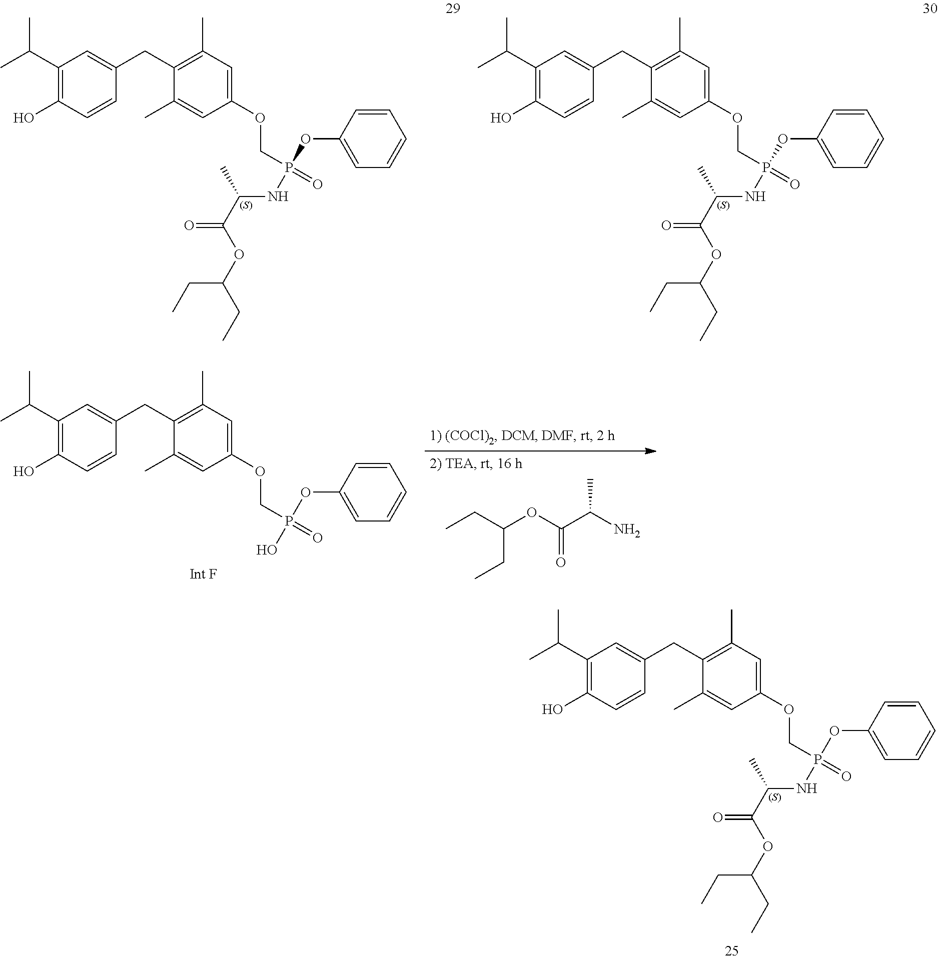

Oxalyl chloride (8.6 g, 67.7 mmol) was added dropwise to a solution of Int F (10 g, 22.7 mmol) and DMF (166 mg, 2.27 mmol) in DCM (100 ml) and stirred for 2 h at RT. The reaction mixture was concentrated to dryness to afford an oil. The oil was dissolved into DCM (100 ml), followed by addition of pentan-3-yl L-alaninate (18.05 g, 113.5 mmol) and stirred for 2 h at RT. The reaction mixture was concentrated, purified by silica gel chromatography, and eluted with PE:EtOAc=10:1 to obtain compound 25 (3.0 g, yield) The compound 25 was purified by chiral column (Welch XT C18 150 mm*21.2 mm, 5 um) to afford compound 28, (1.5 g, yield) and compound 29 (1.2 g, yield).

### Preparation Example 12 pentan-3-yl (((4-(3-(4-fluorobenzyl)-4-hydroxybenzyl)-3,5-dimethylphenoxy)methyl)(phenoxy)phosphoryl)-L-alaninate(52)

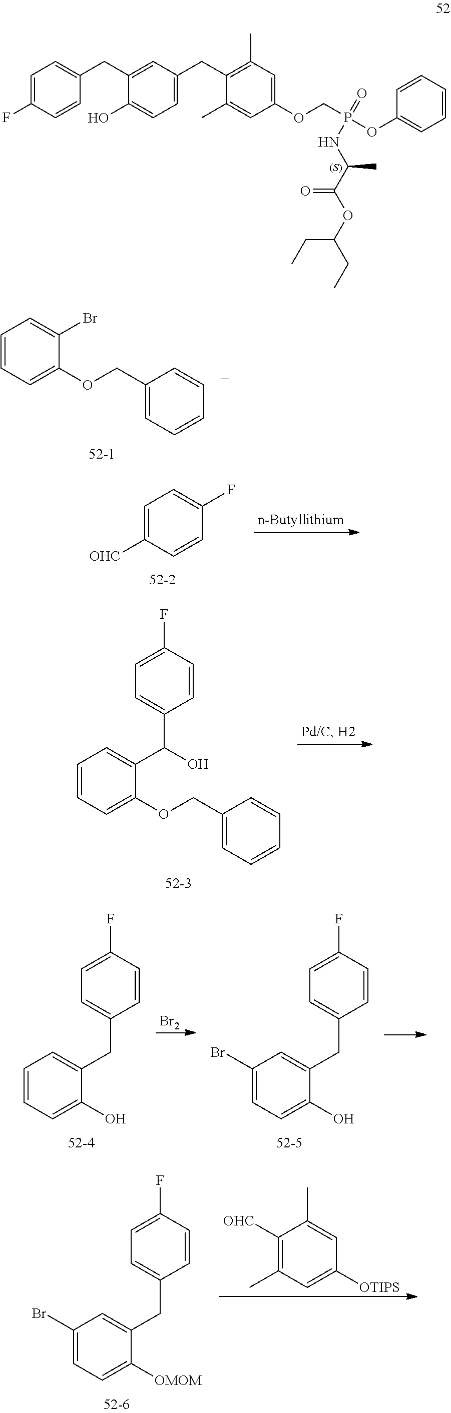

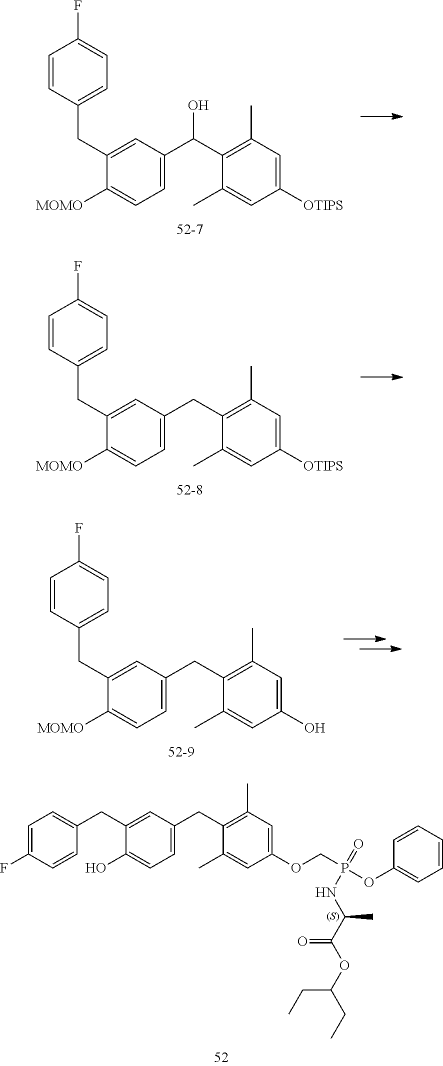

To a stirred solution of 1-(benzyloxy)-2-bromobenzene (21 g, 79.81 mmol) in THF (S0, 100 mL) were added butyllithium (5.62 g, 87.79 mmol) dropwise at −70° C. under nitrogen atmosphere. The resulting mixture was stirred for 0.5 h at −70° C. under nitrogen atmosphere. To the above mixture was added 4-fluorobenzaldehyde (R1, 9.91 g, 79.81 mmol, Purity 100%) dropwise over 15 min at −70° C. The resulting mixture was stirred for additional 1 h at 0° C. The reaction was quenched by the addition of [water] (50 mL) at 25° C. The resulting mixture was extracted with EA (3×100 mL). The combined organic layers were washed with [brine](2×50 mL), dried over anhydrous Na₂SO₄. After filtration, the filtrate was concentrated under reduced pressure.

A solution of (2-(benzyloxy)phenyl)(4-fluorophenyl)methanol (27 g, 87.56 mmol), hydrogen chloride (0.32 g, 8.76 mmol) and Pd/C (4.92 g, 35.02 mmol) in methanol (60 mL) was stirred for 16 h at 25° C. under H₂ atmosphere. The resulting mixture was filtered, the filter cake was washed with MeOH (3×20 mL). The filtrate was neutralized to pH 7-8 with NaHCO₃(aq.). The resulting mixture was concentrated under reduced pressure. The aqueous layer was extracted with DCM (3×50 mL). The combined organic layers were dried over anhydrous Na₂SO₄. After filtration, the filtrate was concentrated under reduced pressure. The crude product was used in the next step directly without further purification.

To a stirred solution of 2-[(4-fluorophenyl)methyl]phenol (15 g, 74.18 mmol) in DCM (50 mL) were added Tetrabutylammonium tribromide (37.56 g, 77.89 mmol) in portions at 0° C. The resulting mixture was stirred for 3 h at 25° C. The resulting mixture was concentrated under reduced pressure. The reaction was quenched with water (50 mL) at 25° C. The resulting mixture was extracted with EA (2×100 mL). The combined organic layers were washed with brine (2×20 mL), dried over anhydrous Na₂SO₄. After filtration, the filtrate was concentrated under reduced pressure. The residue was purified by silica gel column chromatography, eluted with EA:PE (1:10) to afford 4-bromo-2-[(4-fluorophenyl)methyl]phenol (12 g, Yield 57.55%) as a yellow oil.

To a stirred solution of 4-bromo-2-[(4-fluorophenyl)methyl]phenol (12 g, 42.69 mmol) in THF (50 mL) were added Sodium hydride (2.22 g, 55.50 mmol, Purity 60%) in portions at 0° C. The resulting mixture was stirred for 0.5 h at 25° C. To the above mixture was added chloro(methoxy)methane (4.12 g, 51.23 mmol) dropwise over 15 min at 0° C. The resulting mixture was stirred for additional 1 h at 25° C. The reaction was quenched by the addition of water (30 mL) at 25° C. The resulting mixture was extracted with EA (3×30 mL). The combined organic layers were washed with brine (2×20 mL), dried over anhydrous Na₂SO₄. After filtration, the filtrate was concentrated under reduced pressure. The residue was purified by silica gel column chromatography, eluted with [EA:PE (1:10)] to afford 4-bromo-2-[(4-fluorophenyl)methyl]-1-(methoxymethoxy)benzene (10 g, Yield 72.04%) as a yellow oil.

To a stirred solution of 4-bromo-2-[(4-fluorophenyl)methyl]-1-(methoxymethoxy)benzene (15 g, 46.13 mmol) in THF (50 mL) was added butyllithium (3.10 g, 48.44 mmol) dropwise at −70° C. under nitrogen atmosphere. The resulting mixture was stirred for 0.5 h at −70° C. under nitrogen atmosphere. To the above mixture was added 2,6-dimethyl-4-[(tris(propan-2-yl)silyl)oxy]benzaldehyde (14.14 g, 46.13 mmol) dropwise over 15 min at −70° C. The resulting mixture was stirred for additional 1 h at 0° C. The reaction was quenched by the addition of water (30 mL) at 25° C. The resulting mixture was extracted with EA (2×50 mL). The combined organic layers were washed with brine (2×20 mL), dried over anhydrous Na₂SO₄. After filtration, the filtrate was concentrated under reduced pressure. The residue was purified by silica gel column chromatography, eluted with EA:PE (0-1:10) to afford (2,6-dimethyl-4-[(tris(propan-2-yl)silyl)oxy]phenyl)(3-[(4-fluorophenyl)methyl]-4-(methoxymethoxy)phenyl)methanol (10 g, Yield 39.22%) as a yellow oil.

To a stirred solution of 4-[(3-[(4-fluorophenyl)methyl]-4-(methoxymethoxy)phenyl)(hydroxy)methyl]-3,5-dimethylphenol (3.5 g, 9.20 mmol) and Pd/C (0.26 g, 1.84 mmol) in methanol (40 mL) were added hydrogen chloride (0.050 g, 1.38 mmol) dropwise at 25° C. The resulting mixture was stirred for 16 h at 25° C. under H₂ atmosphere. The resulting mixture was filtered, the filter cake was washed with MeOH (3×10 mL). The filtrate was The residue was basified to pH 7-8 with NaHCO₃(aq.). The resulting mixture was filtered, the filter cake was washed with DCM (3×10 mL). The filtrate was extracted with DCM (3×50 mL). The combined organic layers were dried over anhydrous Na₂SO₄. After filtration, the filtrate was concentrated under reduced pressure. This resulted in 4-[(3-[(4-fluorophenyl)methyl]-4-(methoxymethoxy)phenyl)methyl]-3,5-dimethylphenol (2 g, Yield 54.84%) as a yellow oil.

To a stirred solution of (2,6-dimethyl-4-[(tris(propan-2-yl)silyl)oxy]phenyl)(3-[(4-fluorophenyl)methyl]-4-(methoxymethoxy)phenyl)methanol (10 g, 18.09 mmol) in THF (50 mL) were added tetrabutylammonium fluoride (4.73 g, 18.09 mmol) dropwise at 0° C. The resulting mixture was stirred for 1 h at 25° C. The resulting mixture was concentrated under reduced pressure. The residue was dissolved in EA (100 mL). The organic layers were washed with water (2×30 mL), dried over anhydrous Na₂SO₄. After filtration, the filtrate was concentrated under reduced pressure. The residue was purified by trituration with Heptane (20 mL). The precipitated solid was collected by filtration and washed with Heptane (3×5 mL). This resulted in 4-[(3-[(4-fluorophenyl)methyl]-4-(methoxymethoxy)phenyl)(hydroxy)methyl]-3,5-dimethylphenol (5.5 g, Yield 76.69%) as a yellow solid.

The following Synthesis method can follow the Preparation example 10 and example 11, and the compound 52 was obtained.

### Preparation Example 13 pentan-3-yl ((4-(3-(sec-butyl)-4-hydroxybenzyl)-3,5-dimethylphenethyl)(phenoxy)phosphoryl)-L-alaninate(53)

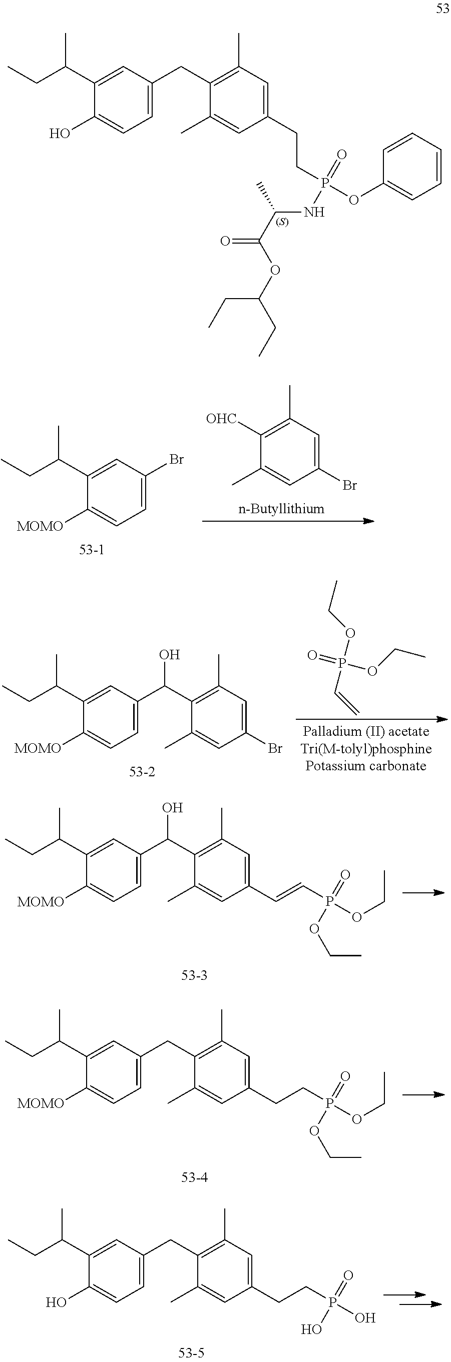

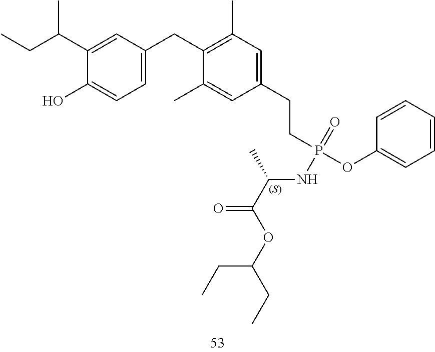

To a solution of 4-bromo-2-(butan-2-yl)-1-(methoxymethoxy)benzene (6.11 g, 22.37 mmol) in THF (40 mL) was added butyllithium (1.58 g, 24.61 mmol) drop-wise at −70° C., then the reaction mixture was stirred for 1 h. Then a solution of 4-bromo-2,6-dimethylbenzaldehyde (4.77 g, 22.37 mmol) in THF (20 mL) was added to above solution at −70° C. under N₂. The reaction mixture was stirred at −70° C. for 0.5 hours. After warming to room temperature, NH₄Cl (aq.) was added and stirred 30 mins. And extract with ethyl acetate. The reaction mixture was concentrated, purified by silica gel chromatography, and eluted with PE:EtOAc=20:1 to obtain compound 53-2, 5.23 g, yield: 57.4%.

53-2 (3.78 g, 9.28 mmol), diethyl ethenylphosphonate (1.68 g, 10.21 mmol), Palladium(II) acetate (0.21 g, 0.93 mmol), Potassium carbonate (1.54 g, 11.14 mmol) and Tri(m-tolyl)phosphine (0.28 g, 0.93 mmol) in DMF (20 mL) was de-gassed and then heated to 110° C. for 16 hours under N₂. The reaction was cool to rt and H₂O was added to the above solution. The mixture was extracted with EA (100 mL*3). The combined organic phase was washed with brine, filtered and concentrated in vacuo. The residue was purified by silica gel chromatography (PE:EA=5:1-1:2) to give the pure product as yellow oil, 53-3, 2.78 g, yield: 61.1%.

53-3 (40 mg, 0.082 mmol), Pd/C (0.00087 g, 0.0082 mmol), trifluoroacetic acid (0.00047 g, 0.0041 mmol) in dichloromethane (2 mL). The reaction was filtered and concentrated in vacuo. The residue was purified by TLC (PE:EA=1:1) to give the pure product as white oil, 53-4, 21 mg, yield: 54.04%.

The following Synthesis method can follow the Preparation example 10 and example 11, and the compound 53 was obtained.

The following compounds (in Table 1) were prepared according to the procedures described herein using the appropriate starting material(s) and appropriate protecting group chemistry as needed, and characterized by ¹HNMR as follows:

TABLE 1
Compound list.

| No. | Structure | ¹HNMR(400M, d₆-DMSO) |
|---|---|---|
| 1 | 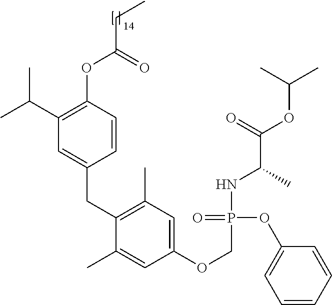 | δ 7.44-7.35 (m, 2H), 7.23-7.08 (m, 5H), 7.03-7.00 (m, 1H), 6.61 (s, 2H), 5.17(s, 1H), 5.06-4.98 (m, 1H), 4.43-4.30 (m, 2H), 3.91- 3.82 (m, 3H), 3.22-3.12 (m, 1H), 2.53 (t, J = 8.4 Hz, 2H), 2.26 (s, 6H), 1.68-1.56 (m, 2H), 1.35 (d, J = 7.2 Hz, 3H), 1.35-1.12 (m, 36H), 0.93-0.85 (m, 3H). |
| 2 | 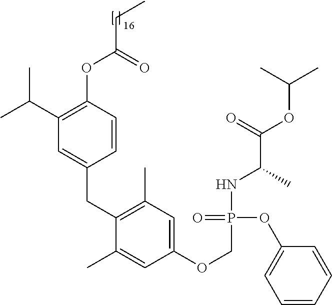 | δ 7.44-7.35 (m, 2H), 7.23-7.08 (m, 5H), 7.03-7.00 (m, 1H), 6.61 (s, 2H), 5.17 (s, 1H), 5.06-4.98 (m, 1H),4.41-4.32 (m, 2H), 3.91- 3.82 (m, 3H), 3.22-3.12 (m, 1H), 2.53 (t, J = 8.4 Hz, 2H), 2.26 (s, 6H), 1.70-1.55 (m, 2H), 1.35 (d, J = 7.2 Hz, 3H), 1.34-1.12 (m, 40H), 0.93-0.85 (m, 3H). |
| 3 | 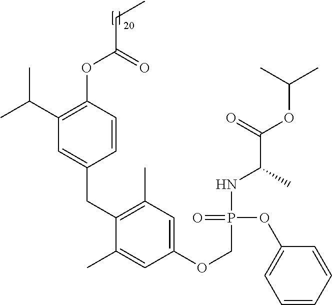 | δ 7.44-7.35 (m, 2H), 7.23-7.08 (m, 5H), 7.03-7.00 (m, 1H), 6.61 (s, 2H), 5.17(s, 1H), 5.06-4.98 (m, 1H), 4.41-4.32 (m, 2H), 3.91- 3.82 (m, 3H), 3.22-3.12 (m, 1H), 2.53 (t, J = 8.4 Hz, 2H), 2.26 (s, 6H), 1.70-1.55 (m, 2H), 1.35 (d, J = 7.2 Hz, 3H), 1.34-1.12 (m, 48H), 0.93-0.85 (m, 3H). |
| 4 | 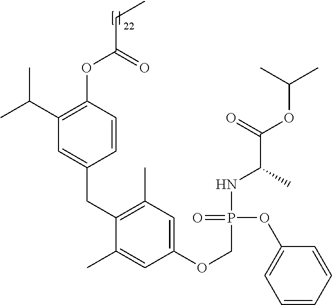 | δ 7.44-7.35 (m, 2H), 7.23-7.08 (m, 5H), 7.03-7.00 (m, 1H), 6.61 (s, 2H), 5.17 (s, 1H), 5.06-4.98 (m, 1H), 4.41-4.32 (m, 2H), 3.91- 3.82 (m, 3H), 3.22-3.12 (m, 1H), 2.53 (t, J = 8.4 Hz, 2H), 2.26 (s, 6H), 1.70-1.55 (m, 2H), 1.35 (d, J = 7.2 Hz, 3H), 1.34-1.12 (m, 52H), 0.93-0.85 (m, 3H). |
| 5 | 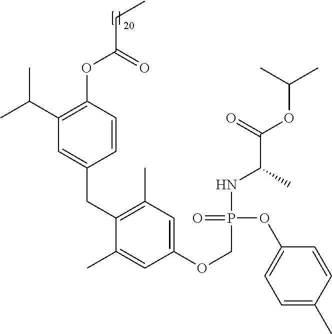 | δ 7.23-7.15 (m, 3H), 7.14-7.08 (m, 3H), 7.04-7.00 (m, 1H), 6.61 (s, 2H), 5.17 (d, J = 9.2 Hz, 1H), 5.06-4.94 (m, 1H), 4.41-4.32 (m, 2H), 3.92-3.80 (m, 3H), 3.22- 3.12 (m, 1H), 2.53 (t, J = 8.4 Hz, 2H), 2.34 (s, 3H), 2.26 (s, 6H), 1.68-1.56 (m, 2H), 1.35 (d, J = 7.2 Hz, 3H), 1.34-1.15 (m, 48H), 0.94-0.85 (m, 3H). |
| 6 | 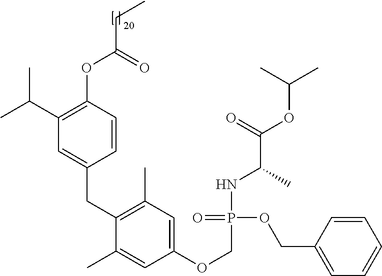 | δ 7.39-7.23 (m, 5H), 7.22-7.17 (m, 1H), 7.13-7.08 (m, 1H), 7.03- 6.95 (m, 1H), 6.61 (s, 2H), 5.05- 4.95 (m, 1H), 4.80-4.69 (m, 3H), 4.28 (dd, J = 11.6, 0.8 Hz, 2H), 3.90-3.81 (m, 3H), 3.25-3.10 (m, 1H), 2.53 (t, J = 8.4 Hz, 2H), 2.26 (s, 6H), 1.70-1.55 (m, 2H), 1.36 (d, J = 7.2 Hz, 3H), 1.30- 1.15 (m, 48H), 0.93-0.85 (m, 3H). |
| 7 | 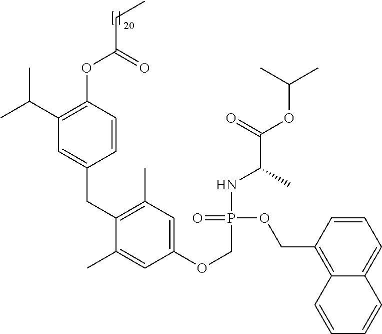 | δ 8.05-8.00 (m, 1H),7.93-7.84 (m, 2H), 7.57-7.38 (m, 4H), 7.22- 7.15 (m, 1H), 7.15-7.08 (m, 1H), 7.05-7.00 (m, 1H), 6.61 (s, 2H), 5.21-4.96 (m, 3H), 4.74 (s, 1H), 4.28 (dd, J = 12.0, 0.8 Hz, 2H), 3.90-3.81 (m, 3H), 3.25- 3.10 (m, 1H), 2.53 (t, J = 8.4 Hz, 2H), 2.26 (s, 6H), 1.68-1.57 (m, 2H), 1.36 (d, J = 7.2 Hz, 3H), 1.34-1.12 (m, 48H), 0.93-0.85 (m, 3H). |
| 8 | 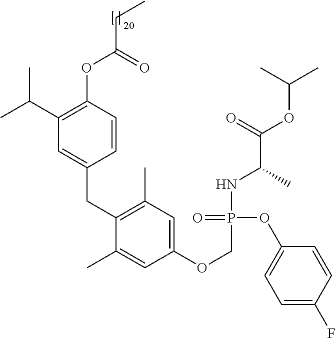 | δ 7.23-7.07 (m, 6H), 7.04-7.00 (m, 1H), 6.61 (s, 2H), 5.17 (d, J = 9.2 Hz, 1H), 5.06-4.96 (m, 1H), 4.41-4.32 (m, 2H), 3.91-3.82 (m, 3H), 3.22-3.12 (m, 1H), 2.53 (t, J = 8.4 Hz, 2H), 2.26 (s, 6H), 1.68-1.56 (m, 2H), 1.35 (s, 3H), 1.38-1.12 (m, 48H), 0.93-0.85 (m, 3H). |
| 9 | 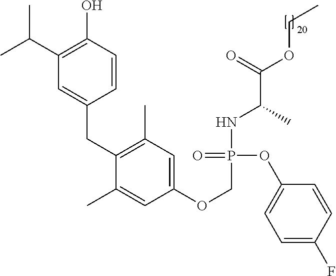 | δ 9.10 (s, 1H), 7.23-7.07 (m, 4H), 7.02-6.92 (m, 2H), 6.77-6.72 (m, 1H), 6.61 (s, 2H), 5.13 (s, 1H), 4.43-4.30 (m, 2H), 4.09 (t, J = 6.4 Hz, 2H), 3.95-3.83 (m, 3H), 3.17- 3.07 (m, 1H), 2.26 (s, 6H), 1.72- 1.60 (m, 2H), 1.36 (s, 3H), 1.30- 1.12 (m, 42H), 0.93-0.85 (m, 3H). |
| 10 | 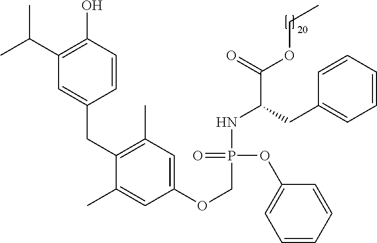 | δ 9.10 (s, 1H), 7.44-7.35 (m, 2H), 7.30-7.17 (m, 5H), 7.15-7.10 (m, 3H), 7.02-6.95 (m, 2H), 6.74 (d, J = 8.8 Hz, 1H), 6.61 (s, 2H), 5.09 (d, J = 10.0 Hz, 1H), 4.46- 4.30 (m, 2H), 4.10-3.95 (m, 3H), 3.87-3.82 (m, 2H), 3.17-3.07 (m, 1H), 3.01-2.91 (m, 2H), 2.26 (s, 6H), 1.72-1.60 (m, 2H), 1.43- 1.15 (m, 42H) , 0.95-0.85 (m, 3H). |
| 11 | 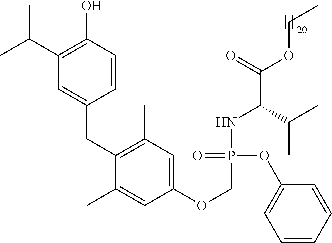 | δ 9.10 (s, 1H), 7.44-7.35 (m, 2H), 7.20-7.10 (m, 3H), 7.02-6.92 (m, 2H), 6.74 (d, J = 8.8 Hz, 1H), 6.61 (s, 2H), 5.00 (d, J = 10.4 Hz, 1H), 4.47-4.30 (m, 2H), 4.15- 4.04 (m, 2H), 3.87-3.82 (m, 2H), 3.70-3.62 (m, 1H), 3.17-3.07 (m, 1H), 2.26 (s, 6H), 2.10-1.97 (m, 1H), 1.72-1.60 (m, 2H), 1.43- 1.14 (m, 42H), 0.93 (d, J = 0.6 Hz, 3H), 0.94-0.85 (m, 6H). |
| 12 | 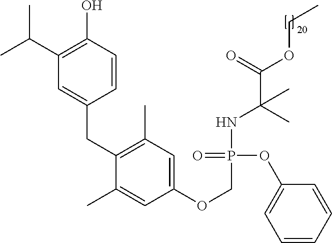 | δ 9.10 (s, 1H), 7.44-7.35 (m, 2H), 7.20-7.10 (m, 3H), 7.02-6.92 (m, 2H), 6.77-6.72 (m, 1H), 6.61 (s, 2H), 4.90 (s, 1H), 4.51-4.34 (m, 2H), 4.08-4.02 (m, 2H) , 3.88- 3.82 (m, 2H), 3.17-3.07 (m, 1H), 2.26 (s, 6H), 1.72-1.61 (m, 2H), 1.50 (s, 6H), 1.43-1.15 (m, 42H), 0.93-0.85 (m, 3H). |
| 13 | 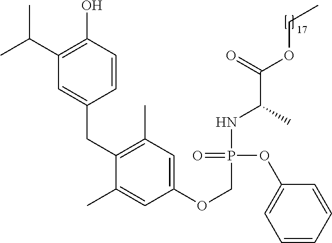 | δ 9.10 (s, 1H), 7.44-7.35 (m, 2H), 7.20-7.10 (m, 3H), 7.00-6.90 (m, 2H), 6.74 (d, J = 8.8 Hz, 1H), 6.61 (s, 2H), 5.12 (s, 1H), 4.43-4.30 (m, 2H), 4.14-4.06 (m, 2H), 3.95- 3.83 (m, 3H), 3.18-3.10 (m, 1H), 2.26 (s, 6H), 1.72-1.60 (m, 2H), 1.43-1.14 (m, 39H), 0.93-0.85 (m, 3H). |
| 14 | 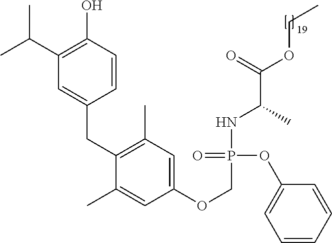 | δ 9.10 (s, 1H), 7.44-7.35 (m, 2H), 7.20-7.10 (m, 3H), 7.00-6.90 (m, 2H), 6.74 (d, J = 8.8 Hz, 1H), 6.61 (s, 2H), 5.12 (s, 1H), 4.43- 4.30 (m, 2H), 4.14-4.06 (m, 2H), 3.95-3.83 (m, 3H), 3.18-3.10 (m, 1H), 2.26 (s, 6H), 1.72-1.60 (m, 2H), 1.43-1.14 (m, 43H), 0.93- 0.85 (m, 3H). |
| 15 | 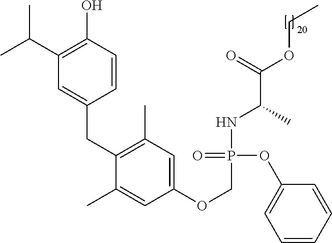 | δ 9.10 (s, 1H), 7.44-7.35 (m, 2H), 7.20-7.10 (m, 3H), 7.00-6.90 (m, 2H), 6.74 (d, J = 8.8 Hz, 1H), 6.61 (s, 2H), 5.12 (s, 1H), (4.43- 4.30 (m, 2H), 4.14-4.06 (m, 2H), 3.95-3.83 (m, 3H),3.18-3.10 (m, 1H), 2.26 (s, 6H), 1.72-1.60 (m, 2H), 1.43-1.14 (m, 45H), 0.93- 0.85 (m, 3H). |
| 16 | 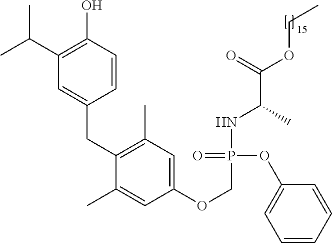 | δ 9.10 (s, 1H), 7.44-7.35 (m, 2H), 7.20-7.10 (m, 3H), 7.00-6.90 (m, 2H), 6.74 (d, J = 8.8 Hz, 1H), 6.61 (s, 2H), 5.12 (s, 1H), 4.43- 4.30 (m, 2H), 4.14-4.06 (m, 2H), 3.95-3.83 (m, 3H), 3.18-3.10 (m, 1H), 2.26 (s, 6H), 1.72-1.60 (m, 2H), 1.43-1.14 (m, 35H), 0.93- 0.85 (m, 3H). |
| 17 | 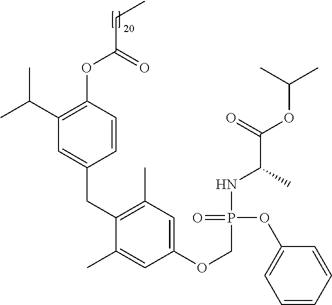 | δ 7.44-7.35 (m, 2H), 7.23-7.08 (m, 5H), 7.03-7.01 (m, 1H), 6.61 (s, 2H), 5.17 (s, 1H), 5.04- 4.98 (m, 1H), 4.41-4.32 (m, 2H), 3.91-3.82 (m, 3H), 3.22-3.13 (m, 1H), 2.56-2.48 (m, 2H), 2.26 (s, 6H), 1.68-1.56 (m, 2H), 1.35 (d, J = 7.2 Hz, 3H), 1.32-1.12 (m, 48H), 0.93-0.85 (m, 3H). |
| 18 | 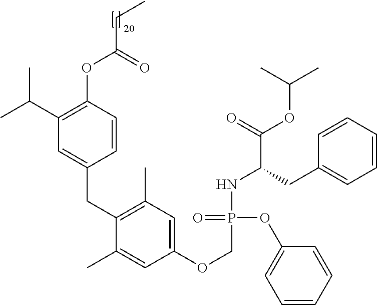 | δ 7.44-7.35 (m, 2H), 7.30-7.08 (m, 10H), 7.04-7.00 (m, 1H), 6.61 (s, 2H), 5.08-4.94 (m, 2H), 4.46-4.30 (m, 2H), 4.00-3.93 (m, 1H), 3.90-3.85 (m, 2H), 3.22- 3.12 (m, 1H), 3.05-2.88 (m, 2H), 2.57-2.48 (m, 2H), 2.26 (s, 6H), 1.68-1.55 (m, 2H) 1.34- 1.12 (m, 48H), 0.93-0.85 (m, 3H). |
| 19 | 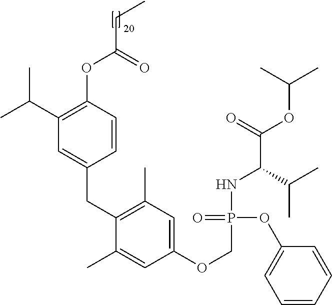 | δ 7.44-7.35 (m, 2H), 7.23-7.08 (m, 5H), 7.03-7.01 (m, 1H), 6.61 (s, 2H), 5.13-5.01 (m, 2H), 4.47- 4.30 (m, 2H), 3.86 (s, 2H), 3.68- 3.62 (m, 1H), 3.21-3.15 (m, 1H), 2.53 (t, J = 8.4 Hz, 2H), 2.26 (s, 6H), 1.90-1.80 (m, 1H), 1.70- 1.55 (m, 2H), 1.34-1.12 (m, 48H), 0.95-0.85 (m, 9H). |
| 20 | 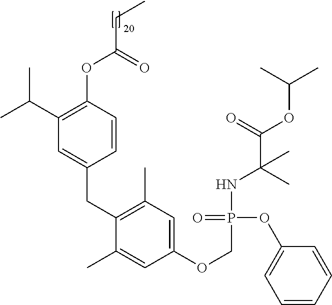 | δ 7.44-7.35 (m, 2H), 7.23-7.14 (m, 5H), 7.04-7.01 (m, 1H), 6.61 (s, 2H), 5.01-4.95 (m, 1H), 4.86 (s, 1H), 4.51-4.34 (m, 2H), 3.88- 3.82 (m, 2H), 3.22-3.12 (m, 1H), 2.53 (t, J = 8.4 Hz, 2H), 2.26 (s, 6H), 1.69-1.56 (m, 2H), 1.51 (s, 6H), 1.34-1.22 (m, 48H), 0.93- 0.85 (m, 3H). |
| 21 | 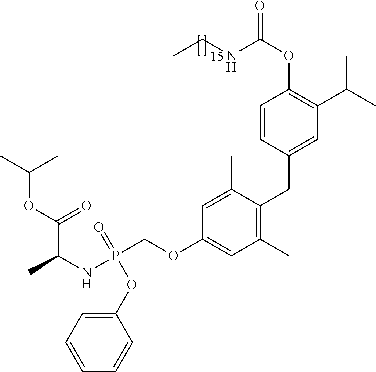 | δ 7.44-7.35 (m, 2H), 7.23-7.15 (m, 3H), 7.15-7.10 (m, 2H), 7.05- 7.00 (m, 1H), 6.87-6.82 (m, 1H), 6.61 (s, 2H), 5.16 (s, 1H), 5.06-4.98 (m, 1H),4.43-4.30 (m, 2H), 3.91-3.82 (m, 3H), 3.25- 3.15 (m, 1H), 3.12-3.05 (m, 2H), 2.26 (s, 6H), 1.55-1.44 (m, 2H), 1.35 (d, J = 7.2 Hz, 3H), 1.35- 1.12 (m, 38H), 0.93-0.85 (m, 3H). |
| 22 | 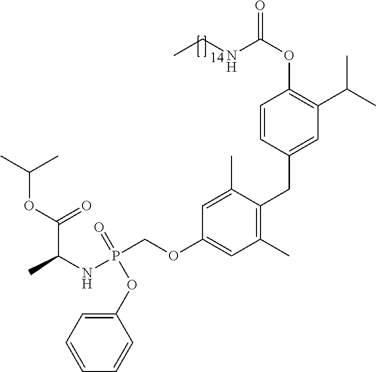 | δ 7.44-7.35 (m, 2H), 7.21-7.09 (m, 4H), 7.02-6.95 (m, 1H), 6.88 (d, J = 8.4 Hz, 1H), 6.61 (s, 2H), 5.70 (s, 2H), 5.17 (s, 1H), 5.06- 4.95 (m, 1H), 4.41-4.32 (m, 2H), 3.91-3.82 (m, 3H), 3.26-3.16 (m, 1H), 2.45-2.35 (m, 2H), 2.26 (s, 6H), 1.62-1.50 (m, 2H), 1.35 (d, J = 7.2 Hz, 3H), 1.34-1.15 (m, 36H), 0.93-0.85 (m, 3H). |
| 23 | 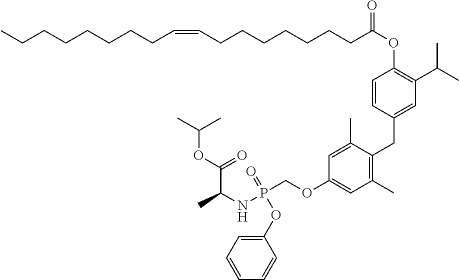 [SMILES: CCCCCCCC/C=C\CCCCCCCC(=O)Oc1ccc(Cc2c(C)cc(OCP(=O)(N[C@@H](C)C(=O)OC(C)C)Oc3ccccc3)cc2C)cc1C(C)C] | δ 7.44-7.35 (m, 2H), 7.23-7.15 (m, 3H), 7.15-7.10 (m, 2H), 7.05- 7.00 (m, 1H), 6.61 (s, 2H), 5.37- 5.30 (m, 2H), 5.16 (s, 1H), 5.06- 4.98 (m, 1H), 4.43-4.30 (m, 2H), 3.91-3.82 (m, 3H), 3.20-3.10 (m, 1H), 2.60-2.50 (m, 2H), 2.26 (s, 6H), 2.08-1.94 (m, 4H), 1.69-1.56 (m, 2H), 1.44-1.15 (m, 35H), 0.95-0.85 (m, 3H). |
| 24 | 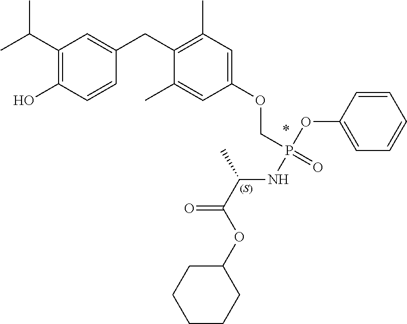 | δ 9.10 (s, 1H), 7.44-7.35 (m, 2H), 7.20-7.10 (m, 3H), 7.02-6.92 (m, 2H), 6.74 (d, J = 8.8 Hz, 1H), 6.61 (s, 2H), 5.17 (d, J = 9.4 Hz, 1H), 4.80-4.71 (m, 1H), 4.43- 4.30 (m, 2H), 3.97-3.80 (m, 3H), 3.20-3.05 (m, 1H), 2.26 (s, 6H), 1.92-1.78 (m, 2H), 1.75-1.60 (m, 4H), 1.59-1.37 (m, 4H), 1.34 (d, J = 7.2 Hz, 3H), 1.22 (d, J = 6.9 Hz, 3H), 1.17 (d, J = 6.9 Hz, 3H). |
| 25 | 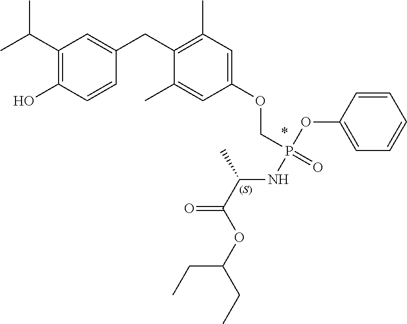 | δ 9.10 (s, 1H), 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80-4.70 (m, 1H), 4.43- 4.30 (m, 2H), 3.97-3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 26 | 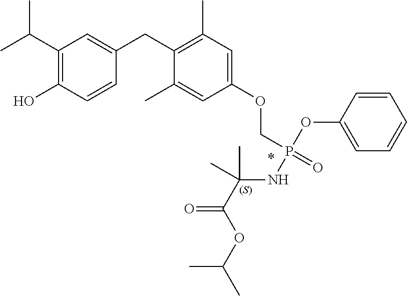 | δ 9.10 (s, 1H), 7.44-7.35 (m, 2H), 7.20-7.10 (m, 3H), 7.00-6.94 (m, 2H), 6.74 (d, J = 8.8 Hz, 1H), 6.61 (s, 2H), 5.05-4.95 (m, 1H), 4.87 (s, 1H), 4.51-4.34 (m, 2H), 3.86 (t, J = 1.2 Hz, 2H), 3.17- 3.07 (m, 1H), 2.26 (s, 6H), 1.51 (s, 6H), 1.28-1.17 (m, 12H). |
| 27 | 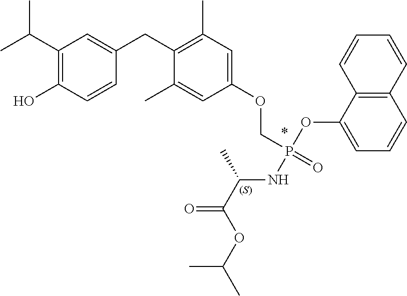 | δ 9.10 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 5.35 (d, J = 9.2 Hz, 1H), 5.05-4.95 (m, 1H), 4.38 (d, J = 2.8 Hz, 2H), 3.92-3.79 (m, 3H), 3.17-3.07 (m, 1H), 2.26 (s, 6H), 1.35 (d, J = 7.2 Hz, 3H), 1.28 (d, J = 5.7 Hz, 3H), 1.23 (dd, J = 6.4, 4.0 Hz, 6H), 1.17 (d, J = 6.8 Hz, 3H). |
| 28 | 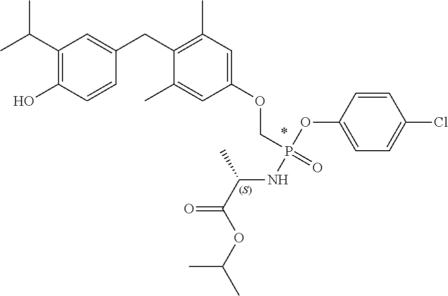 | δ 9.10 (s, 1H), 7.43-7.35 (m, 2H), 7.16-7.09 (m, 2H), 7.02-6.94 (m, 2H), 6.74 (d, J = 8.8 Hz, 1H), 6.61 (s, 2H), 5.17 (d, J = 9.4 Hz, 1H), 5.05-4.95 (m, 1H), 4.43- 4.30 (m, 2H), 3.92-3.80 (m, 3H), 3.17-3.07 (m, 1H), 2.26 (s, 6H), 1.35 (d, J = 7.2 Hz, 3H), 1.28 (d, J = 5.6 Hz, 3H), 1.23 (dd, J = 6.4, 4.0 Hz, 6H), 1.17 (d, J = 6.8 Hz, 3H). |
| 29 | 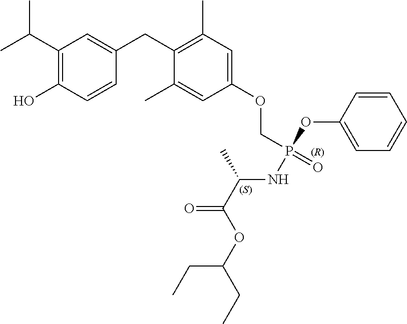 | δ 9.10 (s, 1H), 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80-4.74 (m, 1H), 4.43- 4.35 (m, 2H), 3.97-3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 30 | 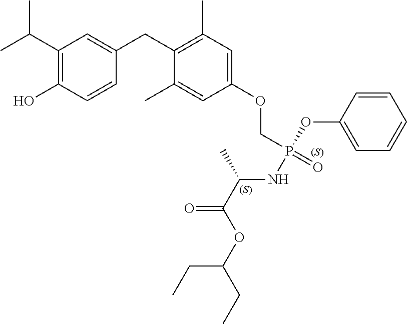 | δ 9.10 (s, 1H), 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80-4.72 (m, 1H), 4.43- 4.32 (m, 2H), 3.97-3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 31 | 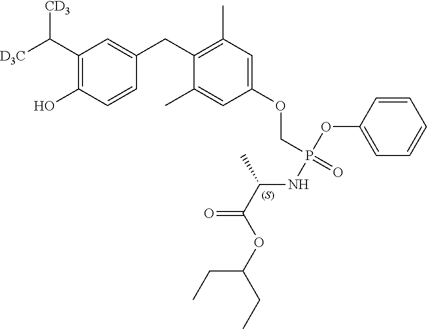 | δ 9.10 (s, 1H), 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80-4.70 (m, 1H), 4.43- 4.30 (m, 2H), 3.97-3.83 (m, 3H), 3.17 (s, 1H), 2.26 (s, 6H), 1.70- 1.48 (m, 4H), 1.20 (dd, J = 20.0, 6.8 Hz, 6H), 0.95 (t, J = 7.6 Hz, 6H). |
| 32 | 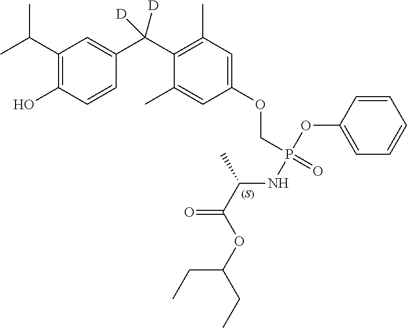 | δ 9.10 (s, 1H), 7.44-7.35 (m, 2H), 7.21-7.09 (m, 3H), 7.02-6.92 (m, 2H), 6.74 (d, J = 8.8 Hz, 1H), 5.17 (d, J = 9.4 Hz, 1H), 4.80- 4.70 (m, 1H), 4.43-4.30 (m, 2H), 3.97-3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 33 | 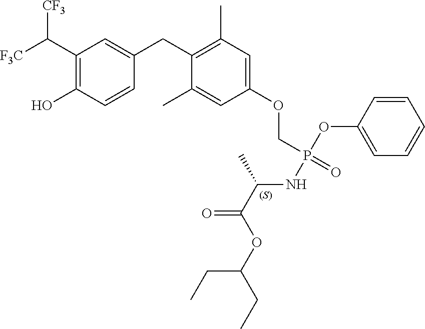 | δ 9.10 (s, 1H), 7.44-7.35 (m, 2H), 7.22-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80-4.70 (m, 1H), 4.52(s, 1H), 4.43-4.30 (m, 2H), 3.97- 3.83 (m, 3H), 2.26 (s, 6H), 1.70- 1.48 (m, 4H), 1.20 (dd, J = 20.0, 6.8 Hz, 6H), 0.95 (t, J = 7.6 Hz, 6H). |
| 34 | 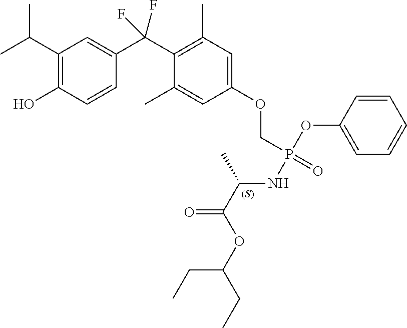 | δ 9.10 (s, 1H), 7.44-7.35 (m, 2H), 7.21-7.09 (m, 3H), 7.04-6.92 (m, 2H), 6.74 (d, J = 8.8 Hz, 1H), 5.17 (d, J = 9.4 Hz, 1H), 4.80- 4.70 (m, 1H), 4.43-4.30 (m, 2H), 3.97-3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 35 | 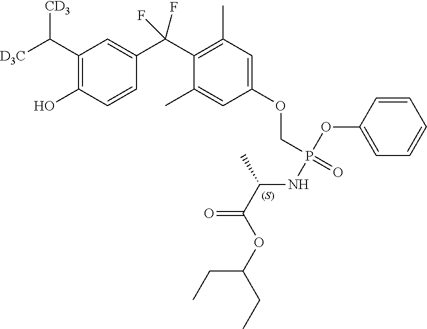 | δ 9.10 (s, 1H), 7.44-7.35 (m, 2H), 7.21-7.09 (m, 3H), 7.04-6.92 (m, 2H), 6.75 (d, J = 8.8 Hz, 1H), 5.17 (d, J = 9.4 Hz, 1H), 4.80- 4.70 (m, 1H), 4.43-4.30 (m, 2H), 3.97-3.83 (m, 3H), 3.10 (s, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 36 | 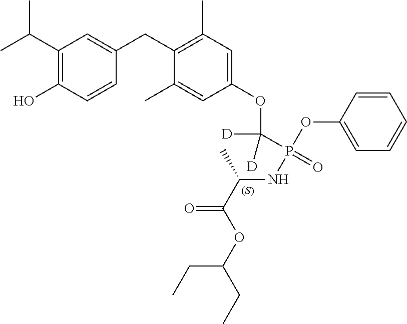 | δ 9.10 (s, 1H), 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80-4.70 (m, 1H), 3.97- 3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 37 | 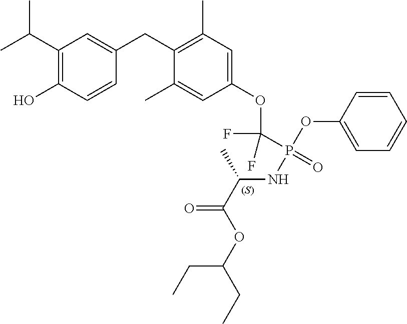 | δ 9.10 (s, 1H), 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.41-4.36 (m, 1H), 3.97- 3.83 (m, 3H), 3.17-3.07 (m, 1H), 2.26 (s, 6H), 1.70-1.48 (m, 4H), 1.35 (d, J = 7.1 Hz, 3H), 1.20 (dd, J = 20.0, 6.8 Hz, 6H), 0.95 (t, J = 7.6 Hz, 6H). |
| 38 | 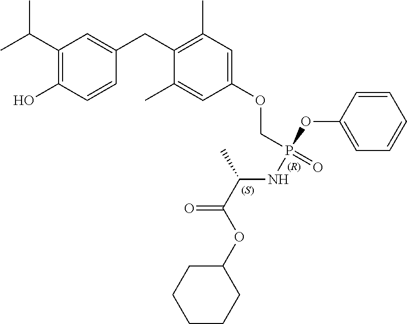 | δ 9.10 (s, 1H), 7.44-7.35 (m, 2H), 7.20-7.10 (m, 3H), 7.02-6.92 (m, 2H), 6.74 (d, J = 8.8 Hz, 1H), 6.61 (s, 2H), 5.17 (d, J = 9.4 Hz, 1H), 4.80-4.71 (m, 1H), 4.43- 4.32 (m, 2H), 3.92-3.80 (m, 3H), 3.20-3.05 (m, 1H), 2.26 (s, 6H), 1.92-1.78 (m, 2H), 1.75-1.60 (m, 4H), 1.59-1.37 (m, 4H), 1.34 (d, J = 7.2 Hz, 3H), 1.22 (d, J = 6.9 Hz, 3H), 1.17 (d, J = 6.9 Hz, 3H). |
| 39 | 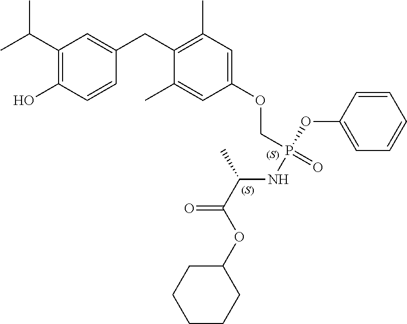 | δ 9.10 (s, 1H), 7.44-7.35 (m, 2H), 7.20-7.10 (m, 3H), 7.02-6.92 (m, 2H), 6.74 (d, J = 8.8 Hz, 1H), 6.61 (s, 2H), 5.17 (d, J = 9.4 Hz, 1H), 4.80-4.71 (m, 1H), 4.43- 4.36 (m, 2H), 3.97-3.85 (m, 3H), 3.20-3.05 (m, 1H), 2.26 (s, 6H), 1.92-1.78 (m, 2H), 1.75-1.60 (m, 4H), 1.59-1.37 (m, 4H), 1.34 (d, J = 7.2 Hz, 3H), 1.22 (d, J = 6.9 Hz, 3H), 1.17 (d, J = 6.9 Hz, 3H). |
| 40 | 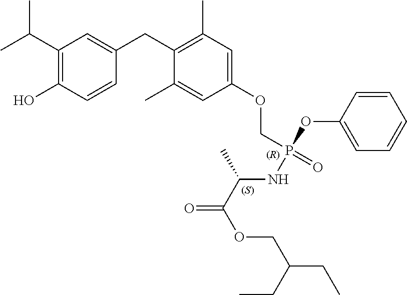 | δ 9.11 (s, 1H), 7.44-7.39 (m, 2H), 7.21-7.12 (m, 3H), 7.02-6.92 (m, 2H), 6.75 (d, J = 8.8 Hz, 1H), 6.60 (s, 2H), 5.17 (d, J = 9.4 Hz, 1H), 4.80-4.72 (m, 1H), 4.43- 4.32 (m, 2H), 3.97-3.83 (m, 3H), 3.17-3.07 (m, 1H), 2.26 (s, 6H), 1.70-1.48 (m, 5H), 1.42-1.39 (m, 4H) , 1.35 (d, J = 7.1 Hz, 3H), 1.20 (dd, J = 20.0, 6.8 Hz, 6H), 0.95 (t, J = 7.6 Hz, 6H). |
| 41 | 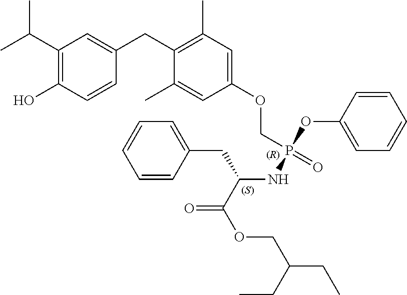 | δ 8.96 (s, 1H), 7.35-7.29 (m, 2H), 7.16-7.09 (m, 3H), 6.80-6.79 (m, 1H), 6.84 (s, 2H), 6.57-6.59 (m, 1H), 6.43-6.41 (m, 1H), 5.88- 5.72 (m, 1H), 4.62-4.58 (m, 1H), 4.33-4.30 (m, 2H), 4.02-4.01 (m, 2H), 3.77 (s, 2H), 3.11-3.07 (m, 1H), 2.13 (s, 6H), 1.55-1.53 (m, 1H), 1.52-1.36 (m, 4H), 1.20-1.09 (m, 3H), 1.05 (d,J = 6.8Hz, 6H), 0.77-0.73 (m, 6H) |
| 42 | 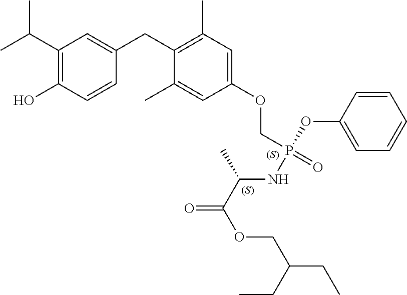 | δ 8.96 (s, 1H), 7.35-7.29 (m, 2H), 7.16-7.09 (m, 5H), 7.07-7.02 (m, 3H), 6.80-6.79 (m, 1H), 6.84 (s, 2H), 6.57-6.59 (m, 1H), 6.43-6.41 (m, 1H), 5.88-5.72 (m, 1H), 4.62- 4.58 (m, 1H), 4.33-4.30 (m, 2H), 4.02-4.01 (m, 2H), 3.77 (s, 2H), 3.11-3.07 (m, 1H), 2.13 (s, 6H), 1.55-1.53 (m, 1H), 1.52-1.36 (m, 4H), 1.20-1.09 (m, 3H), 1.05 (d, J = 6.8Hz, 6H), 0.77-0.73 (m, 6H) |
| 43 | 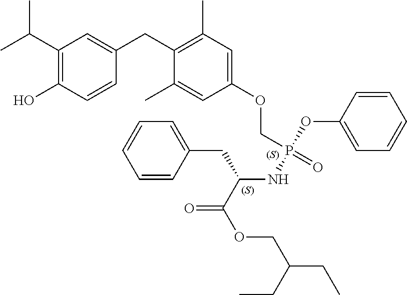 | δ 8.96 (s, 1H), 7.35-7.29 (m, 2H), 7.16-7.09 (m, 5H), 7.07-7.02 (m, 3H), 6.80-6.79 (m, 1H), 6.84 (s, 2H), 6.57-6.59 (m, 1H), 6.43-6.41 (m, 1H), 5.88-5.72 (m, 1H), 4.62- 4.58 (m, 1H), 4.33-4.30 (m, 2H), 4.02-4.01 (m, 2H), 3.77 (s, 2H), 3.11-3.07 (m, 1H), 2.13 (s, 6H), 1.55-1.53 (m, 1H), 1.52-1.36 (m, 4H), 1.20-1.09 (m, 3H), 1.05 (d, J = 6.8Hz, 6H), 0.77-0.73 (m, 6H) |
| 44 | 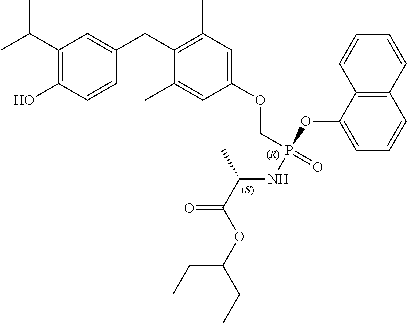 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 4.62-4.58 (m, 1H), 4.33- 4.30 (m, 2H), 4.02-4.01 (m, 1H), 3.77 (s, 2H), 3.11-3.07 (m, 1H), 2.13 (s, 6H), 1.52-1.36 (m, 4H), 1.20-1.09 (m, 3H), 1.05 (d, J = 6.8 Hz, 6H), 0.77-0.73 (m, 6H) |
| 45 | 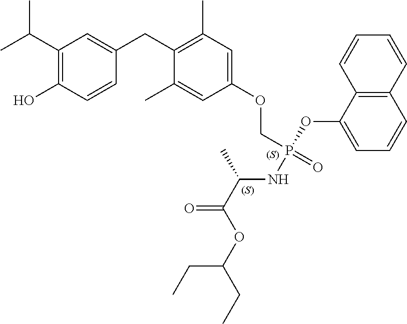 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 4.62-4.58 (m, 1H), 4.33- 4.30 (m, 2H), 4.02-4.01 (m, 1H), 3.77 (s, 2H), 3.11-3.07 (m, 1H), 2.13 (s, 6H), 1.52-1.36 (m, 4H), 1.20-1.09 (m, 3H), 1.05 (d, J = 6.8 Hz, 6H), 0.77-0.73 (m, 6H) |
| 46 | 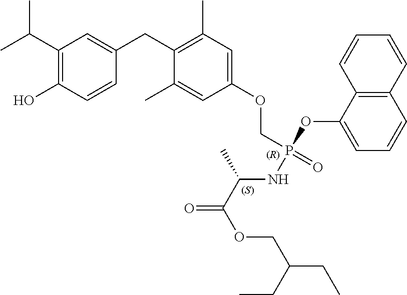 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 5.17 (d, J = 9.4 Hz, 1H), 4.80-4.72 (m, 1H), 4.43-4.32 (m, 2H), 3.97-3.83 (m, 3H), 3.17- 3.07 (m, 1H), 2.26 (s, 6H), 1.70- 1.48 (m, 5H), 1.42-1.39 (m, 4H), 1.35 (d, J = 7.1 Hz, 3H), 1.20 (dd, J = 20.0, 6.8 Hz, 6H), 0.95 (t, J = 7.6 Hz, 6H). |
| 47 | 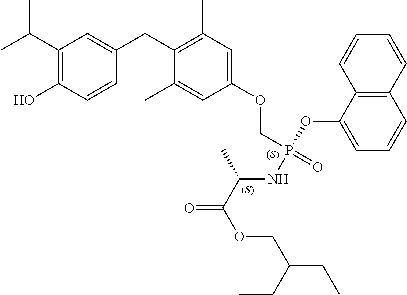 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 5.17 (d, J = 9.4 Hz, 1H), 4.80-4.72 (m, 1H), 4.43-4.32 (m, 2H), 3.97-3.83 (m, 3H), 3.17- 3.07 (m, 1H), 2.26 (s, 6H), 1.70- 1.48 (m, 5H), 1.42-1.39 (m, 4H), 1.35 (d, J = 7.1 Hz, 3H), 1.20 (dd, J = 20.0, 6.8 Hz, 6H), 0.95 (t, J = 7.6 Hz, 6H). |
| 48 | 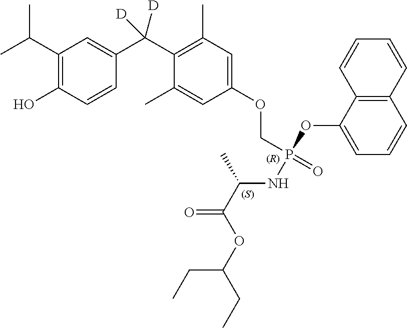 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 5.17 (d, J = 9.4 Hz, 1H), 4.80-4.72 (m, 1H), 4.43-4.32 (m, 2H), 3.90-3.83 (m, 1H), 3.17- 3.07 (m, 1H), 2.26 (s, 6H), 1.70- 1.48 (m, 5H), 1.42-1.39 (m, 4H), 1.35 (d, J = 7.1 Hz, 3H), 1.20 (dd, J = 20.0, 6.8 Hz, 6H), 0.95 (t, J = 7.6 Hz, 6H). |
| 49 | 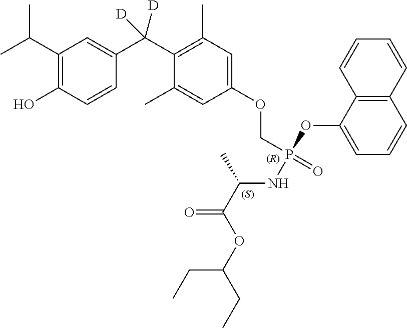 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.61 (s, 2H), 5.17 (d, J = 9.4 Hz, 1H), 4.80-4.72 (m, 1H), 4.43-4.32 (m, 2H), 3.90-3.83 (m, 1H), 3.17- 3.07 (m, 1H), 2.26 (s, 6H), 1.70- 1.48 (m, 5H), 1.42-1.39 (m, 4H), 1.35 (d, J = 7.1 Hz, 3H), 1.20 (dd, J = 20.0, 6.8 Hz, 6H), 0.95 (t, J = 7.6 Hz, 6H). |
| 50 | 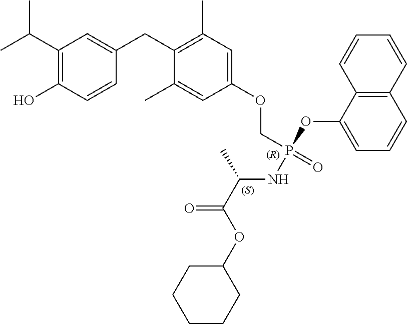 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.57- 6.59 (m, 1H), 6.43-6.41 (m, 1H), 5.88-5.72 (m, 1H), 4.62-4.58 (m, 1H), 4.33-4.30 (m, 2H), 4.02-4.01 (m, 1H), 3.77 (s, 2H), 3.11-3.07 (m, 1H), 2.13 (s, 6H), 1.72-1.55 (m, 4H), 1.25-1.11 (m, 9H), 1.05 (d, J = 6.8Hz, 6H) |
| 51 | 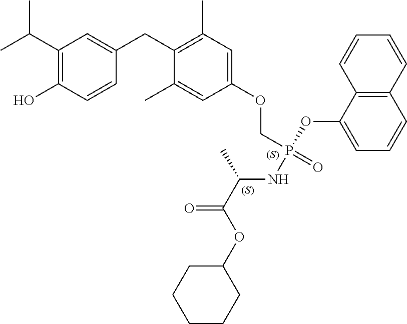 | δ 8.97 (s, 1H), 8.26-8.19 (m, 1H), 7.85-7.78 (m, 1H), 7.73-7.67 (m, 1H), 7.60-7.40 (m, 3H), 7.25- 7.19 (m, 1H), 7.00-6.90 (m, 2H), 6.74 (d, J = 8.8 Hz, 1H), 6.57- 6.59 (m, 1H), 6.43-6.41 (m, 1H), 5.88-5.72 (m, 1H), 4.62-4.58 (m, 1H), 4.33-4.30 (m, 2H), 4.02-4.01 (m, 1H), 3.77 (s, 2H), 3.11-3.07 (m, 1H), 2.13 (s, 6H), 1.72-1.55 (m, 4H), 1.25-1.11 (m, 9H), 1.05 (d, J = 6.8Hz, 6H) |
| 52 | 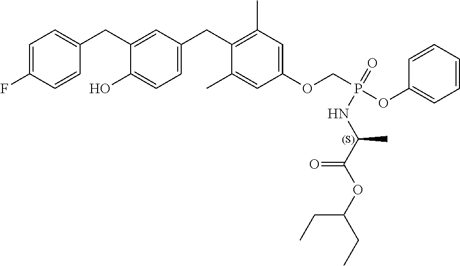 | δ 9.15 (s, 1H), 7.34-7.29 (m, 2H), 7.20-7.11 (m, 5H), 7.04-6.96 (m, 2H), 6.71-6.61 (m, 4H), 6.52- 6.49 (m, 1H), 5.90-5.81 (m, 1H), 4.61-4.53 (m, 1H), 4.33-4.25 (m, 2H), 4.03-3.96 (m, 1H), 3.73 (s, 6H), 2.11-2.02 (m,5H), 1.53- 1.33 (m, 4H), 1.20-1.18 (m, 2H), 0.83-0.69 (m, 6H) |
| 53 | 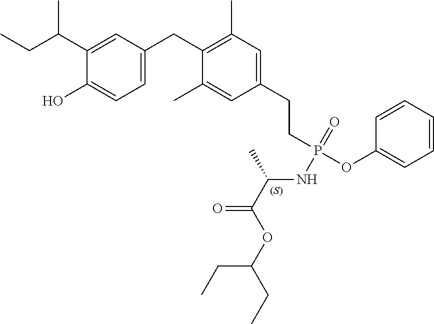 | δ 8.85 (s, 1H), 7.44-7.39 (m, 2H), 7.21-7.12 (m, 3H), 7.02-6.92 (m, 2H), 6.75 (d, J = 8.8 Hz, 1H), 6.72 (s, 2H), 5.17 (d, J = 9.4 Hz, 1H), 4.80-4.70 (m, 1H), 3.97- 3.83 (m, 3H), 3.17-2.89 (m, 3H), 2.36-2.33 (m, 2H), 2.26 (s, 6H), 1.70-1.48 (m, 4H), 1.35 (d, J = 7.1 Hz, 3H), 1.22-1.15 (m, 5H), 0.95 (t, J = 7.6 Hz, 6H), 0.83 (t, J = 4.8 Hz, 3H) |
| 72 | 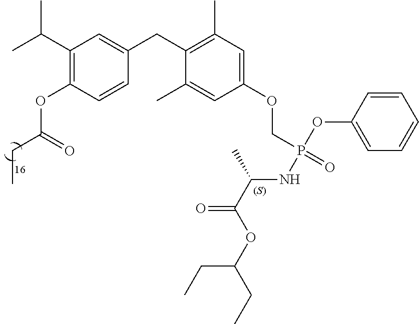 | δ 7.44-7.35 (m, 2H), 7.21-7.09 (m, 3H), 7.02-6.92 (m, 2H), 6.74 (d, J = 8.8 Hz, 1H), 6.61 (s, 2H), 5.17 (d, J = 9.4 Hz, 1H), 4.80- 4.70 (m, 1H), 4.43-4.30 (m, 2H), 3.97-3.83 (m, 3H), 3.17-3.07 (m, 1H), 2.60-2.50 (m, 2H), 2.26 (s, 6H), 1.70-1.48 (m, 6H), 1.44- 1.20 (m, 37H), 0.95-0.93 (m, 9H). |

### Example 14 Preparation of 4-(4-(((2R,4S)-4-(3-chlorophenyl)-2-oxido-1,3,2-dioxaphosphinan-2-yl)methoxy)-2,6-dimethylbenzyl)-2-isopropylphenyl stearate (Compound 54)

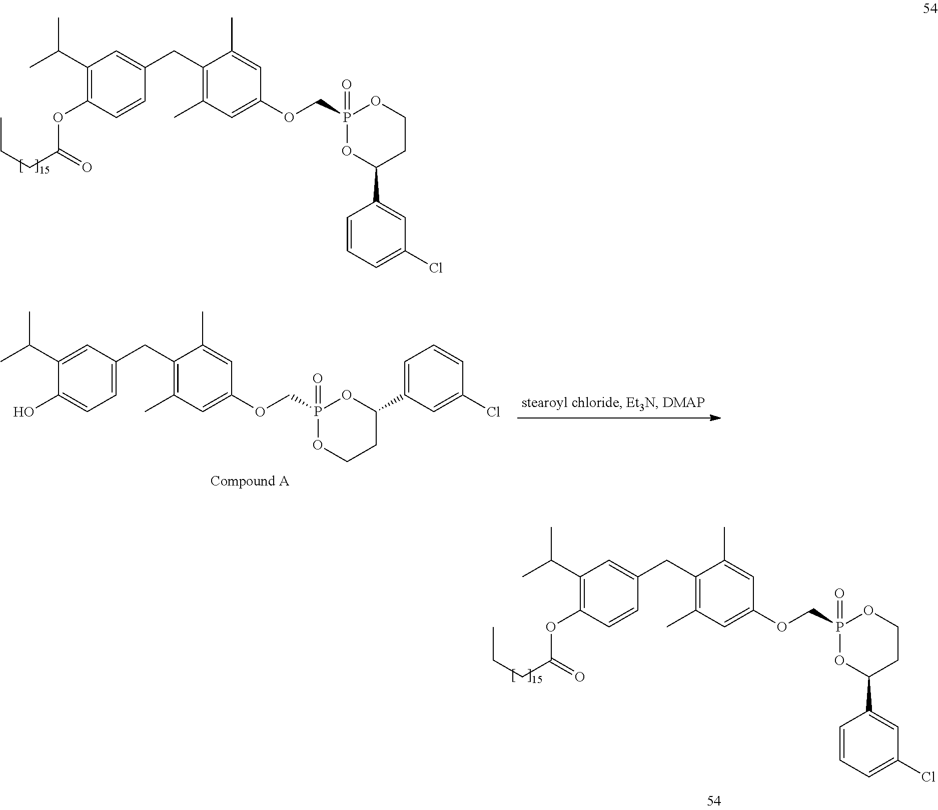

Stearoyl chloride (5.0 g, 16.5 mmol), Triethylamine (1.67 g, 16.5 mmol) and DMAP (2 mg) were added to a solution of Compound A (4.25 g, 8.25 mmol) in dichloromethane (50 ml) at 0° C. and stirred overnight at room temperature. Water (50 ml) was added into the mixture, and an organic layer was extracted. The organic layer was concentrated in vacuo to obtain a solid, which was purified by flash column chromatography (DCM/MeOH 10:1) to produce Compound 54, 4.6 g, yield:71.3%.

### Example 15. Preparation of 4-(4-(((2R,4S)-4-(3-chlorophenyl)-2-oxido-1,3,2-dioxaphosphinan-2-yl)methoxy)-2,6-dimethylbenzyl)-2-isopropylphenyl docosyl carbonate (Compound 57)

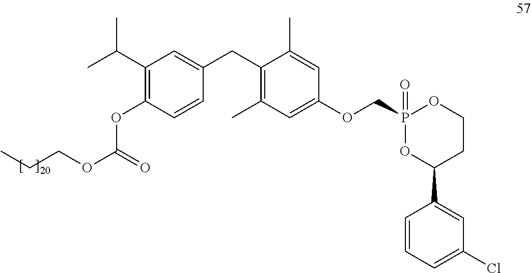

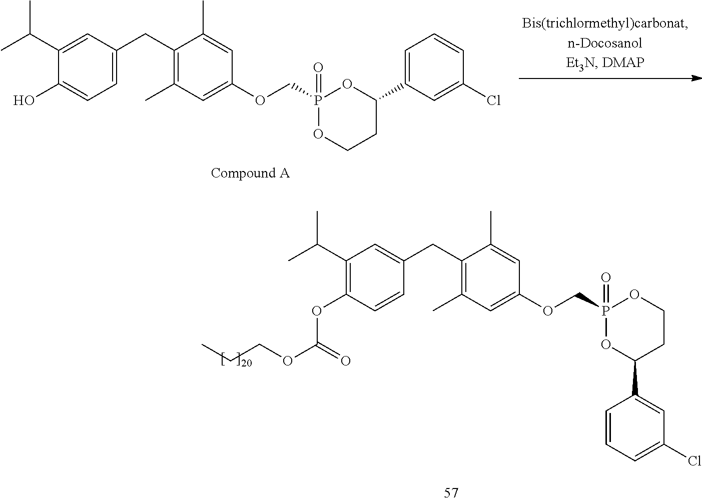

n-Docosanol (1.90 g, 5.83 mmol), Et3N (3.5 g, 35 mmol) and DMAP (2 mg) was added into dichloromethane (50 ml) at 0° C. and stirred 1 hour. Then a solution of compound A (2.0 g, 3.88 mmol, 10 ml) was added into the mixture solution, and stirred for 2 hour at 0° C. Water (50 ml) was added into the mixture, and an organic layer was extracted. The organic layer was concentrated in vacuo to obtain an oil, which was purified by flash column chromatography (DCM/MeOH 10:1) to produce Compound 57, 1.2 g, yield:35.6%.

### Example 16. Preparation of 4-(4-(((2R,4S)-4-(3-chlorophenyl)-2-oxido-1,3,2-dioxaphosphinan-2-yl)methoxy)-2,6-dimethylbenzyl)-2-isopropylphenyl L-alaninate (Compound 65)

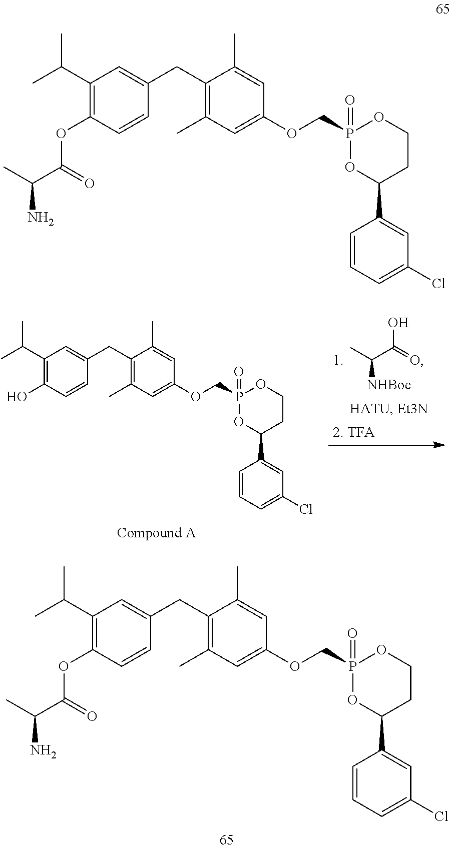

Compound A (2.5 g, 4.85 mmol), HATU (2.21 g, 5.82 mmol) and Et3N (0.74 g, 7.28 mmol) was added into dichloromethane (25 ml) at room temperature and stirred for 3 hours. Water (50 ml) was added into the mixture, and an organic layer was extracted. The organic layer was concentrated in vacuo to obtain an oil, which was added into the dichloromethane (25 ml). Then Trifluoroacetic acid (6 ml) was added into the solution and stirred for 2 hours, then a white solid was precipitated. After filtration, the compound 65 was obtained, 2.6 g, yield:91.4%.

### Example 17. Preparation of 4-(4-(((2R,4S)-4-(3-chlorophenyl)-2-oxido-1,3,2-dioxaphosphinan-2-yl)methoxy)-2,6-dimethylbenzyl)-2-isopropylphenyl 2-(((decyloxy)carbonyl)disulfaneyl)acetate (Compound 70)

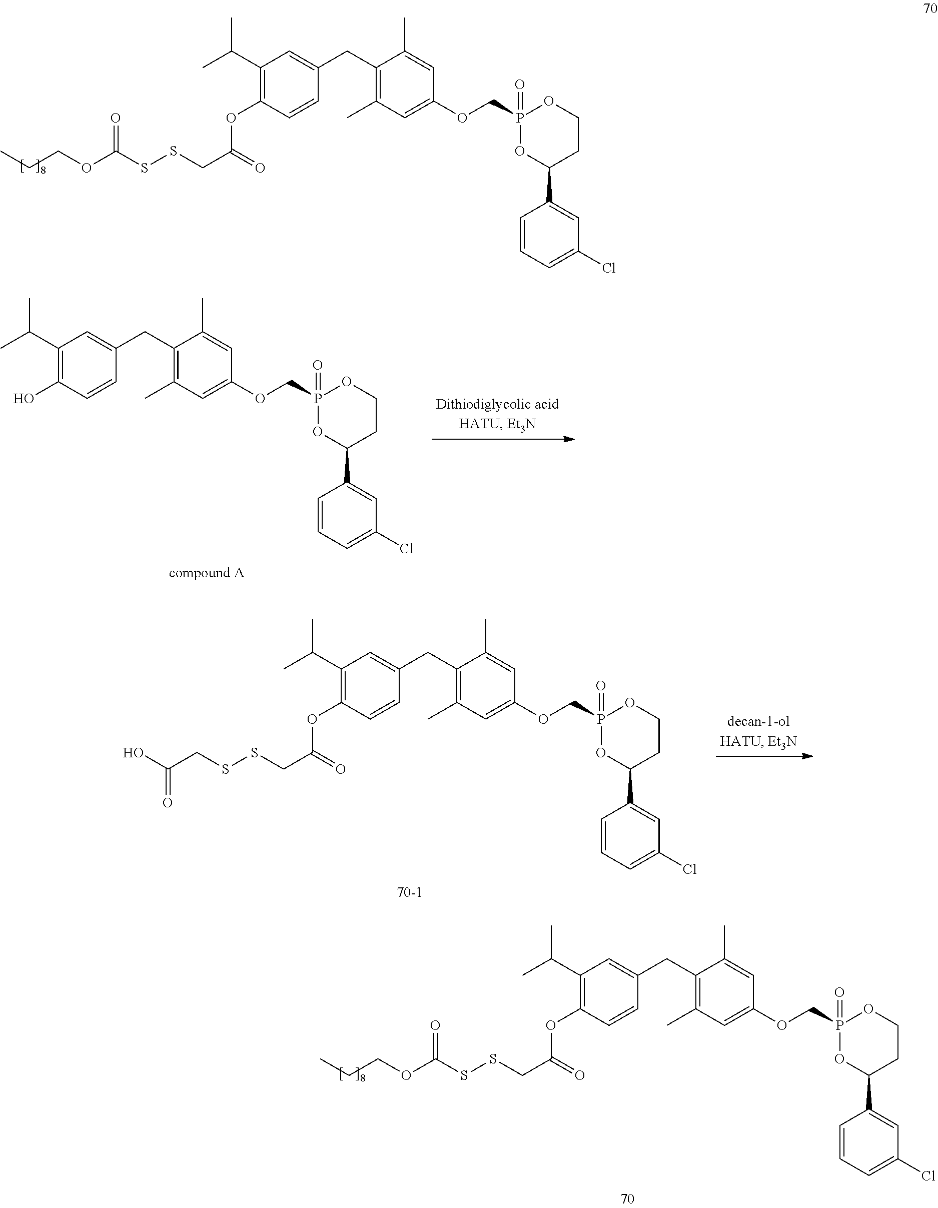

Compound A (5.14 g, 10 mmol), Dithiodiglycolic Acid (3.64 g, 20 mmol), HATU (3.8 g, 10 mmol) and Et3N (2.02 g, 20 mmol) was added into dichloromethane (50 ml) at room temperature and stirred for 3 hours. Water (50 ml) was added into the mixture, and an organic layer was extracted. The organic layer was concentrated in vacuo to obtain an oil, which was purified by flash column chromatography (DCM/MeOH 8:1) to produce Compound 70-1, 2.3 g, yield: 33.9%.

Compound 70-1 (2.0 g, 2.95 mmol), HATU (1.23 g, 3.25 mmol) and Et3N (0.60 g, 5.9 mmol) was added into dichloromethane (20 ml) at room temperature and stirred for 3 hours. Water (30 ml) was added into the mixture, and an organic layer was extracted. The organic layer was concentrated in vacuo to obtain an oil, which was purified by flash column chromatography (DCM/MeOH 15:1) to produce Compound 70, 1.5 g, yield: 63.3%.

The following compounds (in Table 2) were prepared according to the procedures described herein using the appropriate starting material(s) and appropriate protecting group chemistry as needed, and characterized by ¹HNMR as follows:

TABLE 2
The Compound list.

| | | MS | |
|---|---|---|---|
| No. | Structure | [M + H]⁺ | ¹HNMR |
| 54 | 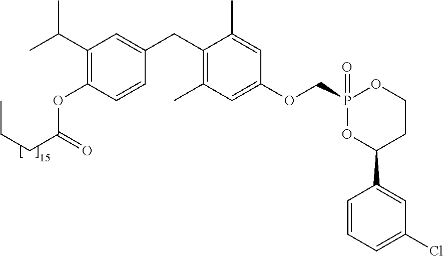 | 781.4 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.82 (s, 2H), 3.18- 3.08 (m, 1H), 2.53-2.50 (m, 2H), 2.28- 2.22 (m, 2H), 2.17 (s, 6H), 1.72- 1.69 (m, 2H), 1.15-1.0 (m, 35H), 0.91- 0.89 (m, 3H) |
| 55 | 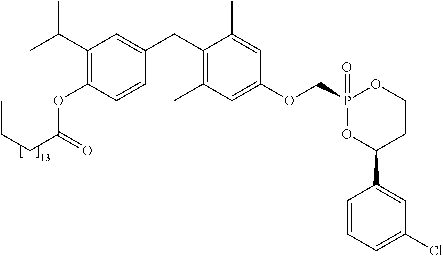 | 753.4 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.82 (s, 2H), 3.18- 3.08 (m, 1H), 2.53-2.50 (m, 2H), 2.28- 2.22 (m, 2H), 2.17 (s, 6H), 1.72- 1.69 (m, 2H), 1.15-1.0 (m, 31H), 0.91- 0.88 (m, 3H) |
| 56 | 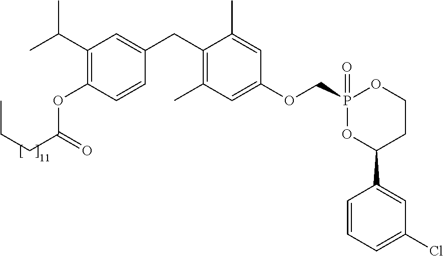 | 745.4 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.82 (s, 2H), 3.18- 3.08 (m, 1H), 2.53-2.50 (m, 2H), 2.28- 2.22 (m, 2H), 2.17 (s, 6H), 1.72- 1.68 (m, 2H), 1.15-1.0 (m, 27H), 0.91- 0.88 (m, 3H) |
| 57 | 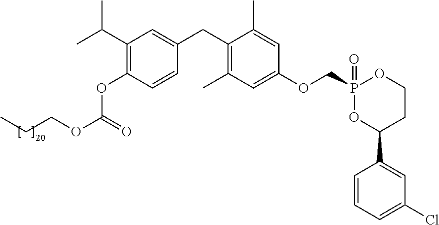 | 867.5 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 4.21-4.18 (m, 2H), 3.82 (s, 2H), 3.18-3.08 (m, 1H), 2.28- 2.22 (m, 2H), 2.17 (s, 6H), 1.73- 1.70 (m, 2H) , 1.33-1.18 (m, 45H) , 0.92-0.88 (m, 3H) |
| 58 | 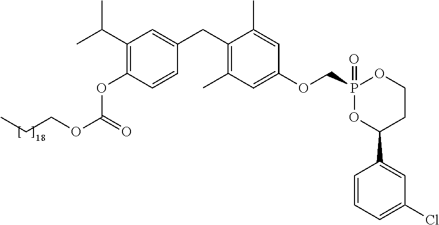 | 859.5 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 4.21-4.18 (m, 2H), 3.82 (s, 2H), 3.18-3.08 (m, 1H), 2.28- 2.22 (m, 2H), 2.17 (s, 6H), 1.73- 1.70 (m, 2H) , 1.33-1.18 (m, 41H) , 0.92-0.88 (m, 3H) |
| 59 | 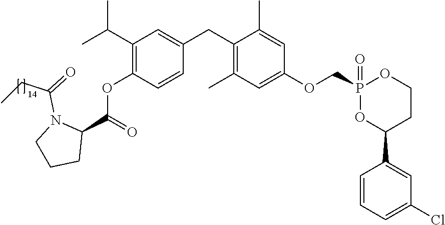 | 850.5 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 4H), 3.62-3.60 (m, 2H), 3.82 (s, 2H), 3.18-3.08 (m, 1H), 2.28- 2.20 (m, 4H), 2.17-2.15 (m, 7H), 1.92- 1.88 (m, 2H), 1.80-1.78 (m, 1H), 1.55- 1.53 (m, 2H), 1.33-1.18 (m, 31H) , 0.92-0.89 (m, 3H) |
| 60 | 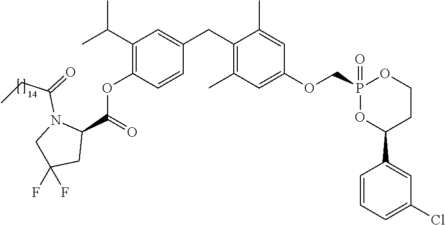 | 886.4 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 4H), 3.72-3.69 (m, 2H), 3.82 (s, 2H), 3.18-3.08 (m, 1H), 2.80- 2.78 (m, 2H), 2.28-2.20 (m, 4H), 2.17-2.15 (m, 6H), 1.55-1.53 (m, 2H), 1.33-1.18 (m, 31H) , 0.92-0.89 (m, 3H) |
| 61 | 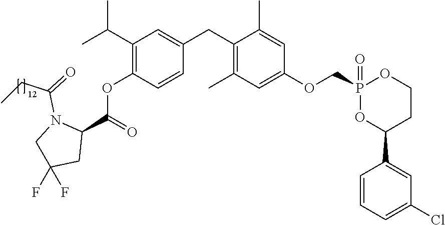 | 878.4 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 4H), 3.72-3.69 (m, 2H), 3.82 (s, 2H), 3.18-3.08 (m, 1H), 2.80- 2.78 (m, 2H), 2.28-2.20 (m, 4H), 2.17-2.15 (m, 6H), 1.55-1.53 (m, 2H), 1.33-1.18 (m, 23H) , 0.92-0.89 (m, 3H) |
| 62 | 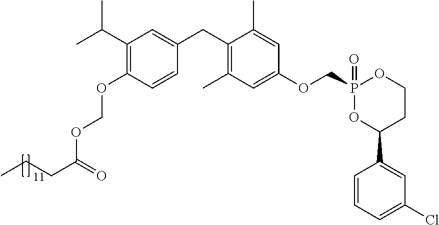 | 755.4 | 1H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 5.65-5.63 (s, 2H), 4.63- 4.56 (m, 1H), 4.50-4.43 (m, 3H), 3.82 (s, 2H), 3.18-3.08 (m, 1H), 2.53- 2.50 (m, 2H), 2.28-2.22 (m, 2H), 2.17 (s, 6H), 1.72-1.69 (m, 2H), 1.15- 1.0 (m, 27H), 0.91-0.89 (m, 3H) |
| 63 | 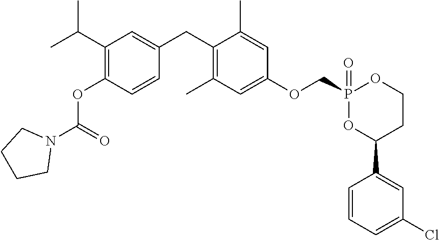 [SMILES: Cc1cc(OC[P@@]2(=O)OCC[C@@H](c3cccc(Cl)c3)O2)cc(C)c1Cc1ccc(OC(=O)N2CCCC2)c(C(C)C)c1] | 612.2 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.82 (s, 2H), 3.57- 3.50 (m, 4H), 3.18-3.08 (m, 1H), , 2.28-2.22 (m, 2H), 2.17 (s, 6H), 1.87- 1.84 (m, 4H), 1.12-1.0 (d, J = 2.4Hz, 6H) |
| 64 | 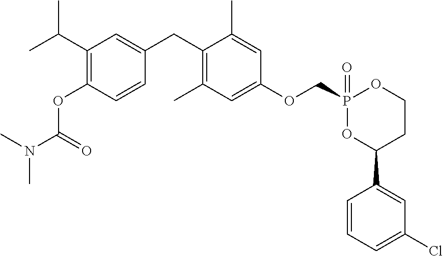 [SMILES: Cc1cc(OC[P@@]2(=O)OCC[C@@H](c3cccc(Cl)c3)O2)cc(C)c1Cc1ccc(OC(=O)N(C)C)c(C(C)C)c1] | 586.2 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.82 (s, 2H), 3.18- 3.08 (m, 1H), 2.94 (s, 6H), 2.28- 2.22 (m, 2H), 2.17 (s, 6H), 1.12-1.0 (d, J = 2.4Hz, 6H) |
| 65 | 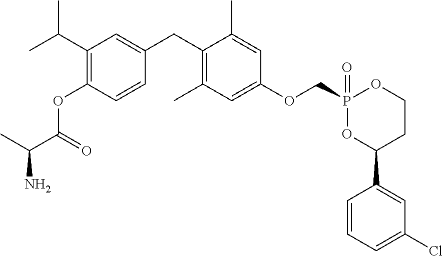 [SMILES: Cc1cc(OC[P@@]2(=O)OCC[C@@H](c3cccc(Cl)c3)O2)cc(C)c1Cc1ccc(OC(=O)[C@H](C)N)c(C(C)C)c1] | 586.2 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.85-3.82 (m, 3H), 3.18- 3.08 (m, 1H), 2.28-2.22 (m, 2H), 2.17 (s, 6H), 1.54 (d, J = 4.0Hz, 3H), 1.12-1.0 (d, J = 2.4Hz, 6H) |
| 66 | 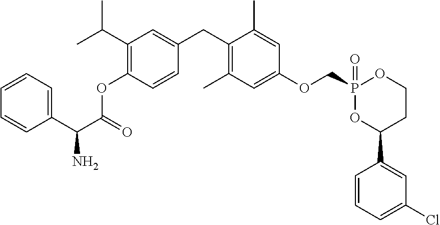 [SMILES: Cc1cc(OC[P@@]2(=O)OCC[C@@H](c3cccc(Cl)c3)O2)cc(C)c1Cc1ccc(OC(=O)[C@@H](N)c2ccccc2)c(C(C)C)c1] | 648.2 | ¹H NMR (400M, d-DMSO) δ: 7.50 (s, 1H), 7.45-7.43 (m, 2H), 7.39-7.34 (m, 5H), 7.31-7.29 (m, 1H), 6.85 (d, J = 6.4Hz, 1H), 6.76 (s, 1H), 6.63- 6.61 (m, 1H), 6.48-6.45 (m, 1H), 5.76- 5.74 (m, 1H), 5.12-5.09 (m, 1H), 4.63- 4.56 (m, 1H), 4.50-4.43 (m, 3H), 3.82 (s, 2H), 3.18-3.08 (m, 1H), 2.28- 2.22 (m, 2H), 2.17 (s, 6H), 1.12-1.0 (d, J = 2.4Hz, 6H) |
| 67 | 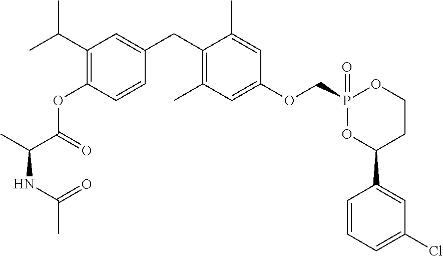 [SMILES: CC(=O)N[C@@H](C)C(=O)Oc1ccc(Cc2c(C)cc(OC[P@@]3(=O)OCC[C@@H](c4cccc(Cl)c4)O3)cc2C)cc1C(C)C] | 628.2 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.85-3.82 (m, 3H), 3.18- 3.08 (m, 1H), 2.28-2.22 (m, 2H), 2.17 (s, 6H), 1.95 (s, 3H), 1.54 (d, J = 4.0Hz, 3H), 1.12-1.0 (d, J-2.4Hz, 6H) |
| 68 | 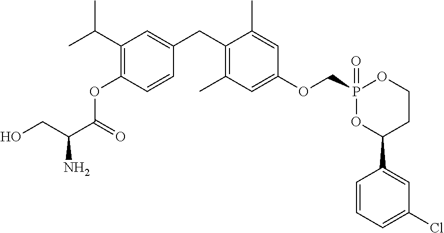 [SMILES: Cc1cc(OC[P@@]2(=O)OCC[C@@H](c3cccc(Cl)c3)O2)cc(C)c1Cc1ccc(OC(=O)[C@@H](N)CO)c(C(C)C)c1] | 602.2 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.85-3.82 (m, 3H), 3.18- 3.08 (m, 1H), 2.28-2.22 (m, 2H), 2.17 (s, 6H), 1.86-1.82 (m, 2H), 1.12- 1.0 (d, J = 2.4Hz, 6H) |
| 69 | 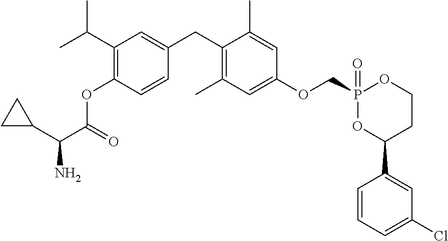 [SMILES: Cc1cc(OC[P@@]2(=O)OCC[C@@H](c3cccc(Cl)c3)O2)cc(C)c1Cc1ccc(OC(=O)[C@@H](N)C2CC2)c(C(C)C)c1] | 612.2 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 3.85-3.82 (m, 3H), 3.18- 3.08 (m, 1H), 2.28-2.22 (m, 2H), 2.17 (s, 6H), 1.43 (m, 1H), 1.12-1.0 (d, J = 2.4Hz, 6H), 0.98-0.91 (m, 4H) |
| 70 | 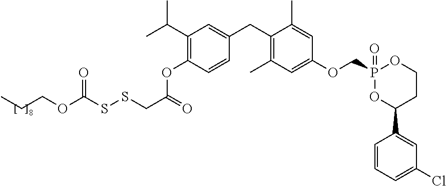 | 804.3 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 4.63-4.56 (m, 1H), 4.50- 4.43 (m, 3H), 4.19-4.17 (m, 2H), 3.97- 3.95 (m, 2H), 3.82 (s, 2H), 3.18- 3.08 (m, 1H), 2.28-2.22 (m, 2H), 2.17 (s, 6H), 1.68-1.66 (m, 2H), 1.32- 1.18 (m, 21H), 0.92-0.89 (m, 3H) |
| 71 | 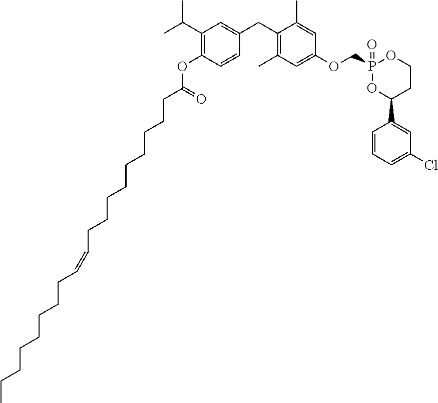 [SMILES: CCCCCCCC/C=C\CCCCCCCCCC(=O)Oc1ccc(Cc2c(C)cc(OC[P@@]3(=O)OCC[C@@H](c4cccc(Cl)c4)O3)cc2C)cc1C(C)C] | 779.4 | ¹H NMR (400M, d-DMSO) δ: 7.50 (s, 1H), 7.39-7.34 (m, 3H), 6.85 (d, J = 6.4Hz, 1H), 6.76 (s, 1H), 6.63- 6.61 (m, 1H), 6.48-6.45 (m, 1H), 5.76- 5.74 (m, 1H), 5.33-5.31 (m, 2H), 4.63- 4.56 (m, 1H), 4.50-4.43 (m, 3H), 3.82 (s, 2H), 3.18-3.08 (m, 1H), 2.53- 2.50 (m, 2H), 2.28-2.22 (m, 2H), 2.03- 2.00 (m, 4H), 2.17 (s, 6H), 1.72- 1.69 (m, 2H), 1.20-1.0 (m, 29H), 0.91- 0.89 (m, 3H) |

### Pharmacokinetic Study

### 1. Absorption Pharmacokinetics in Rats

The compound of formula I is a prodrug of protide, which would be metabolized into an active moiety compound A or analogues.

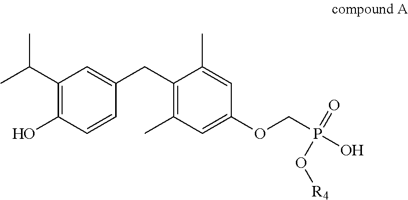

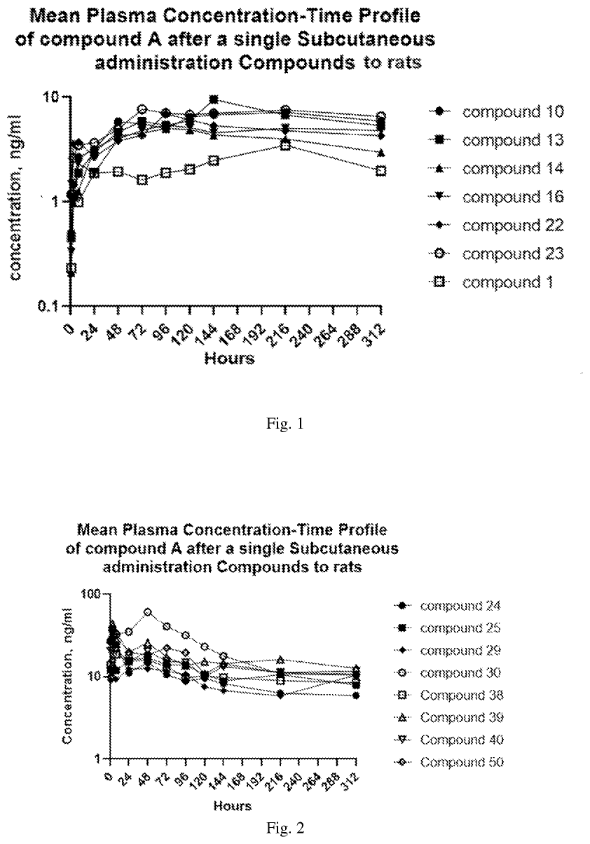

a. Individual plasma concentrations of compound A after a single subcutaneous administration of Compound 1, 10, 13, 14, 16, 22, 23, 24, 25, 26, 28, 29, 30, 38, 39, 40 and 50 to SD rat (14 mg/kg equivalent to active moiety) were used to calculate the mean pharmacokinetic parameters summarized in FIG. 1 and FIG. 2.

b. In vivo, Compound 31 would be metabolize into compound C; Compound 32 would be metabolize into compound D; Compound 34 would be metabolize into compound E; Compound 36 would be metabolize into compound F;

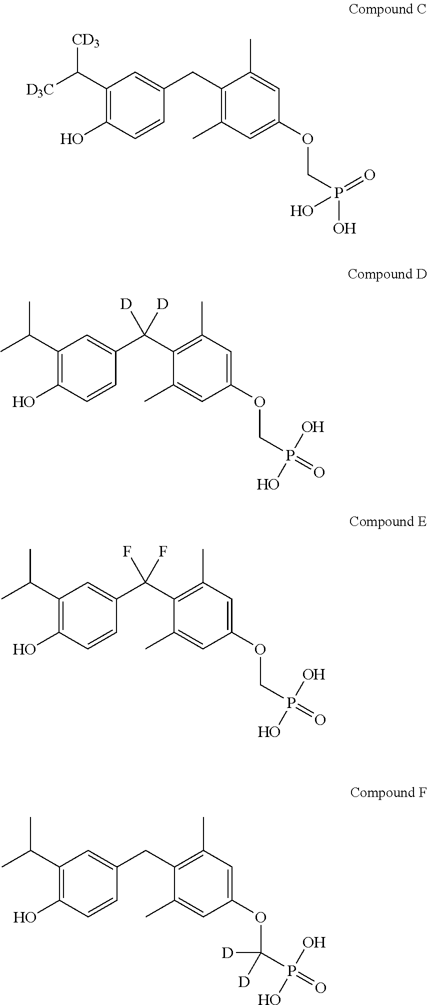 [SMILES: [2H]C([2H])([2H])C(c1cc(Cc2c(C)cc(OCP(=O)(O)O)cc2C)ccc1O)C([2H])([2H])[2H] ; [2H]C([2H])(c1ccc(O)c(C(C)C)c1)c1c(C)cc(OCP(=O)(O)O)cc1C ; Cc1cc(OCP(=O)(O)O)cc(C)c1C(F)(F)c1ccc(O)c(C(C)C)c1 ; [2H]C([2H])(Oc1cc(C)c(Cc2ccc(O)c(C(C)C)c2)c(C)c1)P(=O)(O)O]

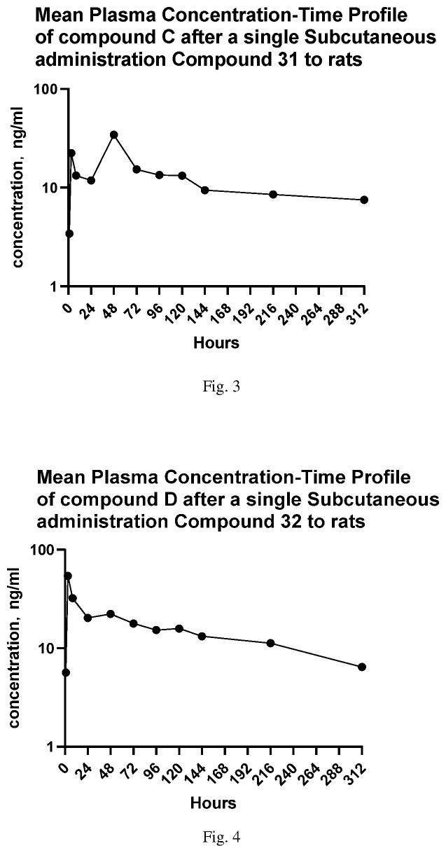

Individual plasma concentrations of compound C after a single subcutaneous administration of Compound 31 to SD rat (14 mg/kg equivalent to compound C) were used to calculate the mean pharmacokinetic parameters summarized in FIG. 3.

Individual plasma concentrations of compound D after a single subcutaneous administration of Compound 32 to SD rat (14 mg/kg equivalent to compound D) were used to calculate the mean pharmacokinetic parameters summarized in FIG. 4.

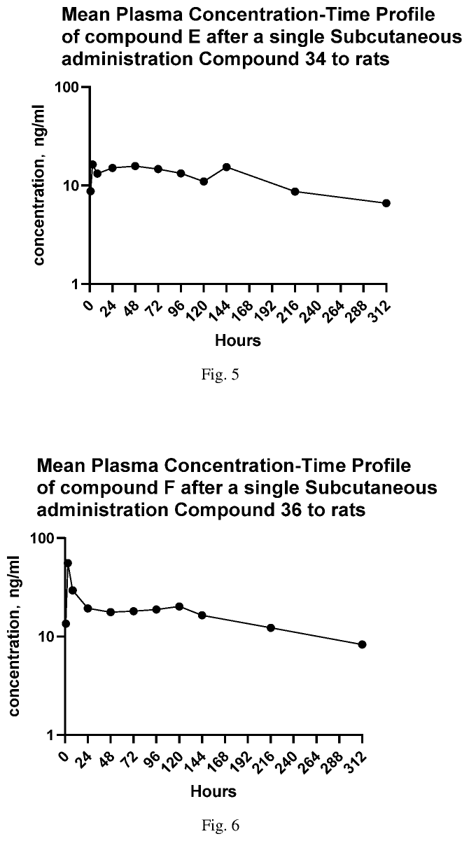

Individual plasma concentrations of compound E after a single subcutaneous administration of Compound 34 to SD rat (14 mg/kg equivalent to compound E) were used to calculate the mean pharmacokinetic parameters summarized in FIG. 5.

Individual plasma concentrations of compound F after a single subcutaneous administration of Compound 36 to SD rat (14 mg/kg equivalent to compound F) were used to calculate the mean pharmacokinetic parameters summarized in FIG. 6.

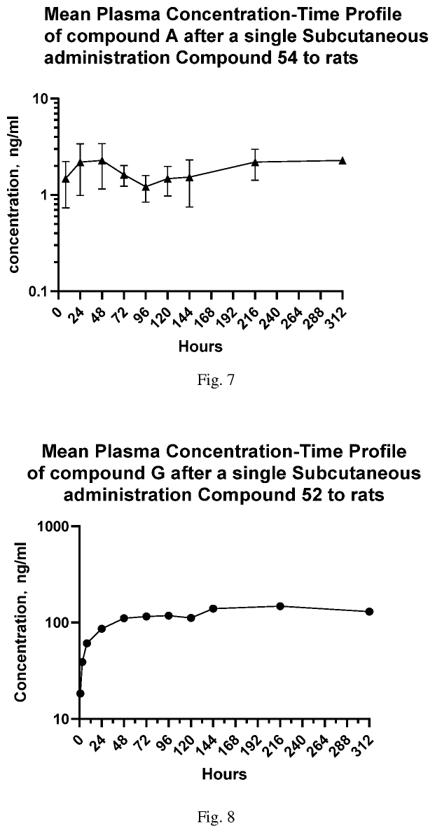

c. Compounds of formula II were prodrugs, which would be metabolized in vivo to compound A. Therefore, individual plasma concentrations of compound A after a single subcutaneous administration of compound 54 to rat (20 mg/kg) were monitored and used to calculate the mean pharmacokinetic parameters. The results are shown, in FIG. 7.

d. In vivo, Compound 52 would be metabolize into compound G; Compound 53 would be metabolize into compound H;

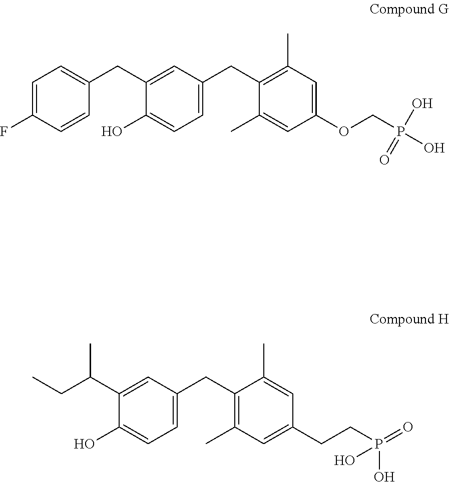 [SMILES: Cc1cc(OCP(=O)(O)O)cc(C)c1Cc1ccc(O)c(Cc2ccc(F)cc2)c1 ; CCC(C)c1cc(Cc2c(C)cc(CCP(=O)(O)O)cc2C)ccc1O]

Individual plasma concentrations of compound G after a single subcutaneous administration of Compound 52 to SD rat (14 mg/kg equivalent to compound G) were used to calculate the mean pharmacokinetic parameters summarized in FIG. 8.

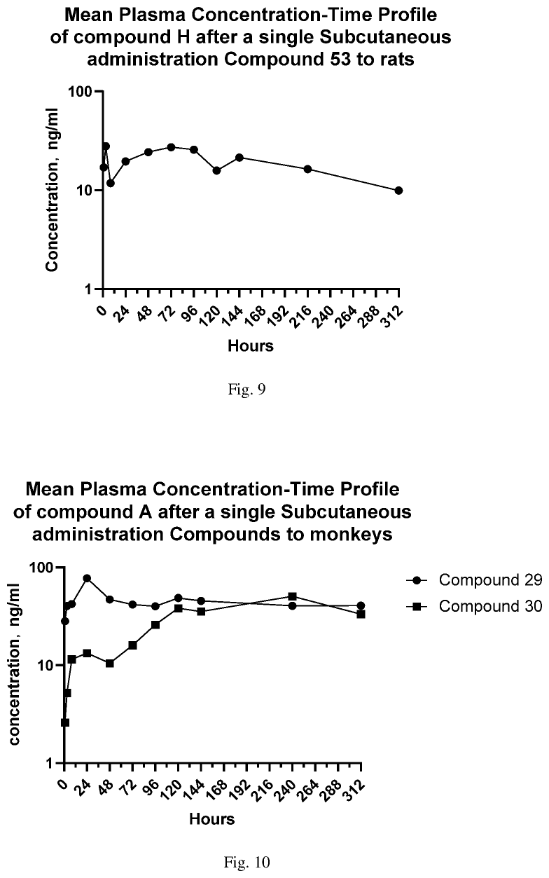

Individual plasma concentrations of compound H after a single subcutaneous administration of Compound 53 to SD rat (14 mg/kg equivalent to compound H) were used to calculate the mean pharmacokinetic parameters summarized in FIG. 9.

### 2. Absorption Pharmacokinetics in Male Cynomolgus Monkeys

a. Individual plasma concentrations of compound A after a single subcutaneous administration of Compound 29 and 30 to Male Cynomolgus Monkeys were used to calculate the mean pharmacokinetic parameters summarized in FIG. 10.

Dosing Information: Formulation concentration: 10 mg/kg; Formulation concentration: 20 mg/ml.

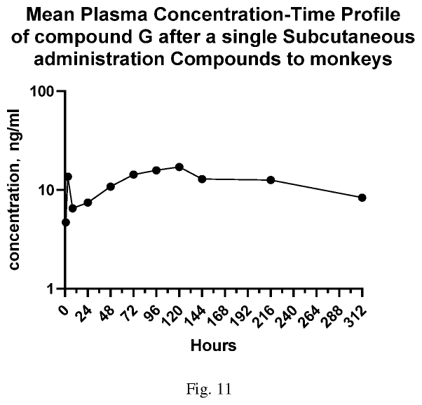

b. Individual plasma concentrations of compound G after a single subcutaneous administration of Compound 52 to Male Cynomolgus Monkeys were used to calculate the mean pharmacokinetic parameters summarized in FIG. 11.

Dosing Information: Formulation concentration: 10 mg/kg; Formulation concentration: 20 mg/ml.

### 3. Distribution Pharmacokinetics in Rats

The distribution of compound A in plasma and liver was detected after single subcutaneous administration of compounds, summarized as follows:

| | Time of | Concentration in | Concentration in | |
|---|---|---|---|---|
| compound | necropsy | plasma (ng/ml) | liver (ng/g) | L/P ration |
| 54 | D14 | 2.93 | 36.6 | 12.49 |
| 29 | D68 | 1.45 | 16.9 | 11.66 |

What is claimed is:

1. A compound of Formula I: 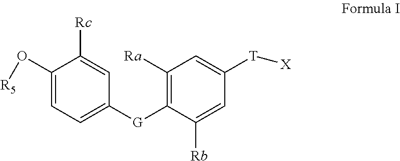
stereoisomers thereof, pharmaceutically acceptable salts thereof, deuterium substitutes thereof, isomers thereof, prodrugs thereof, and metabolites thereof,
wherein:
G is selected from the group consisting of —O—, and —C(X₉X₈)—;
T is selected from the group consisting of —(CR_{d})ₘ—, and —O—(CR_{d})ₘ—;
m is an integer from 0-3;
X is selected from the group consisting of: 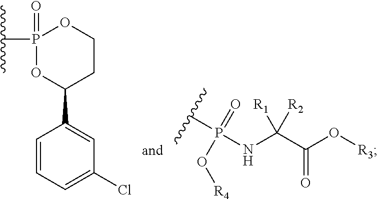
each of Ra and Rb is independently selected from the group consisting of CH₃, CD₃, Cl, Br, I and CF₃;
wherein R_{c} is selected from the group consisting of hydrogen, halogen, —CF₃, —OCF₃, cyano, optionally substituted —C₁-C₁₂ alkyl, optionally substituted —C₂-C₁₂ alkenyl, optionally substituted-C₂-C₁₂ alkynyl, optionally substituted —C₀₋₆alkyl-aryl, optionally substituted —C₀₋₆ alkyl-cycloalkyl, optionally substituted —C₀₋₆ alkyl-heterocycloalkyl, and optionally substituted —C₃₋₈ cycloalkyl;
wherein R_{c} is optionally substituted with one to ten halogen, H or D;
each of R_{d} is independently selected from the group consisting of hydrogen, halogen, and C₁₋₆ alkyl;
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —COOR₆, CH₂OC(O)OR₇, —CONHR₆, —CONR₇R₁₀, —CONR₆R₇, —CH₂OCOR₆, —CH₂OCONHR₆, and 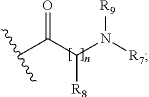
wherein each of R₆ is independently C₁₋₃₀ alkyl, C₁₋₃₀ alkenyl, and C₁₋₃₀ alkynyl, wherein C₁₋₃₀ alkyl, C₁₋₃₀ alkenyl, and C₁₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₇ is selected from the group consisting of H, —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl, wherein each of the —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein each of R₈ and R₉ is independently selected from the group consisting of H, OH, C₁₋₆ alkyl, halo, —O—C₁₋₃ alkyl, —S—C₁₋₃ alkyl, —C₃₋₁₀ cycloalkyl, 3-10 membered heterocyclyl, aryl, and 5-10 membered heteroaryl, wherein each of the C₁₋₆ alkyl, —O—C₁₋₃ alkyl, —S—C₁₋₃ alkyl, —C₃₋₁₀ cycloalkyl, 3-10 membered heterocyclyl, aryl, and 5-10 membered heteroaryl can be optionally substituted with one or more substituents each independently selected from the group consisting of OH, halo, —O—C₁₋₃ alkyl, —S—C₁₋₃ alkyl, —C₃₋₁₀ cycloalkyl, 3-10 membered heterocyclyl, aryl, and 5-10 membered heteroaryl;
or wherein R₈ and R₉ are combined with the atoms to which they are attached to form a 3-10 membered heterocyclyl, which is optionally substituted with one to four R₆, wherein each R₆ is independently H, halo, C₁₋₆ alkyl, C₁₋₆ haloalkyl, or alkoxy;
wherein R₁₀ is selected from the group consisting of H, C₁₋₆ alkyl, C₂₋₆ alkenyl, C₂₋₆ alkynyl, —(CH₂), C₃₋₁₀cycloalkyl, —(CH₂), aryl, and —(CH₂), heteroaryl, wherein each of the C₁₋₆ alkyl, C₂₋₆ alkenyl, C₂₋₆ alkynyl, —(CH₂), C₃₋₁₀cycloalkyl, —(CH₂)ₙ aryl, and —(CH₂)ₙ heteroaryl is optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein X₈ and X₉ is independently H, D or halo;
wherein n=0, 1, 2, or 3;
wherein each 5-10 membered heteroaryl mentioned above has one to four heteroatoms, and each is independently N, O, or S; and
wherein each 3-10 membered heterocyclyl mentioned above has one to four heteroatoms, and each is independently N, O, or S,
provided that the compound of formula I is not isopropyl (((4-(4-hydroxy-3-isopropylbenzyl)-3,5-dimethylphenoxy)methyl)(phenoxy)phosphoryl)-L-alaninate.

2. The compound of claim 1, stereoisomers thereof, pharmaceutically acceptable salts thereof, deuterium substitutes thereof, isomers thereof, prodrugs thereof, and metabolites thereof, wherein
G is —O—;
T is —O—(CR_{d})ₘ—;
m is an integer from 0-3;
X is 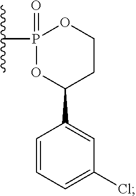 [SMILES: *P1(=O)OCC[C@@H](c2cccc(Cl)c2)O1]
R_{c} is selected from the group consisting of hydrogen, halogen, —CF₃, —OCF₃, cyano, and optionally substituted —C₁-C₁₂ alkyl; and
each of Ra is independently selected from the group consisting of hydrogen, halogen, and C₁₋₆ alkyl.

3. The compound of claim 1, stereoisomers thereof, pharmaceutically acceptable salts thereof, deuterium substitutes thereof, isomers thereof, prodrugs thereof, and metabolites thereof, wherein each of Rd is hydrogen.

4. The compound of claim 1, stereoisomers thereof, pharmaceutically acceptable salts thereof, deuterium substitutes thereof, isomers thereof, prodrugs thereof, and metabolites thereof, wherein m is 1.

5. The compound of claim 1, stereoisomers thereof, pharmaceutically acceptable salts thereof, deuterium substitutes thereof, isomers thereof, prodrugs thereof, and metabolites thereof, wherein the formula I is formula II, 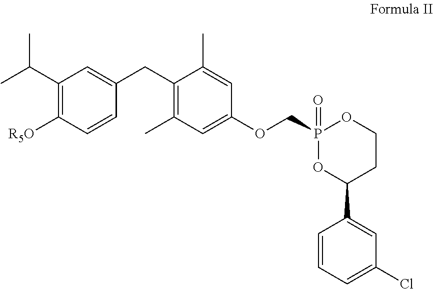
stereoisomers thereof, pharmaceutically acceptable salts thereof, and deuterium substitutes thereof,
wherein R₅ is selected from the group consisting of —COR₆, —COOR₆, CONR₇R₁₀, —CH₂OCOR₆, CH₂OC(O)OR₇, 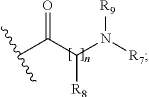
wherein each of R₆ is independently C₁₃₋₃₀ alkyl, C₁₃₋₃₀ alkenyl, and C₁₃₋₃₀ alkynyl, wherein each of the C₁₃₋₃₀ alkyl, C₁₃₋₃₀ alkenyl, and C₁₃₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein R₇ is selected from the group consisting of H, —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRCC₂₋₃₀ alkenyl, and —C(O) NRCC₂₋₃₀ alkynyl, wherein each of the —C₁₋₃₀ alkyl, —C₂₋₃₀ alkenyl, —C₂₋₃₀ alkynyl, —C(O)C₁₋₃₀ alkyl, —C(O)C₂₋₃₀ alkenyl, —C(O)C₂₋₃₀ alkynyl, —C(O)OC₁₋₃₀ alkyl, —C(O)OC₂₋₃₀ alkenyl, —C(O)OC₂₋₃₀ alkynyl, —C(O)NRCC₁₋₃₀ alkyl, —C(O) NRcC₂₋₃₀ alkenyl, and —C(O) NRCC₂₋₃₀ alkynyl is optionally substituted with one or more substituents each independently selected from the group consisting of halo, —O—C₁₋₃₀ alkyl, —S—C₁₋₃₀ alkyl, cycloalkyl, heterocyclyl, aryl, and 5-10 membered heteroaryl, wherein each of the —O—C₁₋₃₀ alkyl, —S—C₁₋₃₀ alkyl, cycloalkyl, heterocyclyl, aryl, and 5-10 membered heteroaryl is optionally substituted with one or more substituents each independently selected from the group consisting of halo and C₁₋₃ alkyl;
wherein each of R₈ and R₉ is independently selected from the group consisting of H, OH, C₁₋₆ alkyl, halo, —O—C₁₋₃ alkyl, —S—C₁₋₃ alkyl, —C₃₋₁₀ cycloalkyl, 3-10 membered heterocyclyl, aryl, and 5-10 membered heteroaryl, wherein each of the C₁₋₆ alkyl, —O—C₁₋₃ alkyl, —S—C₁₋₃ alkyl, —S—S—C(O)OC₂₋₃₀ alkyl, —C₃₋₁₀ cycloalkyl, 3-10 membered heterocyclyl, aryl, and 5-10 membered heteroaryl can be optionally substituted with one or more substituents each independently selected from the group consisting of OH, halo, —O—C₁₋₃ alkyl, —S—C₁₋₃ alkyl, —C₃₋₁₀ cycloalkyl, 3-10 membered heterocyclyl, aryl, and 5-10 membered heteroaryl;
or wherein R₈ and R₉ are combined with the atoms to which they are attached to form a 3-10 membered heterocyclyl, which is optionally substituted with one to four R₆, wherein each R₆ is independently H, halo, C₁₋₆ alkyl, C₁₋₆ haloalkyl, or alkoxy;
wherein R₁₀ is selected from the group consisting of H, C₁₋₆ alkyl, C₂₋₆ alkenyl, C₂₋₆ alkynyl, —(CH₂)ₙ C₃₋₁₀cycloalkyl, —(CH₂)ₙ aryl, and —(CH₂)ₙ heteroaryl, wherein each of the C₁₋₆ alkyl, C₂₋₆ alkenyl, C₂₋₆ alkynyl, —(CH₂)ₙ C₃₋₁₀cycloalkyl, —(CH₂), aryl, and —(CH₂)ₙ heteroaryl is optionally substituted with one or more substituents each independently selected from the group consisting of halo, —O—C₁₋₃ alkyl, —S—C₁₋₃ alkyl, —C₃₋₁₀ cycloalkyl, 3-10 membered heterocyclyl, aryl, and 5-10 membered heteroaryl;
wherein n=0, 1, 2, or 3;
wherein each 5-10 membered heteroaryl mentioned above has one to four heteroatoms, each is independently N, O, or S; and
wherein each 3-10 membered heterocyclyl mentioned above has one to four heteroatoms, each is independently N, O, or S.

6. The compound of claim 5, stereoisomers thereof, pharmaceutically acceptable salts thereof, deuterium substitutes thereof, isomers thereof, prodrugs thereof, and metabolites thereof, wherein
wherein R₅ is selected from the group consisting of —COR₆, —COOR₆, CONR₇R₁₀, —CH₂OCOR₆, and

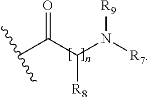

7. The compound of claim 5, stereoisomers thereof, pharmaceutically acceptable salts thereof, deuterium substitutes thereof, isomers thereof, prodrugs thereof, and metabolites thereof, wherein each of R₆ is independently C₁₃₋₃₀ alkyl and C₁₃₋₃₀ alkenyl.

8. The compound of claim 1, stereoisomers thereof, pharmaceutically acceptable salts thereof, deuterium substitutes thereof, isomers thereof, prodrugs thereof, and metabolites thereof, wherein the compound is selected from the group consisting of:

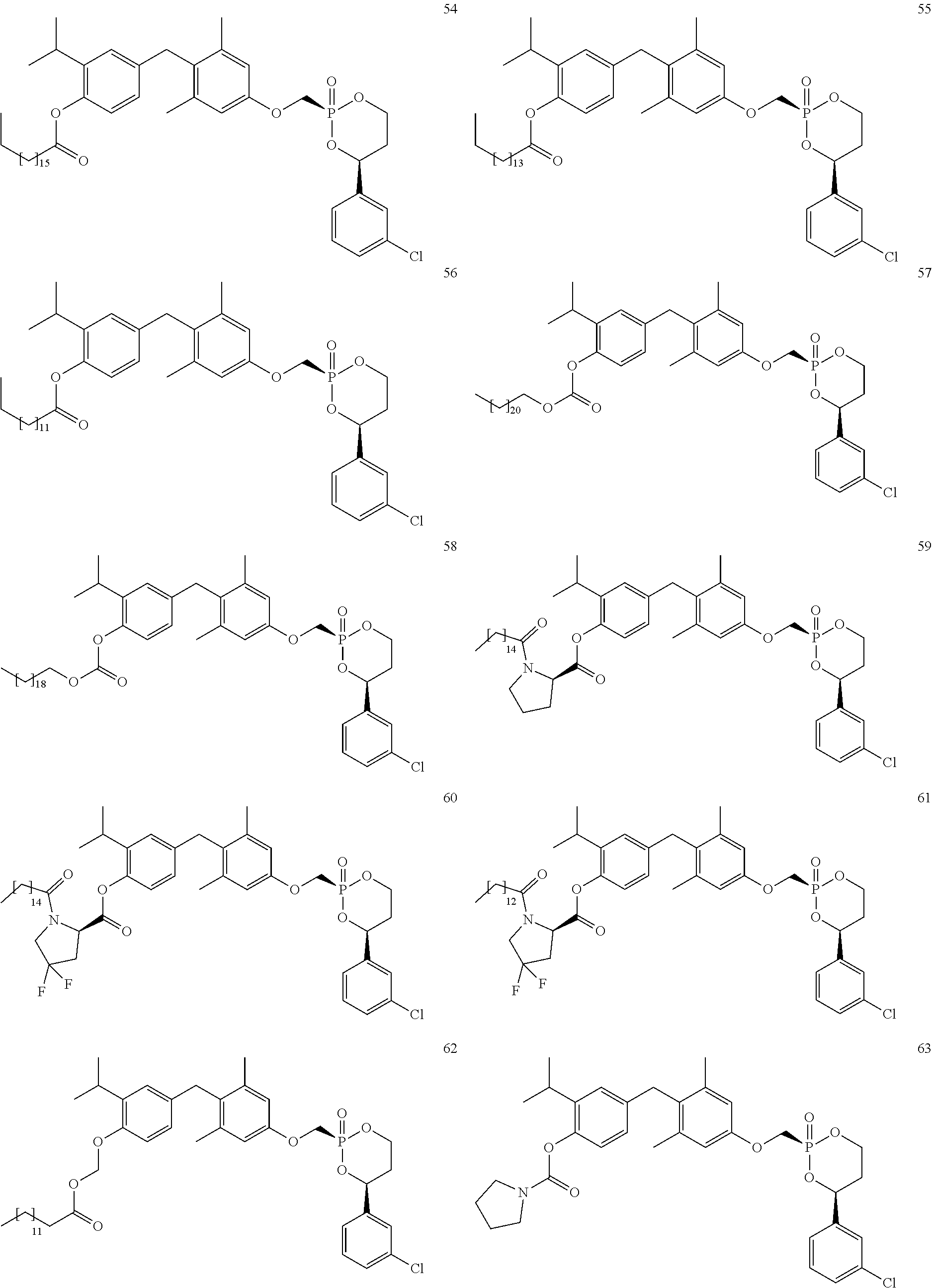

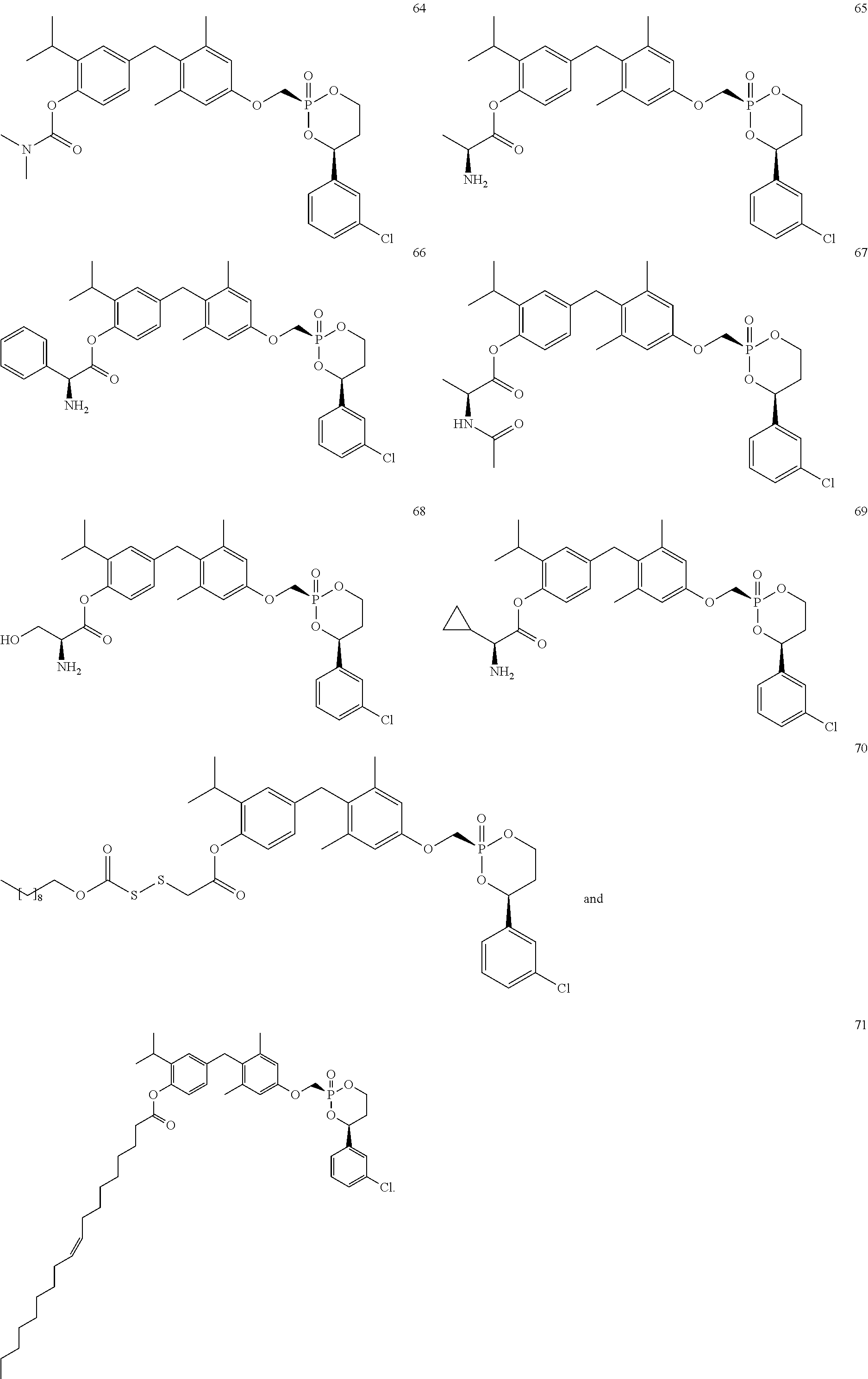

9. The compound of claim 1, stereoisomers thereof, pharmaceutically acceptable salts thereof, deuterium substitutes thereof, isomers thereof, prodrugs thereof, and metabolites thereof, wherein the formula I is formula III, 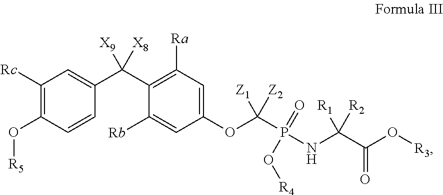
wherein Each of Ra and Rb is independently selected from the group consisting of CH₃, CD₃, Cl, Br, I and CF₃;
each R_{c} is selected from the group consisting of hydrogen, halogen, —CF₃, —OCF₃, cyano, optionally substituted —C₁-C₁₂ alkyl, optionally substituted —C₂-C₁₂ alkenyl, optionally substituted-C₂-C₁₂ alkynyl, optionally substituted —C₀₋₆alkyl-aryl, optionally substituted —C₀₋₆ alkyl-cycloalkyl, optionally substituted —C₀₋₆ alkyl-heterocycloalkyl, optionally substituted —C₃₋₈ cycloalkyl;
wherein R_{c} is optionally substituted with one to ten halogen, H or D;
wherein each of X₈, X₉, Z₁ and Z₂ is independently H, D or halo;
Z₃ is independently O, —CH₂—;
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl-C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —CONHR₆, —COOR₆, —CONR₆R₇, —CH₂OCOR₆, and —CH₂OCONHR₆;
wherein each of R₆ and R₇ is independently C₁₋₃₀ alkyl, C₁₋₃₀ alkenyl, or C₁₋₃₀ alkynyl, wherein the C₁₋₃₀ alkyl, the C₁₋₃₀ alkenyl, or the C₁₋₃₀ alkynyl is optionally substituted with one or more substituents each independently selected from the group consisting of halo, NH₂, NO₂, OH, CN, O—C₁₋₆ alkyl, C₆₋₁₀ aryl, and heteroaryl; or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl.

10. The compound of claim 9, stereoisomers thereof, pharmaceutically acceptable salts thereof, deuterium substitutes thereof, isomers thereof, prodrugs thereof, and metabolites thereof, wherein Z is O.

11. The compound of claim 9, stereoisomers thereof, pharmaceutically acceptable salts thereof, deuterium substitutes thereof, isomers thereof, prodrugs thereof, and metabolites thereof, wherein each of Z₁ and Z₂ is H.

12. The compound of claim 9, stereoisomers thereof, pharmaceutically acceptable salts thereof, deuterium substitutes thereof, isomers thereof, prodrugs thereof, and metabolites thereof, wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl.

13. The compound of claim 9, stereoisomers thereof, pharmaceutically acceptable salts thereof, deuterium substitutes thereof, isomers thereof, prodrugs thereof, and metabolites thereof, wherein R₃ is selected from the group consisting of C₁₋₃₀ alkyl, and C₅₋₁₀ cycloalkyl.

14. The compound of claim 9, stereoisomers thereof, pharmaceutically acceptable salts thereof, deuterium substitutes thereof, isomers thereof, prodrugs thereof, and metabolites thereof, wherein R_{c} is independently selected from the group consisting of —C₁-C₆ alkyl, —C₀₋₆ alkyl-aryl, —C₀₋₆ alkyl-cycloalkyl, —C₀₋₆ alkyl-heterocycloalkyl, and —C₃₋₈ cycloalkyl; wherein the R_{c} is optionally substituted with halo.

15. The compound of claim 1, stereoisomers thereof, pharmaceutically acceptable salts thereof, deuterium substitutes thereof, isomers thereof, prodrugs thereof, and metabolites thereof, wherein formula I is formula IV, 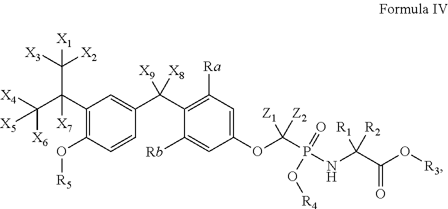
wherein each of R₁ and R₂ is independently selected from the group consisting of H, C₁₋₆ alkyl, C₁₋₆ haloalkyl, C₃₋₁₀ cycloalkyl, C₂₋₈ alkenyl, C₂₋₈ alkynyl, C₆₋₁₀ aryl, heteroaryl, C₁₋₆ alkyl C₆₋₁₀ aryl, and C₁₋₆ alkyl-heteroaryl, or R₁ and R₂ are combined with the atoms to which they are attached to form a C₃₋₁₀ cycloalkyl or a heterocyclyl;
wherein R₃ is selected from the group consisting of H, C₁₋₃₀ alkyl, C₅₋₁₀ cycloalkyl, C₁₋₃₀ haloalkyl, C₆₋₁₀ aryl, and C₆₋₁₀ aryl-C₁₋₈ alkyl, wherein R₃ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with one or more substituents each independently selected from the group consisting of halo, NH₂, NO₂, OH, CN, —C₁₋₆ alkyl, C₁₋₆ haloalkyl, and O—C₁₋₆ alkyl;
wherein R₅ is selected from the group consisting of H, —COR₆, —CONHR₆, —COOR₆, —CONR₆R₇, —CH₂OCOR₆, and —CH₂OCONHR₆;
wherein each of X₁, X₂, X₃, X₄, X₅, X₆, X₇, X₈ and X₉ is independently H, D or halo,
wherein each of Z₁ and Z₂ is independently H, D or halo;
wherein each of Ra and Rb is independently selected from the group consisting of CH₃, CD₃, Cl, Br, I or CF₃;
wherein each of R₆ and R₇ is independently C₁₋₃₀ alkyl, C₁₋₃₀ alkenyl, or C₁₋₃₀ alkynyl, wherein the C₁₋₃₀ alkyl, the C₁₋₃₀ alkenyl, or the C₁₋₃₀ alkynyl is optionally substituted with one or more substituents each independently selected from the group consisting of halo, NH₂, NO₂, OH, CN, O—C₁₋₆ alkyl, C₆₋₁₀ aryl, and heteroaryl; or R₆ and R₇ are combined with atoms to which they are attached to form a 5-10 membered heterocyclyl, and wherein the 5-10 membered heterocyclyl is optionally substituted with one or more substituents each independently selected from the group consisting of halo, NH₂, NO₂, OH, CN, —C₁₋₆ alkyl, C₁₋₆ haloalkyl, and O—C₁₋₆ alkyl.

16. The compound of claim 15, stereoisomers thereof, pharmaceutically acceptable salts thereof, deuterium substitutes thereof, isomers thereof, prodrugs thereof, and metabolites thereof, wherein each of Z₁ and Z₂ is H.

17. The compound of claim 15, stereoisomers thereof, pharmaceutically acceptable salts thereof, deuterium substitutes thereof, isomers thereof, prodrugs thereof, and metabolites thereof, wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl.

18. The compound of claim 15, stereoisomers thereof, pharmaceutically acceptable salts thereof, deuterium substitutes thereof, isomers thereof, prodrugs thereof, and metabolites thereof, wherein R₃ is selected from the group consisting of C₁₋₃₀ alkyl, and C₅₋₁₀ cycloalkyl.

19. The compound of claim 1, stereoisomers thereof, pharmaceutically acceptable salts thereof, deuterium substitutes thereof, isomers thereof, prodrugs thereof, and metabolites thereof, wherein formula I is Formula V, 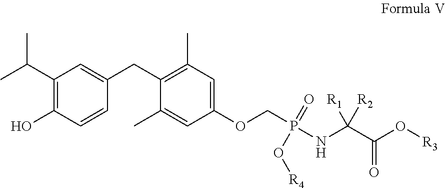
wherein R₁ and R₂ is independently selected from the group consisting of H, C₁₋₆ alkyl, C₆₋₁₀ aryl, heteroaryl, C₁₋₆ alkyl-C₆₋₁₀ aryl, and C₁₋₆ alkyl-heteroaryl;
wherein R₃ is selected from the group consisting of H, C₁₋₃₀ alkyl, and C₅₋₁₀ cycloalkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl.

20. The compound of claim 19, stereoisomers thereof, pharmaceutically acceptable salts thereof, deuterium substitutes thereof, isomers thereof, prodrugs thereof, and metabolites thereof, wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl.

21. The compound of claim 19, stereoisomers thereof, pharmaceutically acceptable salts thereof, deuterium substitutes thereof, isomers thereof, prodrugs thereof, and metabolites thereof, wherein R₃ is selected from the group consisting of C₁₋₃₀ alkyl, and C₅₋₁₀ cycloalkyl.

22. The compound of claim 1, stereoisomers thereof, pharmaceutically acceptable salts thereof, deuterium substitutes thereof, isomers thereof, prodrugs thereof, and metabolites thereof, wherein formula I is formula VI, 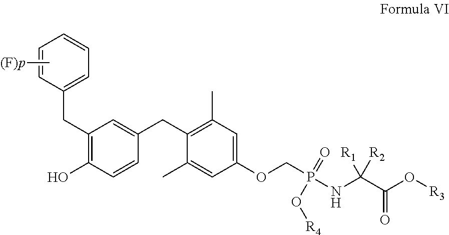
wherein R₁ and R₂ is independently selected from the group consisting of H, C₁₋₆ alkyl, C₆₋₁₀ aryl, heteroaryl, C₁₋₆ alkyl-C₆₋₁₀ aryl, and C₁₋₆ alkyl-heteroaryl; wherein the alkyl, aryl and heteroaryl is optionally substituted with halogen, C₁₋₆ alkyl and C₁₋₆ haloalkyl;
wherein R₃ is selected from the group consisting of H, C₁₋₃₀ alkyl, and C₅₋₁₀ cycloalkyl;
wherein R₄ is selected from the group consisting of C₆₋₁₀ aryl, C₁₋₆ alkyl-C₆₋₁₀ aryl, and 5-10 membered heteroaryl, wherein R₄ is optionally substituted with halo, —C₁₋₆ alkyl, C₁₋₆ haloalkyl, and O—C₁₋₆ alkyl;
p is an integer from 0-3.

23. The compound of claim 22, stereoisomers thereof, pharmaceutically acceptable salts thereof, deuterium substitutes thereof, isomers thereof, prodrugs thereof, and metabolites thereof, wherein the R₃ is C₁₋₆ alkyl, or C₅₋₁₀ cycloalkyl.

24. The compound of claim 22, stereoisomers thereof, pharmaceutically acceptable salts thereof, deuterium substitutes thereof, isomers thereof, prodrugs thereof, and metabolites thereof, wherein the R₃ is C₇-30 alkyl, or C₅₋₁₀ cycloalkyl.

25. The compound of claim 23, stereoisomers thereof, pharmaceutically acceptable salts thereof, deuterium substitutes thereof, isomers thereof, prodrugs thereof, and metabolites thereof, wherein the Ra is C₆₋₁₀ aryl; and C₆₋₁₀ the aryl is optionally substituted with halogen, C₁₋₆ alkyl.

26. The compound of claim 9, stereoisomers thereof, pharmaceutically acceptable salts thereof, deuterium substitutes thereof, isomers thereof, prodrugs thereof, and metabolites thereof, pharmaceutically acceptable salts thereof, deuterium substitutes thereof, isomers thereof, prodrugs thereof, and metabolites thereof, wherein the compound is selected from the group consisting of:

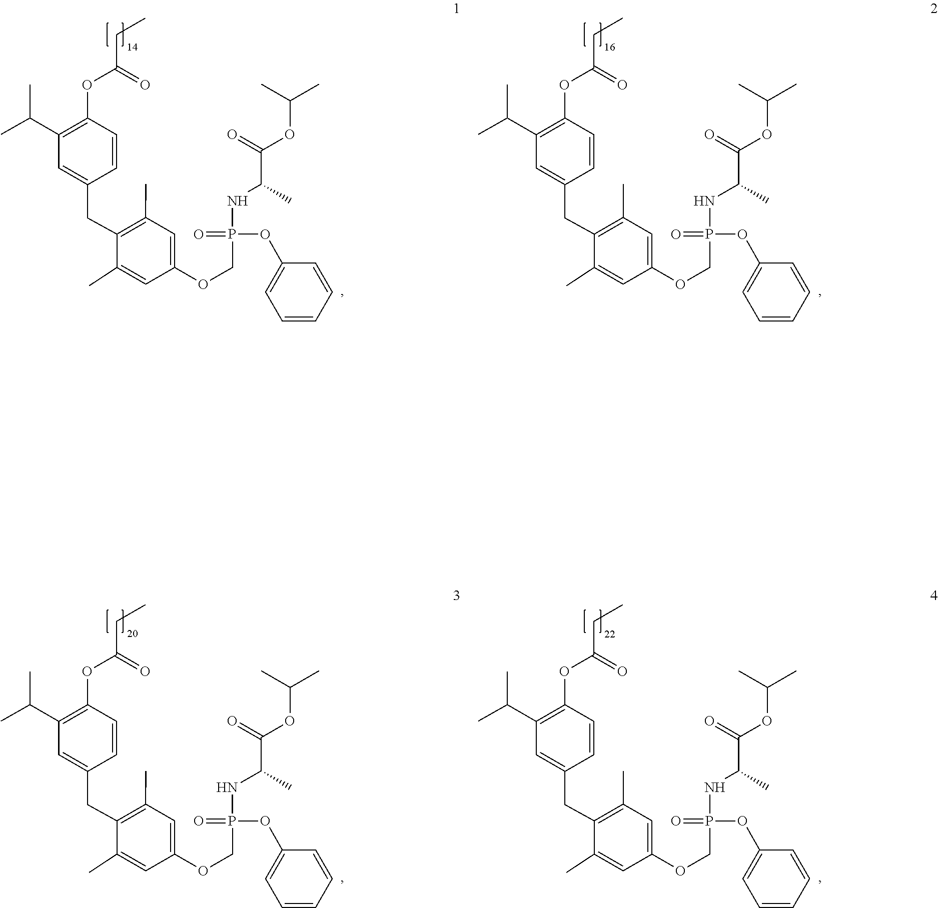

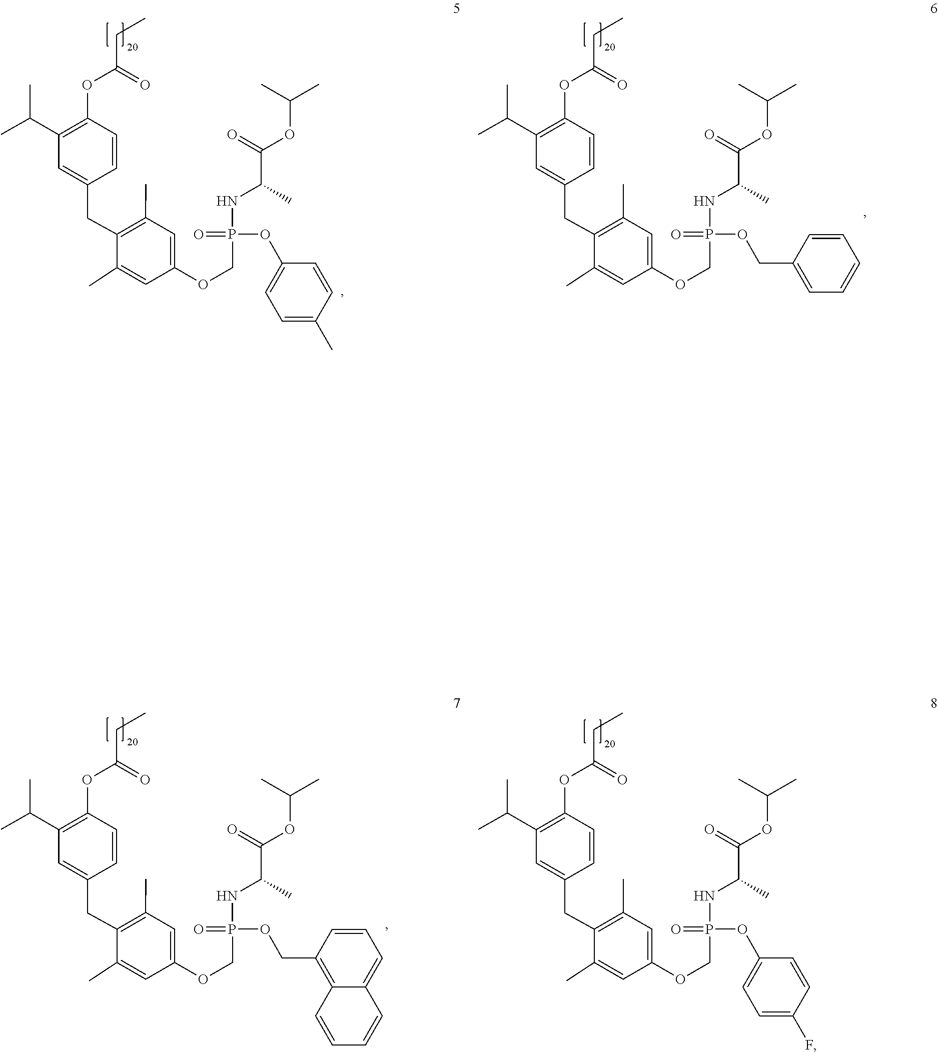

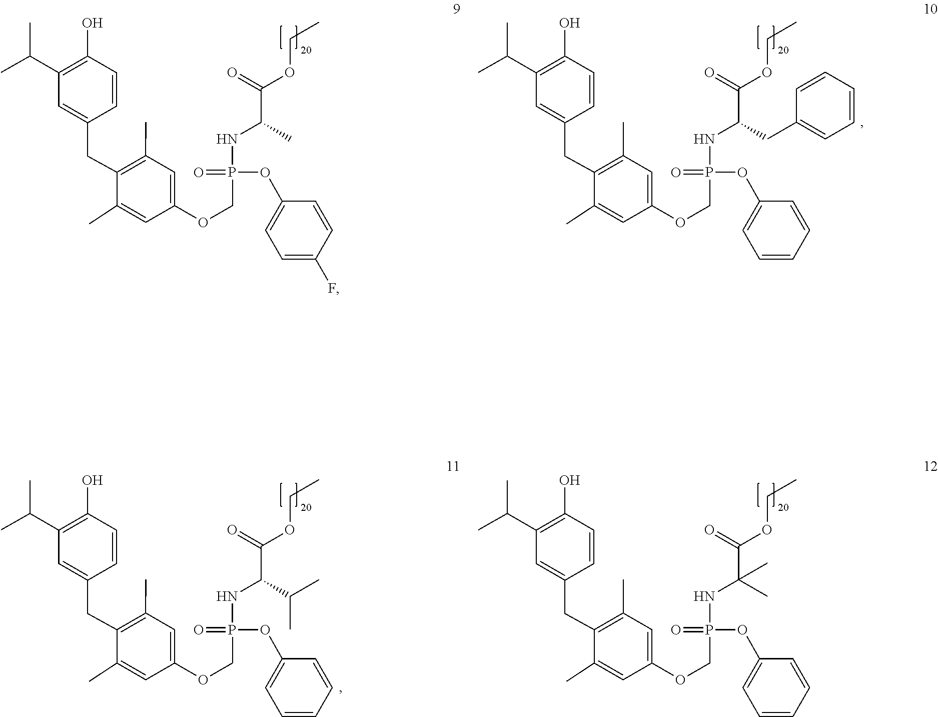

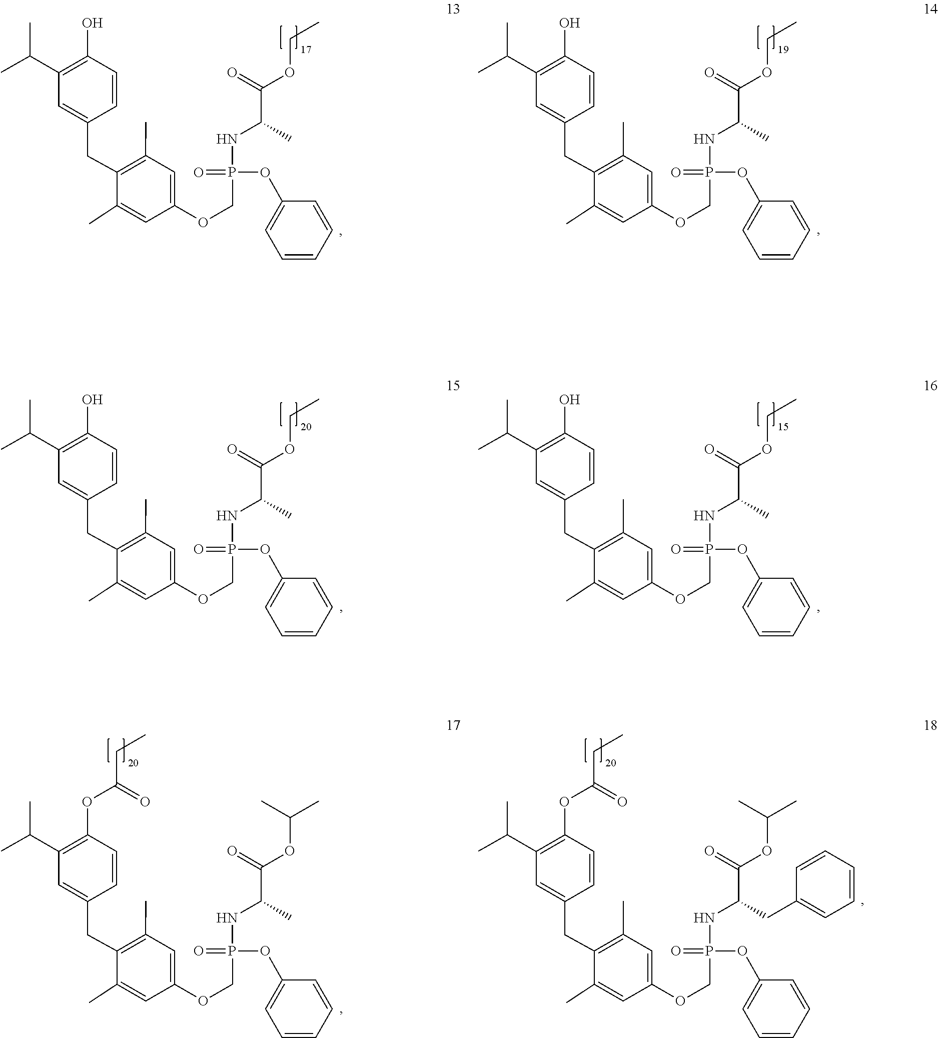

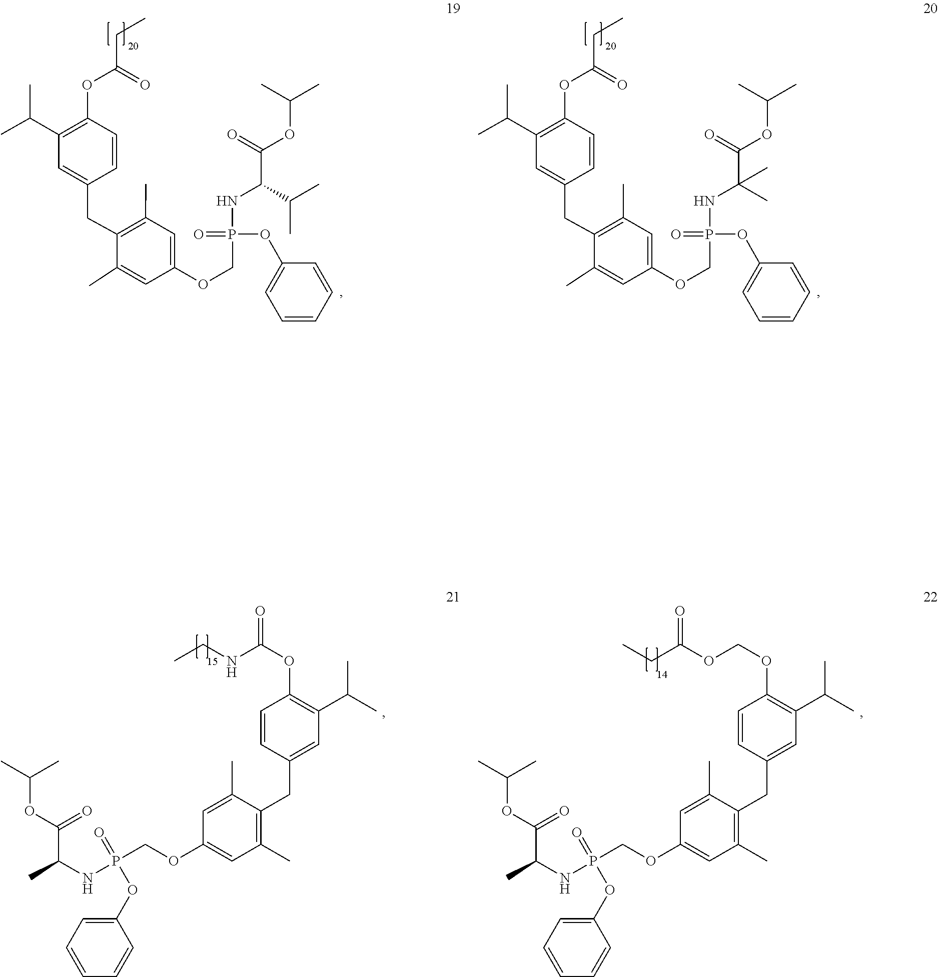

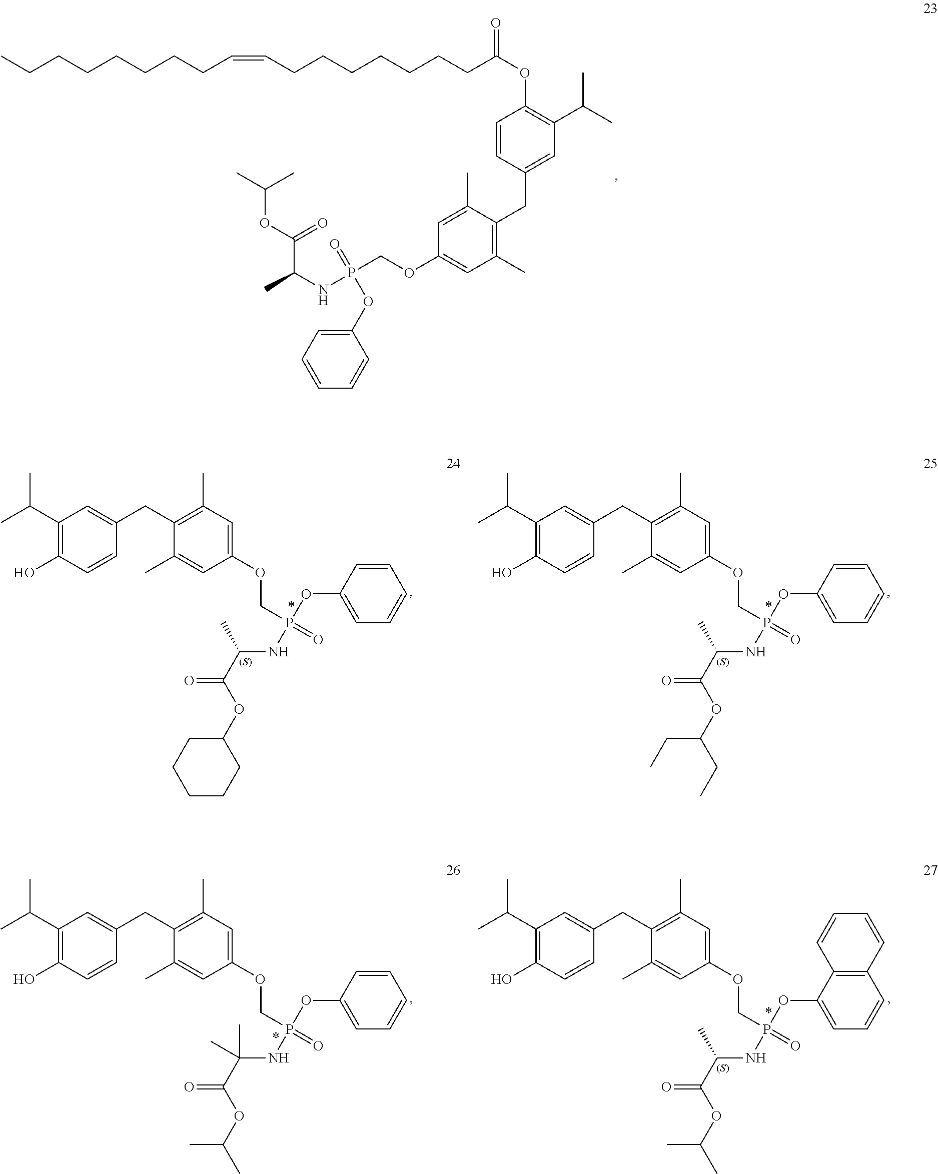

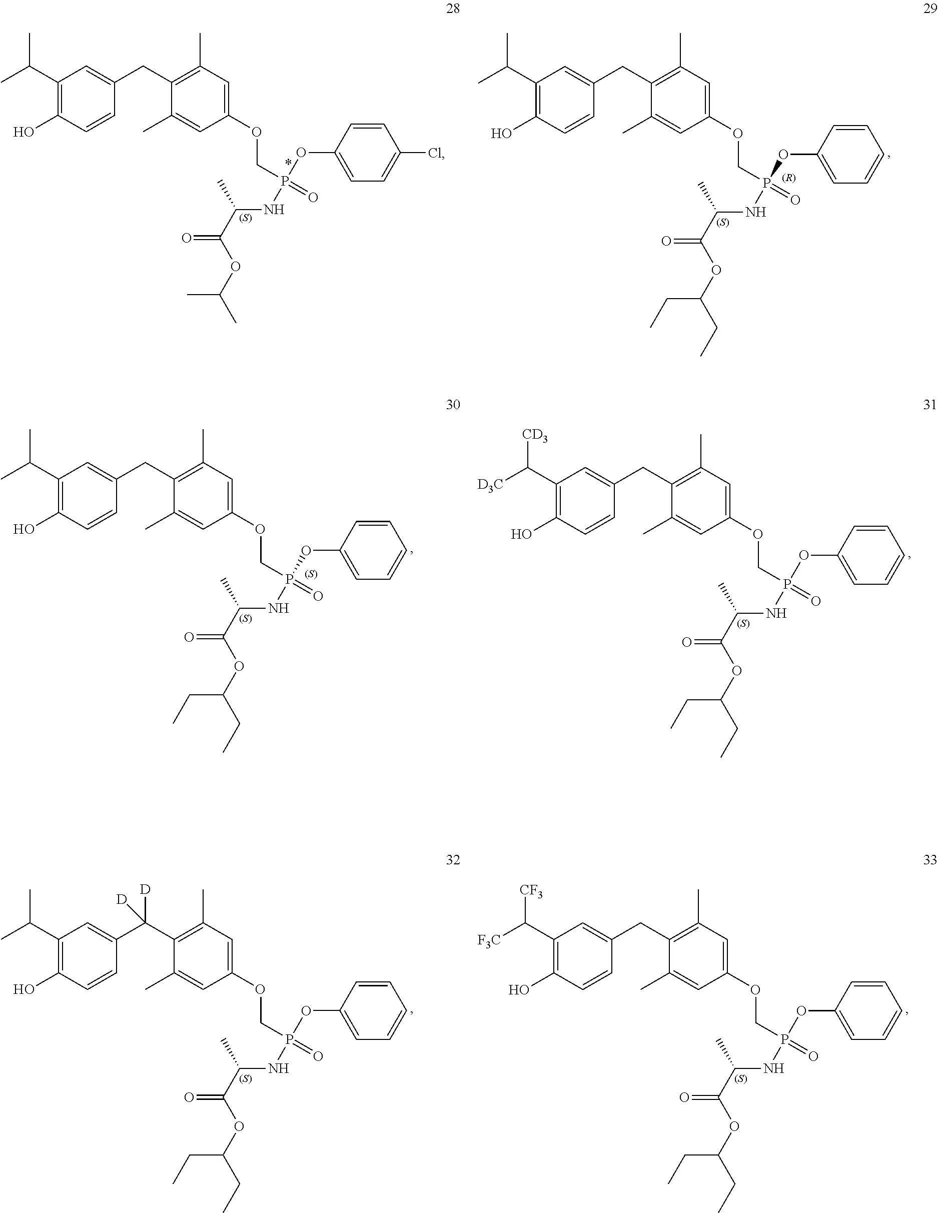

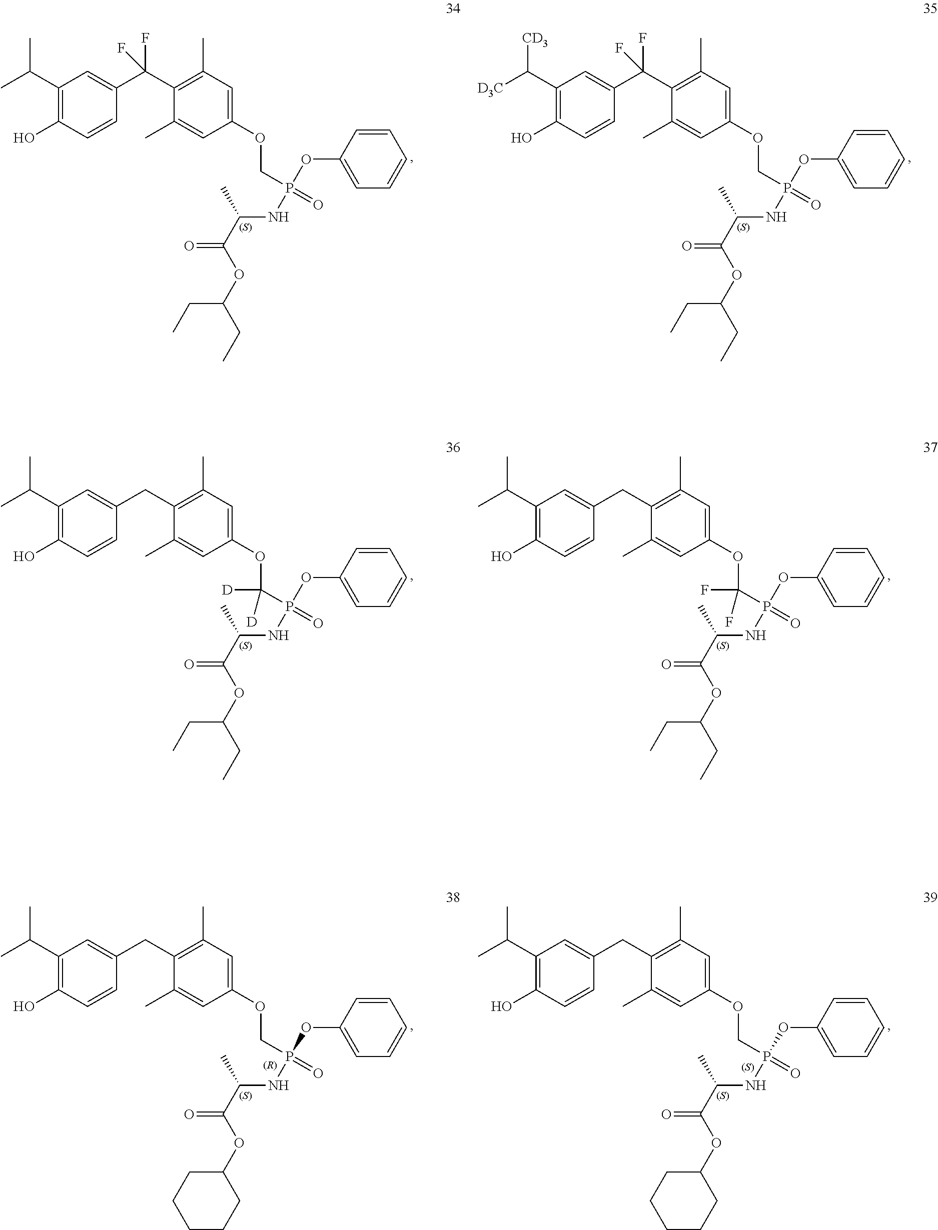

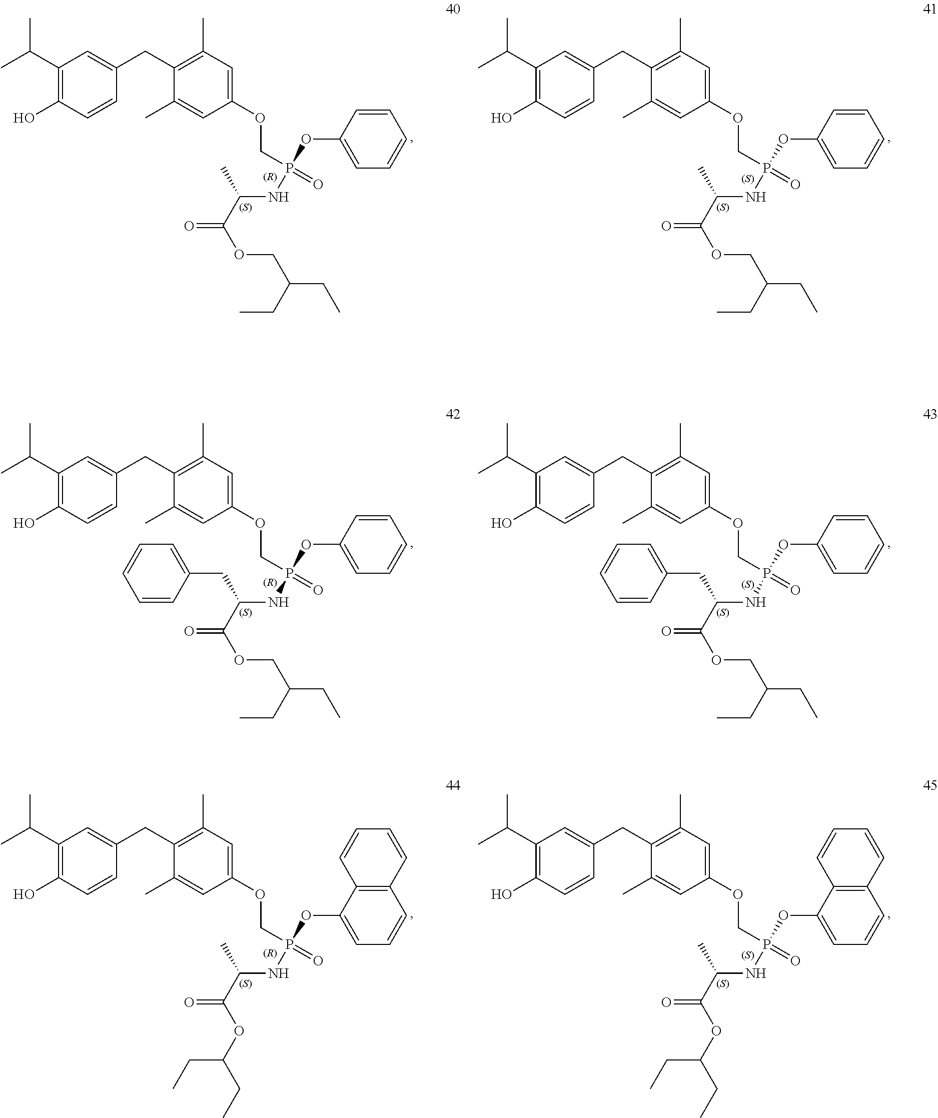

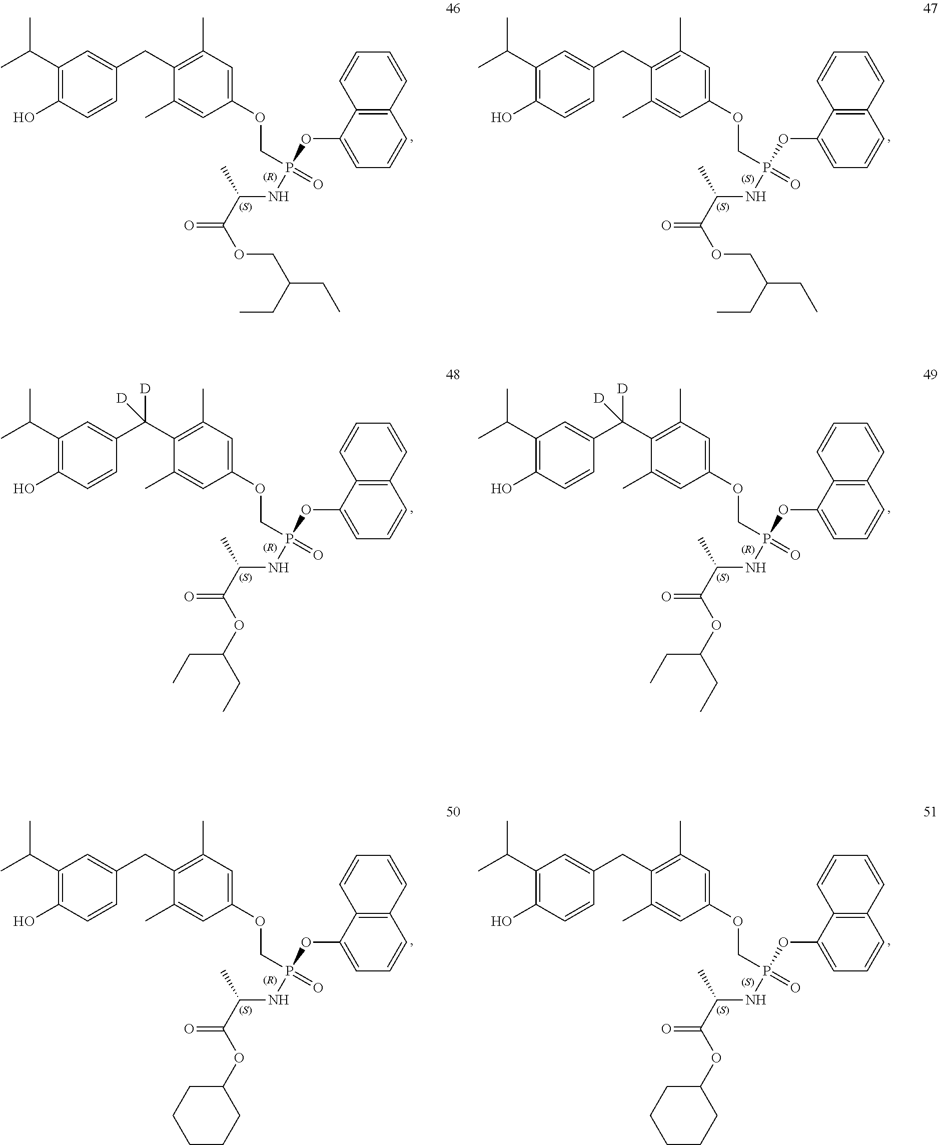

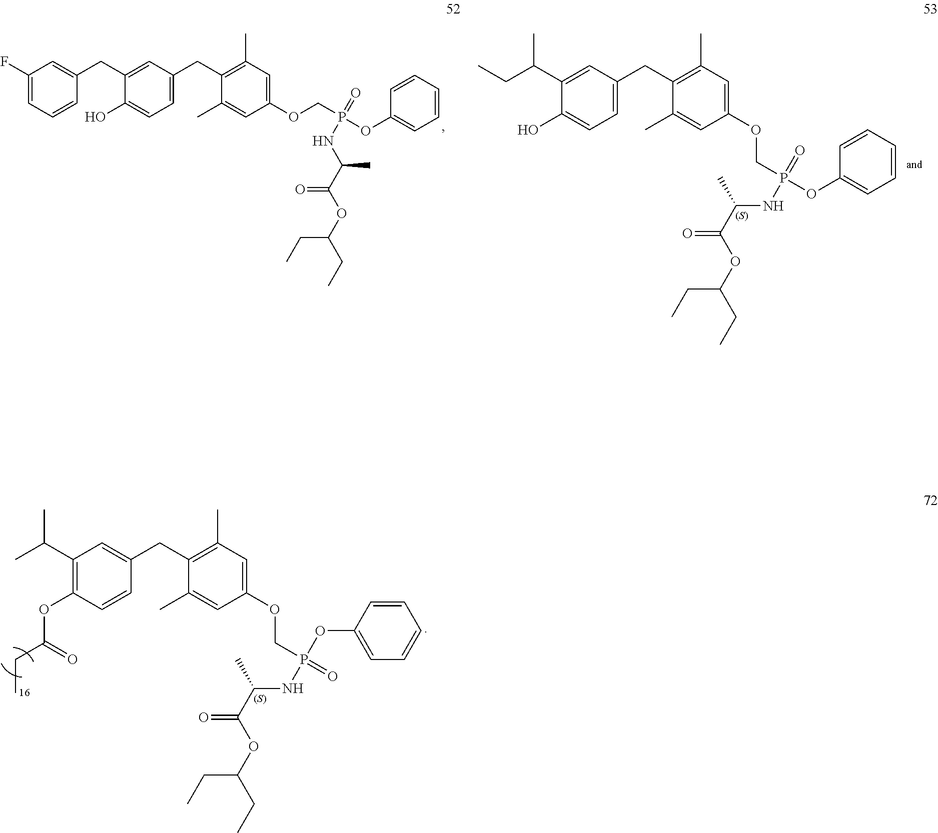

27. A pharmaceutical composition, comprising:
the compound of claim 1, a pharmaceutically acceptable salt thereof, a deuterium substitute thereof, a isomer thereof, a prodrug thereof, or a metabolite thereof; and
a pharmaceutically acceptable excipient.

28. A method for treating or preventing obesity, hyperlipidemia, hypercholesterolemia, diabetes mellitus, nonalcoholic steatohepatitis (NASH), hepatic steatosis, arteriosclerosis, cardiovascular disease, hypothyroidism, or thyroid cancer in a subject in need thereof, comprising the step of administering to the subject an effective amount of the compound of claim 1.
